(19) United States
(12) Patent Application Publication
(10) Pub. No.: US 2026/0250591 A1
(43) Pub. Date: Aug. 27, 2026

(54) FLUORINE-CONTAINING ETHER COMPOUND, LUBRICANT FOR MAGNETIC RECORDING MEDIUM, AND MAGNETIC RECORDING MEDIUM

(71) Applicant: RESONAC CORPORATION, Tokyo (JP)

(72) Inventors: Yutaka TANJI, Tokyo (JP); Kohei SAGA, Tokyo (JP)

(73) Assignee: RESONAC CORPORATION, Tokyo (JP)

(21) Appl. No.: 18/861,339

(22) PCT Filed: Apr. 16, 2024
(86) PCT No.: PCT/JP2024/015070

§ 371 Date: Oct. 29, 2024

(30) Foreign Application Priority Data
Apr. 28, 2023 (JP) 2023-074982

(51) Int. Cl. C10M 105/54; C08G 65/331; C10N 40/18; G11B 5/725

(52) U.S. Cl. CPC C10M 105/54; C08G 65/3318; G11B 5/7257; C10M 2213/0606; C10N 2040/18

(57) ABSTRACT

Provided is a fluorine-containing ether compound represented by the following formula. R¹—CH₂—R²[—CH₂—R³—CH₂—R²]ₓ—CH₂—R⁴ (x represents 1 or 2, R² represents a perfluoropolyether chain, R³ represents a divalent linking group having one to four polar groups, each R¹ and R⁴ represents an end group having one to four polar groups and having 1 to 50 carbon atoms, at least one of R¹ or R⁴ represents —O—X—CH(OH)—CH₂OH (X represents a divalent organic group having 2 to 30 carbon atoms, which may have at least one of one or two polar groups or one to three ether oxygen atoms, and X has at least one carbon atom which is not bonded to either the polar groups or the ether oxygen atoms.)

### TECHNICAL FIELD

The present invention relates to a fluorine-containing ether compound, a lubricant for a magnetic recording medium, and a magnetic recording medium.

Priority is claimed on Japanese Patent Application No. 2023-074982, filed Apr. 28, 2023, the content of which is incorporated herein by reference.

### BACKGROUND ART

In order to improve the recording density of a magnetic recording and reproducing device, development of a magnetic recording medium suitable for a high recording density has been promoted.

In the related art, there is a magnetic recording medium in which a recording layer is formed on a substrate and a protective layer made of carbon or the like is formed on the recording layer, as a magnetic recording medium. The protective layer protects information recorded on the recording layer and increases the slidability of a magnetic head. However, the durability of the magnetic recording medium is not sufficiently obtained only by providing the protective layer on the recording layer. Therefore, in general, a lubricating layer is formed by coating a surface of the protective layer with a lubricant.

As a lubricant used in a case of forming a lubricating layer of a magnetic recording medium, for example, a lubricant which contains a compound containing a polar group such as a hydroxy group or an amino group at an end of a fluorine-based polymer having a repeating structure containing —CF₂— has been proposed.

For example, Patent Documents 1 and 2 disclose a fluorine-containing ether compound which has a skeleton in which two perfluoropolyether chains are bonded to each other through a divalent linking group in which a methylene group (—CH₂—) is bonded to both ends of a glycerin structure (—O—CH₂—CH(OH)—CH₂—O—), and end groups containing a polar group are bonded to both ends of the skeleton through a methylene group.

Patent Documents 3 and 4 disclose a fluorine-containing ether compound which contains a methylene group and a group (—CH(OH)—) in which one hydrogen atom of the methylene group is substituted with a hydroxy group, has a skeleton in which two perfluoropolyether chains are bonded to each other through a divalent linking group containing two hydroxy groups, and has an end group which is an organic group containing a polar group bonded through a methylene group at both ends of the skeleton.

Patent Document 5 discloses a fluorine-containing ether compound in which three perfluoropolyether chains have a skeleton in which the perfluoropolyether chains are bonded to the methylene group through a glycerin structure, and end groups containing a polar group are bonded to both sides of the skeleton through a methylene group.

### CITATION LIST

### Patent Documents

Patent Document 1: U.S. patent Ser. No. 10/540,997
Patent Document 2: PCT International Publication No. WO2021/251335
Patent Document 3: Japanese Patent Publication No. 5743438
Patent Document 4: Japanese Patent Publication No. 6804981
Patent Document 5: PCT International Publication No. WO2022/215726

### SUMMARY OF INVENTION

### Technical Problem

In order to increase the capacity of a magnetic recording and reproducing device, development of a magnetic recording medium suitable for a high recording density has been promoted. In recent years, in order to improve the recording density of a magnetic recording medium, the distance between a magnetic head and a magnetic layer of the magnetic recording medium is required to be decreased to reduce a magnetic spacing (flying height). Therefore, the thickness of a lubricating layer in the magnetic recording medium is required to be further reduced.

However, in general, in a case where the thickness of the lubricating layer is reduced, the corrosion resistance of the magnetic recording medium tends to be degraded. Further, in a case where the floating amount of the magnetic head is reduced, a pickup in which a fluorine-containing ether compound in the lubricating layer adheres to the magnetic head may occur.

The present invention has been made in consideration of the above-described circumstances, and an object of the present invention is to provide a fluorine-containing ether compound which can form a lubricating layer having excellent corrosion resistance and capable of suppressing a pickup, and can be suitably used as a material of a lubricant for a magnetic recording medium.

Further, another object of the present invention is to provide a lubricant for a magnetic recording medium, which can form a lubricating layer containing the fluorine-containing ether compound of the present invention and having satisfactory corrosion resistance and a high pickup suppression effect.

Further, still another object of the present invention is to provide a magnetic recording medium which include a lubricating layer containing the fluorine-containing ether compound of the present invention and has satisfactory corrosion resistance and a high pickup suppression effect.

### Solution to Problem

The present invention includes the following aspects.

[1]A fluorine-containing ether compound represented by Formula (1).

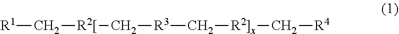

(In Formula (1), x represents an integer of 1 to 2. R² represents a perfluoropolyether chain. (x+1) pieces of R²'s may be partially or entirely the same as or different from each other. R³ represents a divalent linking group having one to four polar groups. In a case where x represents 2, two R³'s may be the same as or different from each other. Each R¹ and R⁴ represents an end group having one to four polar groups and 1 to 50 carbon atoms. R¹ and R⁴ may be the same as or different from each other. At least one of R or R⁴ represents an end group represented by Formula (2).

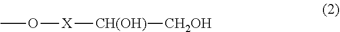

(In Formula (2), X represents a divalent organic group having 2 to 30 carbon atoms, which may have at least one of one or two polar groups or one to three ether oxygen atoms. X has at least one carbon atom which is not bonded to either the polar groups or the ether oxygen atoms.)

[2] The fluorine-containing ether compound according to [1], in which the end group represented by Formula (2) is a group represented by any of Formulae (2-1) to (2-7).

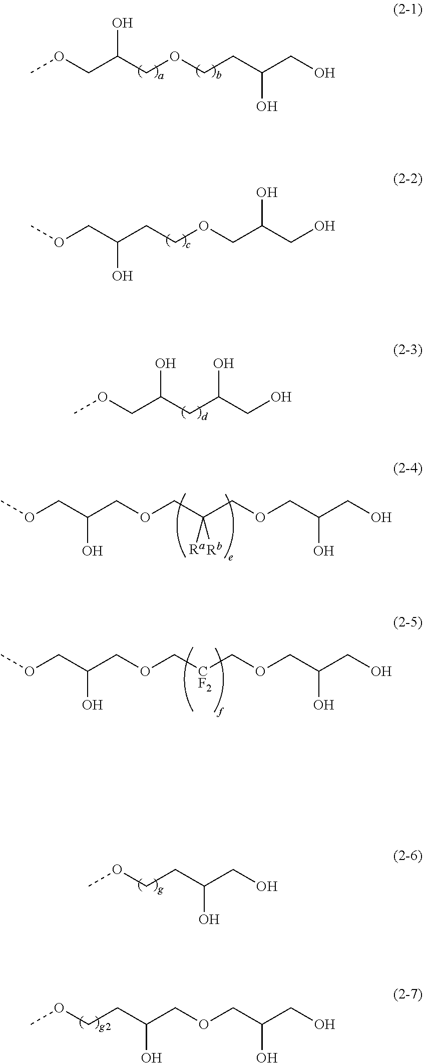

(In Formula (2-1), a represents an integer of 1 to 8. b represents an integer of 1 to 7.)

(In Formula (2-2), c represents an integer of 1 to 7.)

(In Formula (2-3), d represents an integer of 1 to 6.)

(In Formula (2-4), e represents an integer of 1 to 6. e pieces of R^{a}'s and R^{b}'s each independently represent a hydrogen atom or a methyl group.)

(In Formula (2-5), f represents an integer of 1 to 6.)

(In Formula (2-6), g represents an integer of 1 to 6.)

(In Formula (2-7), g2 represents an integer of 1 to 6.)

[3] The fluorine-containing ether compound according to [1] or [2], in which R¹ and R⁴ in Formula (1) each independently represent an end group represented by Formula (2).

[4] The fluorine-containing ether compound according to any one of [1] to [3], in which R¹ and R⁴ in Formula (1) are the same as each other.

[5] The fluorine-containing ether compound according to [1] or [2], in which one of R¹ or R⁴ in Formula (1) represents an end group represented by Formula (2) and the other represents an end group represented by Formula (3) and not corresponding to Formula (2).

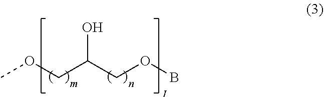

(In Formula (3), l represents an integer of 1 to 3. l pieces of m's each independently represent an integer of 1 to 6. l pieces of n's each independently represent an integer of 1 to 6. In one repeating unit, at least one of m or n represents 1. B represents an alkyl group which may have only one polar group, an organic group having one or more carbon-carbon unsaturated bonds, or a hydrogen atom.)

[6] The fluorine-containing ether compound according to any one of [1], [2], or [5], in which one of R¹ or R⁴ in Formula (1) represents an end group represented by Formula (2) and the other represents an end group represented by Formula (3-1) or (3-2).

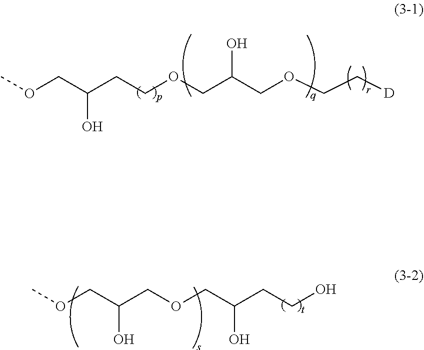

(In Formula (3-1), p represents an integer of 0 to 3. q represents an integer of 0 to 2. r represents an integer of 0 to 5. The total value of p and r is in a range of 1 to 5. D represents a polar group, a vinyl group, an ethynyl group, or an aryl group which may have a substituent.)

(In Formula (3-2), s represents an integer of 0 to 2. t represents an integer of 1 to 5.)

[7] The fluorine-containing ether compound according to any one of [1] to [6], in which x pieces of R³'s in Formula (1) each independently represent a divalent linking group having 3 to 50 carbon atoms, which has 1 to 3 hydroxy groups and has an oxygen atom at both ends bonded to adjacent methylene groups.

[8] The fluorine-containing ether compound according to any one of [1] to [7], in which x pieces of R³'s in Formula (1) each independently represent any one selected from the group consisting of linking groups represented by Formulae (4-1) to (4-6).

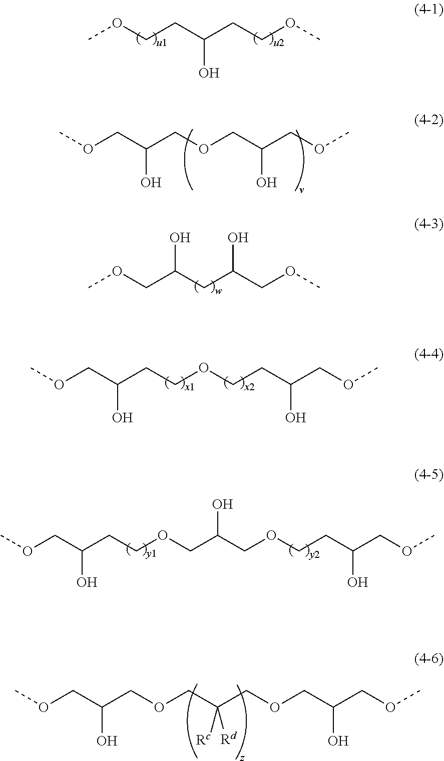

(In Formula (4-1), u1 represents an integer of 0 to 6, and u2 represents an integer of 0 to 6. Here, at least one of u1 or u2 represents 0. The oxygen atom at the left end of Formula (4-1) is bonded to the methylene group on the R¹ side in Formula (1), and the oxygen atom at the right end is bonded to the methylene group on the R⁴ side in Formula (1).)

(In Formula (4-2), v represents an integer of 1 or 2. The oxygen atom at the left end of Formula (4-2) is bonded to the methylene group on the R¹ side in Formula (1), and the oxygen atom at the right end is bonded to the methylene group on the R⁴ side in Formula (1).)

(In Formula (4-3), w represents an integer of 0 to 6. The oxygen atom at the left end of Formula (4-3) is bonded to the methylene group on the R¹ side in Formula (1), and the oxygen atom at the right end is bonded to the methylene group on the R⁴ side in Formula (1).)

(In Formula (4-4), x1 represents an integer of 0 to 5, and x2 represents an integer of 0 to 5. Here, at least one of x1 or x2 represents an integer of 1 to 5. The oxygen atom at the left end of Formula (4-4) is bonded to the methylene group on the R¹ side in Formula (1), and the oxygen atom at the right end is bonded to the methylene group on the R⁴ side in Formula (1).

(In Formula (4-5), y1 represents an integer of 1 to 5, and y2 represents an integer of 1 to 5. The oxygen atom at the left end of Formula (4-5) is bonded to the methylene group on the R¹ side in Formula (1), and the oxygen atom at the right end is bonded to the methylene group on the R⁴ side in Formula (1).)

(In Formula (4-6), z represents an integer of 1 to 6. z pieces of R^{c}'s and R^{d}'s each independently represent a hydrogen atom, a fluorine atom, or a methyl group. The oxygen atom at the left end of Formula (4-6) is bonded to the methylene group on the R¹ side in Formula (1), and the oxygen atom at the right end is bonded to the methylene group on the R⁴ side in Formula (1).)

[9] The fluorine-containing ether compound according to any one of [1] to [8], in which a total number of the polar groups of R¹, the polar groups of R⁴, and the polar groups of x pieces of R³'s in Formula (1) is in a range of 6 to 12.

[10] The fluorine-containing ether compound according to any one of [1] to [9], in which (x+1) pieces of R²'s in Formula (1) each independently represent a perfluoropolyether chain represented by Formula (5).

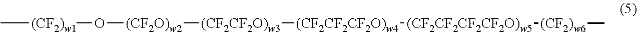

(In Formula (5), each w2, w3, w4, and w5 represents an average degree of polymerization, and each independently represent 0 to 20. Here, all of w2, w3, w4, and w5 do not represent 0 at the same time. Each w1 and w6 represents an average value representing the number of CF₂'s, and each independently represent 1 to 3. The arrangement order of (CF₂O), (CF₂CF₂O), (CF₂CF₂CF₂O), and (CF₂CF₂CF₂CF₂O), which are the repeating units in Formula (5), is not particularly limited.)

[11] The fluorine-containing ether compound according to any one of [1] to [10], in which (x+1) pieces of R² in Formula (1) each independently represent any one selected from the group consisting of perfluoropolyether chains represented by Formulae (5-1) to (5-4).

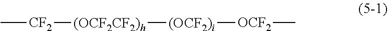

(In Formula (5-1), each h and i represents an average degree of polymerization, where h represents 1 to 20 and i represents 0 to 20.)

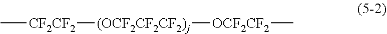

(In Formula (5-2), j represents an average degree of polymerization, and represents 1 to 15.)

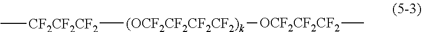

(In Formula (5-3), k represents an average degree of polymerization, and represents 1 to 10.)

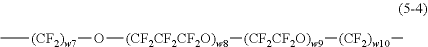

(In Formula (5-4), each w8 and w9 represents an average degree of polymerization, and each independently represents 1 to 20. Each w7 and w10 represents an average value representing the number of CF₂'s, and each independently represent 1 to 2.)

[12] The fluorine-containing ether compound according to any one of [1] to [11], in which the fluorine-containing ether compound has a number-average molecular weight of 500 to 10000.

[13]A lubricant for a magnetic recording medium, including: the fluorine-containing ether compound according to any one of [1] to [12].

[14]A magnetic recording medium, which is provided with at least a magnetic layer, a protective layer, and a lubricating layer in this order on a substrate, in which the lubricating layer contains the fluorine-containing ether compound according to any one of [1] to [12].

[15] The magnetic recording medium according to [14], in which the lubricating layer has an average film thickness of 0.5 nm to 2.0 nm.

### Advantageous Effects of Invention

The fluorine-containing ether compound of the present invention is a compound represented by Formula (1), and is suitable as a material of a lubricant for a magnetic recording medium.

Since the lubricant for a magnetic recording medium of the present invention contains the fluorine-containing ether compound of the present invention, it is possible to form a lubricating layer having excellent corrosion resistance and a high pickup suppression effect.

The magnetic recording medium of the present invention includes a lubricating layer containing the fluorine-containing ether compound of the present invention. Therefore, the magnetic recording medium of the present invention has satisfactory corrosion resistance, a high pickup suppression effect, and excellent reliability and durability. In addition, since the magnetic recording medium of the present invention includes a lubricating layer having satisfactory corrosion resistance and a suppressed pickup, the thickness of the lubricating layer can be reduced, and the floating amount of the magnetic head can be further reduced.

### BRIEF DESCRIPTION OF DRAWINGS

FIG. 1 A schematic cross-sectional view showing an embodiment of a magnetic recording medium according to the present invention.

### DESCRIPTION OF EMBODIMENTS

In order to solve the above-described problems, the present inventors have conducted intensive examination as described below.

In the related art, as a material of a lubricant for a magnetic recording medium (hereinafter, also referred to as “lubricant”) which is applied to a surface of a protective layer, a fluorine-containing ether compound containing a polar group such as a hydroxy group has been preferably used.

Some of the fluorine-containing ether compounds containing a polar group are formed such that an end group having a plurality of polar groups is disposed at the end of the chain structure. In addition, examples of the fluorine-containing ether compound include a compound having a plurality of perfluoropolyether chains, in which a linking group having a polar group is disposed between adjacent perfluoropolyether chains.

However, in a case where a lubricating layer having a small thickness is formed on a protective layer using the fluorine-containing ether compound having a polar group in the related art, it is difficult to realize a lubricating layer having satisfactory corrosion resistance and a high pickup suppression effect as described below.

That is, the polar group in the fluorine-containing ether compound is bonded to the active point on the protective layer to improve the adhesion of the lubricating layer to the protective layer. In a case where the adhesion of the lubricating layer to the protective layer is insufficient, a state of coating the protective layer with the lubricating layer is insufficient, water, which causes corrosion, is likely to be taken into the protective layer, and thus sufficient corrosion resistance cannot be obtained. Further, in a case where the adhesion of the lubricating layer to the protective layer is insufficient, the lubricating layer which has floated from the protective layer may come into collision with the magnetic head, which may cause a pickup.

In addition, the polar group in the fluorine-containing ether compound is involved not only in the interaction with the protective layer by being bonded to the active point on the protective layer, but also in the intramolecular or intermolecular interaction. In a case where the polar group involved in the intermolecular interaction between the fluorine-containing ether compounds is insufficient, the fluorine-containing ether compound in the lubricating layer is easily moved from the protective layer to the magnetic head, which is likely to cause a pickup.

Therefore, the fluorine-containing ether compound is required to have a sufficient amount of the polar group involved in the interaction with the protective layer and the polar group involved in the intermolecular interaction so that a lubricating layer having satisfactory corrosion resistance and a suppressed pickup can be formed. However, in a case where the number of polar groups in the fluorine-containing ether compound is increased, the hydrophobicity of the lubricating layer containing the fluorine-containing ether compound is lowered, and thus sufficient corrosion resistance cannot be obtained.

Therefore, the present inventors have repeatedly conducted intensive examination by focusing on the strength of polarity of the polar group contained in the fluorine-containing ether compound and the interaction of the polar groups. As a result, it was considered that at least some of the polar groups in the fluorine-containing ether compound may be hydroxy groups having a 1,2-diol structure, which are disposed at the end of the perfluoropolyether chain. In this case, as described below, a hydroxy group capable of interacting with the protective layer and a hydroxy group capable of being involved in the intermolecular interaction can be ensured while a decrease in hydrophobicity due to the hydroxy group contained in the 1,2-diol structure is suppressed.

In the 1,2-diol structure (—CH(OH)—CH₂OH), since carbon atoms, to which hydroxy groups are bonded, are bonded to each other, the distance between the hydroxy groups is short, and steric repulsion and electrostatic repulsion between the hydroxy groups are likely to occur. Therefore, two hydroxy groups contained in the 1,2-diol structure are disposed in opposite directions with respect to the carbon atom to which the 1,2-diol structure is bonded. Therefore, the dipole moments generated by two hydroxy groups contained in the 1,2-diol structure cancel each other, and an increase in polarity of the entire fluorine-containing ether compound molecules is suppressed.

In addition, the distance between the active points on the protective layer is sufficiently large as compared with the distance between the hydroxy groups contained in the 1,2-diol structure. Moreover, two hydroxy groups contained in the 1,2-diol structure are disposed in opposite directions with respect to the carbon atom to which the 1,2-diol structure is bonded. Therefore, two hydroxy groups contained in the 1,2-diol structure are not directed to be close to the protective layer at the same time, and only one hydroxy group between the two hydroxy groups can interact with the active point on the protective layer. Therefore, the other hydroxy group between the two hydroxy groups can be involved in the intermolecular interaction between the fluorine-containing ether compounds.

In the related art, the structure of the fluorine-containing ether compound used as a lubricant has been examined such that as many polar groups as possible in the compound are disposed to easily interacted with the active point on the protective layer in order to improve adhesion to the protective layer. Therefore, a structure in which adjacent hydroxy groups are directed in opposite directions on the protective layer tends to be avoided because the carbon atoms to which the hydroxy groups are bonded are bonded to each other.

However, the present inventors have ensured a polar group which can be involved in the intermolecular interaction between the fluorine-containing ether compounds by intentionally using some polar groups in the fluorine-containing ether compound as the hydroxy groups of the 1,2-diol structure. In addition, the present inventors have considered that the number of polar groups which can interact with the active point on the protective layer may be adjusted as necessary.

Further, the present inventors have repeatedly conducted examination on a fluorine-containing ether compound in which a 1,2-diol structure is disposed at one or both ends of a perfluoropolyether chain, in order to improve the corrosion resistance of a lubricating layer containing the compound and to suppress the pickup.

As a result, it has been found that a fluorine-containing ether compound having a skeleton having two or three perfluoropolyether chains, in which a divalent linking group having one to four polar groups is disposed between adjacent perfluoropolyether chains, end groups having 1 to 50 carbon atoms and one to four polar groups are disposed at both ends of the skeleton, and at least one of the two end groups is an end group represented by Formula (2), which has at least one carbon atom that is not bonded to either the polar groups or ether oxygen atoms and has a 1,2-diol structure, may be obtained, thereby completing the present invention.

In such a fluorine-containing ether compound, since the number of polar groups is appropriate and some of the polar groups are hydroxy groups having a 1,2-diol structure which are disposed at the end of the perfluoropolyether chain, polar groups capable of interacting with the protective layer and polar groups capable of being involved in the intermolecular interaction can be sufficiently ensured while a decrease in hydrophobicity due to the polar groups is suppressed.

Further, the end group represented by Formula (2), which is disposed at least at one end, has at least one carbon atom that is not bonded to either the polar groups or the ether oxygen atoms. Since this carbon atom has an extremely low affinity for water, the interaction between the fluorine-containing ether compound molecule and water is strongly hindered. Therefore, even in a case where the fluorine-containing ether compound contains a plurality of polar groups, the hydrophobicity is sufficiently obtained. In addition, since the movement of the above-described carbon atom is suppressed, the carbon atom imparts moderate rigidity to the fluorine-containing ether compound molecule, and suppresses the intramolecular interaction between the polar groups, and thus the polar groups are likely to be involved in the intermolecular interaction between the fluorine-containing ether compounds.

In addition, in the fluorine-containing ether compound, a divalent linking group having one to four polar groups is bonded between two or three perfluoropolyether chains. In the divalent linking group, the distance between adjacent end groups sandwiching the perfluoropolyether chain and the distance between the other adjacent divalent linking groups sandwiching the perfluoropolyether chain in a case of having three perfluoropolyether chains are also determined to be appropriate by the perfluoropolyether chains. Therefore, the interaction between the polar group of the divalent linking group and the protective layer is unlikely to be inhibited by the polar group of the adjacent end group or the other adjacent divalent linking group, and can be involved in the interaction with the protective layer.

As described above, the fluorine-containing ether compound can maintain low polarity while ensuring the number of polar groups involved in the interaction with the protective layer, and has sufficient hydrophobicity. Therefore, the lubricant containing this compound is unlikely to take in water, which causes corrosion, and thus the corrosion resistance is excellent. In addition, the above-described fluorine-containing ether compound easily generates a polar group which is not involved in the interaction with the protective layer and the intramolecular interaction, and the polar group easily forms an intermolecular interaction. Therefore, the fluorine-containing ether compound in the lubricating layer is unlikely to move from the protective layer to the magnetic head, and thus the pickup can be suppressed.

On the contrary, for example, in a case where the end group disposed at at least one end has no carbon atom that is not bonded to any of the polar group or the ether oxygen atom, and the fluorine-containing ether compound is extremely flexible, the polar group of the fluorine-containing ether compound easily form an intramolecular interaction, and thus the polar group is unlikely to be involved in an intermolecular interaction.

In addition, for example, in a case where all of a plurality of polar groups in the fluorine-containing ether compound are disposed with a sufficient distance between adjacent polar groups, all the polar groups are likely to be involved in the interaction with the protective layer, and accordingly, the interaction between the fluorine-containing ether compounds due to the polar groups is not sufficiently obtained.

In addition, for example, in a case where the fluorine-containing ether compound has only one perfluoropolyether chain and does not have a structure in which a divalent linking group having a polar group is disposed between adjacent perfluoropolyether chains, a central portion of the chain structure of the fluorine-containing ether compound cannot be sufficiently adsorbed on the protective layer. Therefore, since the state of coating the protective layer with the lubricating layer is insufficient, water, which causes corrosion, is likely to be taken into the protective layer, and thus the corrosion resistance is not sufficiently obtained. Further, the lubricating layer is likely to be in a state of floating from the protective layer, and thus a pickup is likely to occur due to the collision between the lubricating layer floating from the protective layer and the magnetic head.

Further, the present inventors have confirmed that a lubricating layer having satisfactory corrosion resistance and a high pickup suppression effect can be formed even in a case where the thickness thereof is reduced by using a lubricant containing the fluorine-containing ether compound described above, thereby completing the present invention.

Hereinafter, the fluorine-containing ether compound, the lubricant for a magnetic recording medium, and the magnetic recording medium of the present invention will be described in detail. Further, the present invention is not limited to the embodiments described below.

### [Fluorine-Containing Ether Compound]

The fluorine-containing ether compound of the present embodiment is represented by Formula (1).

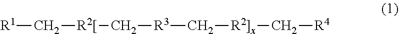

(In Formula (1), x represents an integer of 1 to 2. R² represents a perfluoropolyether chain. (x+1) pieces of R²'s may be partially or entirely the same as or different from each other. R³ represents a divalent linking group having one to four polar groups. In a case where x represents 2, two R³'s may be the same as or different from each other. R¹ and R⁴ represent an end group having one to four polar groups and 1 to 50 carbon atoms. R¹ and R⁴ may be the same as or different from each other. At least one of R¹ or R⁴ represents an end group represented by Formula (2).

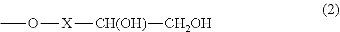

(In Formula (2), X represents a divalent organic group having 2 to 30 carbon atoms, which may have at least one of one or two polar groups or one to three ether oxygen atoms. X has at least one carbon atom which is not bonded to either the polar groups or the ether oxygen atoms.)

As shown in Formula (1), the fluorine-containing ether compound of the present embodiment has a skeleton in which a divalent linking group represented by R³ and two or three perfluoropolyether chains (hereinafter, also referred to as a PFPE chain) represented by R² are linked through a methylene group. An end group represented by R¹ is bonded to one end of the skeleton through a methylene group, and an end group represented by R⁴ is bonded to the other end of the skeleton through a methylene group.

In the fluorine-containing ether compound represented by Formula (1), x represents 1 or 2, and the number (x+1) of PFPE chains represented by R² is 2 or 3. Therefore, in the fluorine-containing ether compound represented by Formula (1), R³ which is a divalent linking group having one to four polar groups is disposed between adjacent R²'s, which is different from a case of a compound in which the number of PFPE chains is 1. In this manner, the fluorine-containing ether compound represented by Formula (1) can form a lubricating layer which has excellent adhesion to the protective layer and in which corrosion and the pickup of the magnetic recording medium are unlikely to occur, for example, as compared with a compound in which the number of PFPE chains is 1. In addition, in the fluorine-containing ether compound represented by Formula (1), the molecular size is suppressed from being increased as compared with a compound in which the number of PFPE chains is 4 or more, and the compound can move freely. Therefore, the fluorine-containing ether compound represented by Formula (1) can form a lubricating layer having a thin and uniform film thickness, which is likely to wet and spread on the protective layer, as compared with a compound in which the number of PFPE chains is 4 or more.

In the fluorine-containing ether compound represented by Formula (1), R¹, R³, and R⁴ each have one to four polar groups. The total number of the polar groups in R¹, the polar groups in R⁴, and the polar groups in x pieces of R³'s is preferably in a range of 6 to 12, more preferably in a range of 7 to 10, and most preferably 7 or 8.

In a case where the total number of the above-described polar groups is 6 or more, the interaction between the polar group of the fluorine-containing ether compound and the protective layer is effectively obtained. As a result, a fluorine-containing ether compound which can form a lubricating layer having satisfactory adhesion to the protective layer is obtained. Therefore, a lubricating layer having more excellent pickup resistance is obtained. Further, in a case where the total number of the above-described polar groups is 12 or less, it is possible to prevent water, which causes corrosion, from taking in because the polarity of the fluorine-containing ether compound is extremely high. Therefore, a lubricating layer having more excellent corrosion resistance can be formed.

(End Group Represented by R¹ and R⁴)

R¹ and R⁴ represent an end group having one to four polar groups and 1 to 50 carbon atoms. R¹ and R⁴ may be the same as or different from each other. At least one of R¹ or R⁴ represents an end group represented by Formula (2).

In a case where one of R¹ or R⁴ represents an end group that does not correspond to Formula (2), the number of polar groups contained in the end group that does not correspond to Formula (2) in R¹ and R⁴ is 1 or more. Further, the end group represented by Formula (2) contains at least two hydroxy groups of a 1,2-diol structure, and has two or more polar groups. Therefore, in a case where a lubricating layer is formed on the protective layer by using the lubricant containing the fluorine-containing ether compound represented by Formula (1), a polar group which can interact with the protective layer and a polar group which can be involved in the intermolecular interaction are obtained by the polar groups included in R¹ and R⁴, even in a case where one of R¹ and R⁴ represents an end group represented by Formula (2) or in a case where both of R¹ and R⁴ represent an end group represented by Formula (2). As a result, the lubricating layer containing the fluorine-containing ether compound represented by Formula (1) has satisfactory corrosion resistance and a high pickup suppression effect.

It is preferable that the numbers of polar groups included in R¹ and R⁴ are each 2 or more, even in a case where both R¹ and R⁴ represent an end group represented by Formula (2) or in a case where one of R¹ or R⁴ represents an end group that does not correspond to Formula (2). In a case where the numbers of polar groups included in R¹ and R⁴ are each 2 or more, the number of polar groups which can interact with the protective layer and the number of polar groups which can be involved in the intermolecular interaction increase, and thus the fluorine-containing ether compound can form a lubricating layer having more satisfactory corrosion resistance and a higher pickup suppression effect.

In addition, the numbers of polar groups included in R¹ and R⁴ are each 4 or less. Therefore, in the lubricating layer containing the fluorine-containing ether compound, the aggregation of the polar groups of the end group represented by R¹ and R⁴ can be effectively suppressed. Therefore, the fluorine-containing ether compound aggregates and forms a lump, can suppress the smoothness of the lubricating layer from being lost, and thus can suppress occurrence of the pickup due to the collision between the lubricating layer and the magnetic head. In addition, since the number of polar groups included in R¹ and R⁴ is 4 or less, a polar group which is not involved in the interaction with the active point on the protective layer or the interaction with the polar group contained in another fluorine-containing ether compound present in the lubricating layer is unlikely to be generated in R¹ and R⁴. As a result, in a case where the polar group which is not involved in these interactions is present in R¹ and R⁴, it is possible to prevent water, which causes corrosion, from being taken into the magnetic recording medium including the lubricating layer containing the fluorine-containing ether compound due to an extreme increase in hydrophilicity of the fluorine-containing ether compound. Therefore, in a case where the number of polar groups included in R¹ and R⁴ is 4 or less, the fluorine-containing ether compound can form a lubricating layer having high corrosion resistance and a high pickup suppression effect. The numbers of polar groups included in R¹ and R⁴ are each preferably 3 or less.

The total number of polar groups included in R¹ and R⁴ in Formula (1) is 3 or more and more preferably 4 or more. Since the total number of the above-described polar groups is 3 or more, a polar group which can interact with the protective layer and a polar group which can be involved in the intermolecular interaction is obtained by the polar group included in R¹ and R⁴. As a result, the lubricating layer containing the fluorine-containing ether compound represented by Formula (1) has excellent adhesion to the protective layer, satisfactory corrosion resistance, and a high pickup suppression effect. The total number of polar groups included in R¹ and R⁴ in Formula (1) is preferably 6 or less. In a case where the total number of the above-described polar groups is 6 or less, the polarity of the fluorine-containing ether compound is extremely high, and thus it is possible to more effectively prevent water, which causes corrosion, from being taken into the magnetic recording medium including the lubricating layer that contains the fluorine-containing ether compound.

The polar group contained in the end group which does not correspond to Formula (2) in a case where one of R¹ and R⁴ represents an end group which does not correspond to Formula (2), and the polar group which may be included in X in Formula (2) are preferably at least one polar group selected from the group consisting of a hydroxy group (—OH), a carboxy group (—COOH), a formyl group (—(C═O)H), a carbonyl group (—(C═O)R⁷; R⁷ represents an organic group), a sulfo group (—SO₃H), a cyano group (—CN), a group having an amide bond (—NR⁸COR⁹ or —CONR¹⁰R¹¹; R⁸, R⁹, R¹⁰, and R¹¹ each independently represent a hydrogen atom or an organic group), and an amino group (—NR¹²R¹³; R¹² and R¹³ each independently represent a hydrogen atom or an organic group). The group having an amide bond includes, as shown in the formula above, both a group (for example, a carboxamide group (—C(═O)NH₂)) bonded to a carbon atom constituting the amide bond and a group (for example, an acetamido group (—NHC(═O)CH₃)) bonded to a nitrogen atom constituting the amide bond. In the group having an amide bond, R⁸ and R⁹ may be bonded to each other to form a ring, and R¹⁰ and R¹¹ may be bonded to each other to form a ring. It is preferable that R⁸, R⁹, R¹⁰, and R¹¹ in the group having an amide bond are each independently selected from the group consisting of a hydrogen atom, a methyl group, an ethyl group, a propyl group, and a butyl group.

The term “polar group” in the present specification does not include a halogeno group (—F, —Cl, —Br, or the like) or an ether bond (—O—).

In a case where one of R¹ or R⁴ represent an end group which does not correspond to Formula (2), it is preferable that the polar group contained in the end group which does not correspond to Formula (2) and the polar group which may be included in X in Formula (2) include at least one polar group selected from the group consisting of a hydroxy group, a cyano group, and a group having an amide bond. The reason for this is that the hydroxy group, the cyano group, and the group having an amide bond are chemically stable, and a lubricating layer containing a fluorine-containing ether compound having these polar groups does not deteriorate for a long period of time. In addition, it is because the acidity of the hydroxy group, the cyano group, and the group having an amide bond is not extremely high, and thus the substrate is unlikely to be corroded.

In a case where one of R¹ or R⁴ represents an end group that does not correspond to Formula (2), it is preferable that the end group that does not correspond to Formula (2) contains at least one hydroxy group. This is because the state of coating the protective layer with the fluorine-containing ether compound is more uniform.

It is more preferable that, even in a case where one of R¹ or R⁴ represents an end group represented by Formula (2) or in a case where both of R¹ and R⁴ represent an end group represented by Formula (2), the polar group included in R¹ and the polar group included in R⁴ are all hydroxy groups. This is because the state of coating the protective layer with the fluorine-containing ether compound is more uniform.

The polar group included in the end group that does not correspond to Formula (2) in a case where one of R¹ or R⁴ represents an end group that does not correspond to Formula (2) and the polar group which may be included in X in Formula (2) may be partially or entirely the same as or different from each other.

In addition, the number of polar groups included in R¹ and the number of polar groups included in R⁴ may be the same as or different from each other. It is preferable that the number of polar groups included in R¹ and the number of polar groups included in R⁴ are the same as each other because the state of coating the protective layer with the fluorine-containing ether compound is more uniform so that a lubricating layer having more satisfactory adhesion can be formed.

In a case where one of R¹ or R⁴ represents an end group that does not correspond to Formula (2), the number of carbon atoms of the end group that does not correspond to Formula (2) in R¹ and R⁴ is 1 or more. In addition, the end group represented by Formula (2) has at least two carbon atoms having a 1,2-diol structure and two or more carbon atoms in X, and has a total of 4 or more carbon atoms. Since the number of carbon atoms in the end group represented by R¹ and R⁴ is 1 or more, the hydrophobicity of the end group can be ensured, attraction of water, which causes corrosion, to the lubricating layer is prevented, and thus a lubricating layer having satisfactory corrosion resistance can be formed. In a case where one of R¹ or R⁴ represents an end group that does not correspond to Formula (2), the number of carbon atoms of the end group that does not correspond to Formula (2) in R¹ and R⁴ is preferably 3 or more and more preferably 4 or more.

In a case where one of R¹ or R⁴ represents an end group that does not correspond to Formula (2), the number of carbon atoms of the end group that does not correspond to Formula (2) in R¹ and R⁴ is 50 or less. In addition, the end group represented by Formula (2) includes two carbon atoms having a 1,2-diol structure and 30 or less carbon atoms of X, and has a total of 32 or less carbon atoms. Since the number of carbon atoms in the end group represented by R¹ and R⁴ is each 50 or less, the end group has a flexible structure, and thus adhesion between the lubricating layer and the protective layer containing the fluorine-containing ether compound is improved. As a result, a lubricating layer capable of suppressing the pickup can be formed. The numbers of carbon atoms of the end group represented by R¹ and R⁴ are each preferably 20 or less and more preferably 15 or less.

### [End Group Represented by Formula (2)]

At least one of R¹ or R⁴ represents an end group represented by Formula (2).

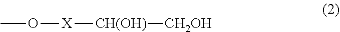

(In Formula (2), X represents a divalent organic group having 2 to 30 carbon atoms, which may have at least one of one or two polar groups or one to three ether oxygen atoms. X has at least one carbon atom which is not bonded to either the polar groups or the ether oxygen atoms.)

The end group represented by Formula (2) has an oxygen atom bonded to a methylene group (—CH₂—) bonded to R². Oxygen atoms disposed at the ends of the end groups represented by Formula (2) form an ether bond (—O—) with atoms bonded to both sides of the oxygen atoms. This ether bond imparts moderate flexibility to the fluorine-containing ether compound represented by Formula (1), and increases the affinity between the polar group of the end group represented by Formula (2) and the protective layer. In this manner, the fluorine-containing ether compound represented by Formula (1) can form a lubricating layer having excellent adhesion to the protective layer.

The end group represented by Formula (2) has a 1,2-diol structure, and a hydroxy group which can interact with the protective layer and a hydroxy group which can be involved in the intermolecular interaction can be ensured while a decrease in hydrophobicity due to the hydroxy group contained in the 1,2-diol structure is suppressed as described above.

Therefore, the lubricating layer containing the fluorine-containing ether compound represented by Formula (1) has a low affinity for water so that water, which causes corrosion, is unlikely to be taken into the layer, and thus the corrosion resistance is excellent. In addition, since the polar group that is not involved in the interaction with the protective layer easily forms the intermolecular interaction, the fluorine-containing ether compound in the lubricating layer is unlikely to move from the protective layer to the magnetic head, and thus the pickup is suppressed.

X in the end group represented by Formula (2) has at least one carbon atom that is not bonded to either the polar group or the ether oxygen atom. Therefore, the lubricating layer containing the fluorine-containing ether compound represented by Formula (1) has a low affinity for water. Further, the carbon atom imparts moderate rigidity to the fluorine-containing ether compound molecule and suppresses the intramolecular interaction between the polar groups, and thus the polar groups are likely to be involved in the intermolecular interaction between the fluorine-containing ether compounds. Therefore, the above-described carbon atom included in X of the end group represented by Formula (2) contributes to the formation of the lubricating layer having satisfactory corrosion resistance and a high pickup suppression effect.

Meanwhile, in a case where, for example, —OCH₂CH(OH)CH₂OCH₂— is disposed instead of —O—X— in Formula (2), all carbon atoms in the structure are bonded to the polar group or the ether oxygen atom. The lubricating layer containing such a fluorine-containing ether compound has a higher affinity for water and degraded corrosion resistance as compared with the lubricating layer containing the fluorine-containing ether compound represented by Formula (1), and in addition, since the polar group which can be involved in the intermolecular interaction is insufficient, the pickup resistance is degraded.

Since the organic group represented by X in Formula (2) has 2 or more carbon atoms, the organic group imparts moderate rigidity and suppresses the intramolecular interaction between the polar groups so that the polar group is likely to be involved in the intermolecular interaction between the fluorine-containing ether compounds, and the interaction with water is hindered, and thus the hydrophobicity is improved. In addition, since the number of carbon atoms in the organic group represented by X is 30 or less, the end group represented by Formula (2) is not extremely bulky, and the movement of the polar group contained in the fluorine-containing ether compound is not hindered so that the inhibition of the interaction between the polar group and the protective layer can be suppressed. The number of carbon atoms in the organic group represented by X is preferably in a range of 2 to 15 and more preferably in a range of 3 to 10.

The organic group represented by X has at least one carbon atom that is not bonded to either the polar group or the ether oxygen atom. Examples of the carbon atom that is not bonded to any of the polar group and the ether oxygen atom include a carbon atom of a methylene group, a methine group, and groups in which these groups are fluorinated, which are not bonded to any of the polar group and the ether oxygen atom. As described above, the polar group does not include a fluoro group.

In a case where the organic group represented by X has a polar group having a carbon atom (for example, a carboxy group, a formyl group, a carbonyl group, a cyano group, or a group having an amide bond), the carbon atom of the polar group is not included in “carbon atom that is not bonded to any of the polar group and the ether oxygen atom”.

The number of carbon atoms that are not bonded to any of the polar group and the ether oxygen atom may be 1 or more, and may be, for example, 1 to 6 or 1 to 4. In a case where the number of carbon atoms that are not bonded to any of the polar group and the ether oxygen atom is 1 or more, hydrophobicity of the lubricating layer containing the fluorine-containing ether compound is enhanced. In addition, in a case where the number of carbon atoms that are not bonded to any of the polar group and the ether oxygen atom is 6 or less, the ratio of the number of carbon atoms to the number of polar groups is appropriate, and the polarity of the molecules is appropriate, the ratio of the number of carbon atoms to the number of ether oxygen atoms is appropriate, and thus a fluorine-containing ether compound in which the flexibility of the molecules is appropriate is obtained. In addition, in a case where the compound has a plurality of carbon atoms that are not bonded to any of the polar groups and the ether oxygen atoms, the carbon atoms that are not bonded to any of the polar groups and the ether oxygen atoms may be bonded to each other. In this case, the fluorine-containing ether compound molecules are more rigid, and the intramolecular interaction between the polar groups is suppressed so that the polar groups are more likely to be further involved in the intermolecular interaction between the fluorine-containing ether compounds.

The organic group represented by X in Formula (2) may contain one or two polar groups. In a case where the organic group represented by X in Formula (2) contains one or two polar groups, the adhesion of the fluorine-containing ether compound represented by Formula (1) to the protective layer is improved, and a lubricating layer in which the coating state can be sufficiently obtained even in a case of reducing the thickness is likely to be formed. In a case where the organic group represented by X in Formula (2) contains a polar group, the number of polar groups is preferably 1. This is because, in a case where the hydrophilicity of the fluorine-containing ether compound represented by Formula (1) is not extremely high, and thus attraction of water, which causes corrosion, can be suppressed.

As the polar group in a case where the organic group represented by X in Formula (2) has a polar group, a polar group selected from the group consisting of a hydroxy group, a cyano group, and a group having an amide bond is preferable.

The organic group represented by X in Formula (2) may have 1 to 3 ether bonds. In a case where the organic group represented by X in Formula (2) has an ether bond, an oxygen atom forming the ether bond imparts moderate flexibility to the fluorine-containing ether compound represented by Formula (1), and increases the affinity between the polar group and the protective layer. In a case where the organic group represented by X in Formula (2) has an ether bond, the number of the ether bonds is preferably 1 or 2. This is because the fluorine-containing ether compound represented by Formula (1) is not extremely flexible so that a polar group involved in the intramolecular interaction is suppressed from being easily generated.

The organic group represented by X in Formula (2) may be partially fluorinated. In a case where the organic group represented by X in Formula (2) is partially fluorinated, the hydrophilicity of the organic group represented by X in Formula (2) is decreased as compared with a case where the organic group represented by X is not fluorinated. As a result, attraction of water, which causes corrosion, can be suppressed, and thus a fluorine-containing ether compound capable of forming a lubricating layer having more satisfactory corrosion resistance is obtained.

It is preferable that the organic group represented by X in Formula (2) is an acyclic organic group. In a case where the organic group represented by X is acyclic, the organic group represented by X is not extremely bulky as compared with a case where X represents an organic group having a cyclic structure. Therefore, the movement of the polar group contained in the fluorine-containing ether compound is not hindered by the organic group represented by X so that the inhibition of the interaction between the polar group and the protective layer can be suppressed. The acyclic organic group may be linear or branched.

It is preferable that the organic group represented by X in Formula (2) does not have an unsaturated carbon-carbon bond. In a case where the organic group represented by X does not have an unsaturated carbon-carbon bond, the alignment of the molecules of the organic group represented by X is not limited and the movement of the polar group contained in the fluorine-containing ether compound is not hindered so that inhibition of the interaction between the polar group and the protective layer can be suppressed as compared with a case where X has an unsaturated carbon-carbon bond.

More specifically, it is preferable that the end group represented by Formula (2) is an end group represented by any of Formulae (2-1) to (2-7).

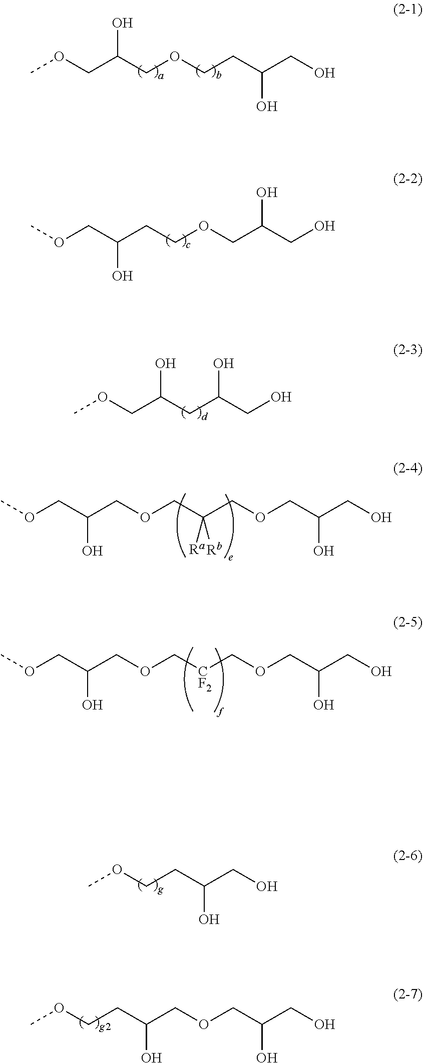

(In Formula (2-1), a represents an integer of 1 to 8. b represents an integer of 1 to 7.)

(In Formula (2-2), c represents an integer of 1 to 7.)

(In Formula (2-3), d represents an integer of 1 to 6.)

(In Formula (2-4), e represents an integer of 1 to 6. e pieces of R^{a}'s and R^{b}'s each independently represent a hydrogen atom or a methyl group.)

(In Formula (2-5), f represents an integer of 1 to 6.)

(In Formula (2-6), g represents an integer of 1 to 6.)

(In Formula (2-7), g2 represents an integer of 1 to 6.)

The end group represented by Formula (2-1) is an end group in which —X— in Formula (2) is —CH₂—CH(OH)—(CH₂)ₐ—O—(CH₂)_{b}—CH₂—. a represents an integer of 1 to 8, and b represents an integer of 1 to 7.

The end group represented by Formula (2-1) has a linking group in which (b+1) pieces of methylene groups are bonded between a 1,2-diol structure (—CH(OH)—CH₂OH) disposed at the end of Formula (2) and an ether oxygen atom forming an ether bond of X in Formula (2). The linking group has b pieces of carbon atoms that are not bonded to any of the polar group and the ether oxygen atom. Therefore, the end group represented by Formula (2-1) has moderate rigidity due to the linking group consisting of (b+1) pieces of methylene groups, and thus the hydroxy group contained in the end group represented by Formula (2-1) can be suppressed from forming an intramolecular interaction.

Since b in Formula (2-1) represents 1 or greater, the end group represented by Formula (2-1) has a carbon atom that is not bonded to any of the polar group and the ether oxygen atom, and thus hydrophobicity is sufficiently obtained. In addition, since b represents 7 or less, the end group represented by Formula (2-1) is not extremely bulky, and the movement of the hydroxy group contained in the end group represented by Formula (2-1) is not hindered so that inhibition of the interaction between the hydroxy group and the protective layer can be suppressed. b represents preferably 1 to 5 and more preferably 1 to 3.

In addition, the end group represented by Formula (2-1) has a linking group in which a pieces of methylene groups are bonded between a carbon atom to which a secondary hydroxy group on the perfluoropolyether chain side is bonded and an ether oxygen atom forming an ether bond. Since a in Formula (2-1) represents 1 or greater, the end group represented by Formula (2-1) has sufficient hydrophobicity. In addition, since a represents 8 or less, the end group represented by Formula (2-1) is not extremely bulky, and the movement of the hydroxy group contained in the end group represented by Formula (2-1) is not hindered so that inhibition of the interaction between the hydroxy group and the protective layer can be suppressed. a represents preferably 1 to 6 and more preferably 1 to 4.

It is preferable that the end group represented by Formula (2-1) has a sum of a and b of 9 or less. In a case where the sum of a and b is 9 or less, the end group represented by Formula (2-1) is not extremely bulky, and the movement of the hydroxy group contained in the end group represented by Formula (2-1) is not hindered so that inhibition of the interaction between the hydroxy group and the protective layer can be effectively suppressed. The sum of a and b is more preferably 6 or less.

In addition, since X in Formula (2) has an ether bond, the end group represented by Formula (2-1) has moderate mobility. Therefore, in a case where only one hydroxy group between two hydroxy groups of the 1,2-diol structure in Formula (2-1) interacts with the protective layer, the other hydroxy group is more likely to interact with the polar group of the other fluorine-containing ether compound. Therefore, the hydroxy group in Formula (2-1) can easily form an intermolecular interaction. Therefore, the lubricating layer containing the fluorine-containing ether compound having an end group represented by Formula (2-1) is difficult to move from the protective layer to the magnetic head, and thus the pickup is suppressed.

The end group represented by Formula (2-2) is an end group in which —X— in Formula (2) is —CH₂—CH(OH)—CH₂—(CH₂)_{c}—O—CH₂—. c represents an integer of 1 to 7. The end group represented by Formula (2-2) has a linking group in which (c+1) pieces methylene groups are bonded between a carbon atom to which a secondary hydroxy group on the perfluoropolyether chain side is bonded and an ether oxygen atom forming an ether bond of X in Formula (2). The linking group has c pieces of carbon atoms that are not bonded to any of the polar group and the ether oxygen atom. Therefore, the end group represented by Formula (2-2) has moderate rigidity due to the linking group consisting of (c+1) pieces of methylene groups, and the hydroxy group contained in the end group represented by Formula (2-2) can be suppressed from forming an intramolecular interaction.

In addition, since c in Formula (2-2) represents 1 or greater, the end group represented by Formula (2-2) has a carbon atom that is not bonded to any of the polar group and the ether oxygen atom, and thus sufficient hydrophobicity is obtained. In addition, since c represents 7 or less, the end group represented by Formula (2-2) is not extremely bulky, and the movement of the hydroxy group contained in the end group represented by Formula (2-2) is not hindered so that inhibition of the interaction between the hydroxy group and the protective layer can be suppressed. c represents preferably 1 to 5 and more preferably 1 to 3.

In addition, the end group represented by Formula (2-2) is an end group in which X in Formula (2) has an ether bond, and since an ether oxygen atom forming the ether bond is bonded to a carbon atom to which a 1,2-diol structure (—CH(OH)—CH₂OH) is bonded, the end group has high mobility. Therefore, only one hydroxy group between two hydroxy groups of the 1,2-diol structure in Formula (2-2) is likely to interact with the protective layer, the other hydroxy group is likely to interact with the polar group of the other fluorine-containing ether compound. Accordingly, the hydroxy group in Formula (2-2) can easily form an intermolecular interaction. Therefore, the lubricating layer containing the fluorine-containing ether compound having an end group represented by Formula (2-2) is difficult to move from the protective layer to the magnetic head, and thus the pickup is suppressed.

The end group represented by Formula (2-3) is an end group in which —X— in Formula (2) is —CH₂—CH(OH)—(CH₂)ₐ—. d represents an integer of 1 to 6. The end group represented by Formula (2-3) has a linking group in which d pieces methylene groups are bonded between a 1,2-diol structure (—CH(OH)—CH₂OH) and a carbon atom to which the secondary hydroxy group of X in Formula (2) is bonded. The linking group has d pieces of carbon atoms that are not bonded to any of the polar group and the ether oxygen atom. Therefore, the end group represented by Formula (2-3) has moderate rigidity due to the linking group consisting of d pieces of methylene groups, and the hydroxy group contained in the end group represented by Formula (2-3) can be suppressed from forming an intramolecular interaction.

Since d in Formula (2-3) represents 1 or greater, the end group represented by Formula (2-3) has a carbon atom that is not bonded to any of the polar group and the ether oxygen atom, and thus hydrophobicity is sufficiently obtained. In addition, since d represents 6 or less, the end group represented by Formula (2-3) is not extremely bulky, and the movement of the hydroxy group contained in the end group represented by Formula (2-3) is not hindered so that inhibition of the interaction between the hydroxy group and the protective layer can be suppressed. d represents preferably 1 to 4 and more preferably 2 to 4.

In addition, the end group represented by Formula (2-3) does not have an ether bond in which X in Formula (2) is a hydrophilic moiety. Therefore, the end group represented by Formula (2-3) has a low affinity for water and satisfactory hydrophobicity. Therefore, the fluorine-containing ether compound having the end group represented by Formula (2-3) can form a lubricating layer having more satisfactory corrosion resistance because water, which causes corrosion, is unlikely to be taken into the layer.

The end group represented by Formula (2-4) is an end group in which —X— in Formula (2) is —CH₂—CH(OH)—CH₂—O—CH₂—(CR^{a}R^{b})_{c}—CH₂—O—CH₂—. e represents an integer of 1 to 6, and e pieces of R^{a}'s and R^{b}'s each independently represent a hydrogen atom or a methyl group. That is, e pieces of (—CR^{a}R^{b}—)'s in an end group represented by Formula (2-4) may each be any of —CH₂—, —CH(CH₃)—, or —C(CH₃)₂—.

The end group represented by Formula (2-4) is an end group in which X in Formula (2) has two ether bonds, and has a linking group having a group (—CR^{a}R^{b}—) having at least e pieces of carbon atoms that are not bonded to any of the polar groups and the ether oxygen atoms between oxygen atoms forming two ether bonds.

Since e in Formula (2-4) represents 1 or greater, the end group represented by Formula (2-4) has a carbon atom that is not bonded to any of the polar group and the ether oxygen atom, and thus hydrophobicity is sufficiently obtained. In addition, since e represents 6 or less, the end group represented by Formula (2-4) is not extremely bulky, and the movement of the hydroxy group contained in the end group represented by Formula (2-4) is not hindered so that inhibition of the interaction between the hydroxy group and the protective layer can be suppressed. e represents preferably 1 to 4 and more preferably 1 or 2.

In a case where X in Formula (2) in the end group represented by Formula (2-4) has a linking group having a linear structure between oxygen atoms forming two ether bonds (in other words, e pieces of R^{a}'s and R^{b}'s are all hydrogen atoms), the end group represented by Formula (2-4) is not extremely bulky so that inhibition of the movement of the hydroxy group in the end group represented by Formula (2-4) can be suppressed as compared with a case where X has a linking group having a branched structure between the oxygen atoms (in other words, one or more of e pieces of R^{a}'s and R^{b}'s represent a methyl group). Therefore, in a case where X in Formula (2) in the end group represented by Formula (2-4) has a linking group having a linear structure between oxygen atoms forming two ether bonds, only one hydroxy group of the two hydroxy groups in the 1,2-diol structure in Formula (2-4) is likely to interact with the protective layer, and the other hydroxy group is likely to interact with the polar group in the other fluorine-containing ether compound. Therefore, the hydroxy group in Formula (2-4) can easily form intermolecular interaction. Therefore, the lubricating layer containing the fluorine-containing ether compound having an end group represented by Formula (2-4) is difficult to move from the protective layer to the magnetic head, and thus the pickup is suppressed.

In a case where X in Formula (2) in the end group represented by Formula (2-4) has a linking group having a branched structure between the oxygen atoms forming two ether bonds (in other words, in a case of including —CH(CH₃)— and/or —C(CH₃)₂—), X is moderately rigid. Therefore, the hydroxy groups in the end group represented by Formula (2-4) can be effectively suppressed from forming an intramolecular interaction, and the hydroxy groups in Formula (2-4) can easily form an intermolecular interaction. Therefore, the lubricating layer containing the fluorine-containing ether compound having an end group represented by Formula (2-4) is difficult to move from the protective layer to the magnetic head, and thus the pickup resistance is excellent.

The end group represented by Formula (2-5) is an end group in which —X— in Formula (2) is —CH₂—CH(OH)—CH₂—O—CH₂—(CF₂)_{f}—CH₂—O—CH₂—. f represents an integer of 1 to 6.

The end group represented by Formula (2-5) is an end group in which X in Formula (2) has two ether bonds, and has a linking group having f pieces of carbon atoms that are not bonded to any of the polar groups and the ether oxygen atoms between oxygen atoms forming two ether bonds.

The end group represented by Formula (2-5) has a linear perfluoroalkylene chain (—(CF₂)_{f}—) having 1 to 6 carbon atoms. The linear perfluoroalkylene chain having 1 to 6 carbon atoms has excellent hydrophobicity, and reduces the affinity of the lubricating layer containing the fluorine-containing ether compound for water.

The end group represented by Formula (2-5) has a linking group consisting of a saturated hydrocarbon group (—CH₂—(CF₂)_{f}—CH₂— in Formula (2-5)) having a linear perfluoroalkylene chain having 1 to 6 carbon atoms between the oxygen atoms forming two ether bonds. Therefore, the polarity of the entire fluorine-containing ether compound molecules is low, and thus a lubricating layer having satisfactory hydrophobicity can be formed. As a result, since the lubricating layer containing the fluorine-containing ether compound having an end group represented by Formula (2-5) has a low affinity for water, which causes corrosion, water is unlikely to be taken into the layer, and thus the corrosion resistance is further enhanced.

Since f in Formula (2-5) represents 1 or greater, the end group represented by Formula (2-5) has a carbon atom that is not bonded to any of the polar group and the ether oxygen atom, hydrophobicity is sufficiently obtained, the end group is moderately rigid, and thus the hydroxy groups in the end group represented by Formula (2-5) can be suppressed from forming an intramolecular interaction. In addition, since f represents 6 or less, the end group represented by Formula (2-5) is not extremely bulky, and the movement of the hydroxy group contained in the end group represented by Formula (2-5) is not hindered so that inhibition of the interaction between the hydroxy group and the protective layer can be suppressed. f represents preferably 1 to 4 and more preferably 2 to 4.

The end group represented by Formula (2-6) is an end group in which —X— in Formula (2) is —(CH₂)_{g}—CH₂—. g represents an integer of 1 to 6.

The end group represented by Formula (2-6) has a linking group in which (g+1) pieces methylene groups are bonded between a 1,2-diol structure (—CH(OH)—CH₂OH) and an ether oxygen atom of a bonding end. In a case where the end group has the linking group, the end group represented by Formula (2-6) has g pieces of carbon atoms that are not bonded to any of the polar group and the ether oxygen atom. Therefore, the end group represented by Formula (2-6) has moderate rigidity due to the linking group consisting of (g+1) pieces of methylene groups, and the hydroxy group contained in the end group represented by Formula (2-6) can be suppressed from forming an intramolecular interaction.

Since g in Formula (2-6) represents 1 or greater, the end group represented by Formula (2-6) has a carbon atom that is not bonded to any of the polar group and the ether oxygen atom, the end group is moderately rigid, and thus the hydroxy groups in the 1,2-diol structure can be suppressed from forming an intramolecular interaction. In addition, since g in Formula (2-6) represents 6 or less, the end group represented by Formula (2-6) is not extremely bulky, and the movement of the hydroxy group having a 1,2-diol structure is not hindered so that inhibition of the interaction between the hydroxy group and the protective layer can be suppressed. g represents preferably 1 to 5 and more preferably 1 to 3.

In addition, the end group represented by Formula (2-6) does not have a polar group and an ether bond in which X in Formula (2) is a hydrophilic moiety. Therefore, the end group represented by Formula (2-6) has a low affinity for water and satisfactory hydrophobicity. Therefore, the fluorine-containing ether compound having the end group represented by Formula (2-6) can form a lubricating layer having more satisfactory corrosion resistance because water, which causes corrosion, is unlikely to be taken into the layer.

The end group represented by Formula (2-7) is an end group in which the organic group represented by X in Formula (2) is —(CH₂)_{g2}—CH₂—CH(OH)—CH₂—O—CH₂—. g2 represents an integer of 1 to 6.

The end group represented by Formula (2-7) has a glycerin structure (—O—CH₂—CH(OH)—CH₂OH) at an end portion. Since the glycerin structure has high mobility, in a case where one hydroxy group of the two hydroxy groups at the end portion interacts with the protective layer, the other hydroxy group is likely to interact with the polar group in the other fluorine-containing ether compound between molecules. Therefore, the polar group in the fluorine-containing ether compound represented by Formula (1) can more easily form an intermolecular interaction. In this manner, the lubricant containing the fluorine-containing ether compound represented by Formula (1) is difficult to move from the protective layer to the magnetic head, and has excellent pickup resistance.

Since g2 in Formula (2-7) represents 1 or greater, the end group represented by Formula (2-7) is moderately rigid, and the hydroxy group having a 1,2-diol structure is unlikely to form an intramolecular interaction. In addition, since g2 in Formula (2-7) represents 6 or less, the end group represented by Formula (2-7) is not extremely bulky, and the movement of the hydroxy group is not hindered so that inhibition of the interaction between the hydroxy group and the protective layer can be suppressed. g2 represents preferably 1 to 5 and more preferably 1 to 3.

R¹ and R⁴ in Formula (1) may each independently represent an end group represented by Formula (2), or only one of R¹ or R⁴ may represent an end group represented by Formula (2).

In addition, in the fluorine-containing ether compound represented by Formula (1), R¹ and R⁴ may be the same as or different from each other. It is preferable that R¹ and R⁴ are the same as each other. This is because the compound is a fluorine-containing ether compound which can be easily and efficiently produced.

Further, the expression “R¹ and R⁴ are the same as each other” denotes that the atom in R¹ and the atom in R⁴ are symmetrically disposed with respect to —CH₂—R²[—CH₂—R³—CH₂—R²]ₓ—CH₂—.

In the fluorine-containing ether compound represented by Formula (1), in a case where R¹ and R⁴ are different from each other, both R¹ and R⁴ may represent an end group represented by Formula (2), or one of R¹ or R⁴ may represent an end group represented by Formula (2) and the other may represent an end group that does not correspond to Formula (2). The end group that does not correspond to Formula (2) may be an end group having one to four polar groups and 1 to 50 carbon atoms as described above.

### [End Group Represented by Formula (3)]

In a case where one of R¹ or R⁴ represents an end group represented by Formula (2) and the other represents an end group that does not correspond to Formula (2), it is preferable that the other is an end group that is represented by Formula (3) and does not correspond to Formula (2).

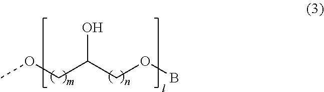

(In Formula (3), l represents an integer of 1 to 3. l pieces of m's each independently represent an integer of 1 to 6. l pieces of n's each independently represent an integer of 1 to 6. In one repeating unit, at least one of m or n represents 1. B represents an alkyl group which may have only one polar group, an organic group having one or more carbon-carbon unsaturated bonds, or a hydrogen atom.)

The end group represented by Formula (3) has an oxygen atom bonded to a methylene group (—CH₂—) bonded to R². Oxygen atoms disposed at the ends of the end group represented by Formula (3) form an ether bond (—O—) with atoms bonded to both sides of the oxygen atoms. This ether bond imparts moderate flexibility to the fluorine-containing ether compound represented by Formula (1), and increases the affinity between the polar group of the end group represented by Formula (3) and the protective layer. In this manner, the fluorine-containing ether compound represented by Formula (1) can form a lubricating layer having excellent adhesion to the protective layer.

l in Formula (3) represents an integer of 1 to 3, preferably an integer of 1 or 2, and most preferably 1. In a case where 1 in Formula (3) represents 3 or less, the amount of the hydroxy group of the end group represented by Formula (3) is extremely large, and thus water, which causes corrosion, can be prevented from being attracted to the lubricating layer. Therefore, a lubricating layer with satisfactory corrosion resistance can be obtained.

In a case where 1 in Formula (3) represents 2 or 3, combinations of m's and n's in two or three repeating units (—(CH₂)ₘ—CH(OH)—(CH₂)ₙ—O—) may be partially or entirely the same as or different from each other.

l pieces of m's in Formula (3) each independently represent an integer of 1 to 6, and l pieces of n's each independently represent an integer of 1 to 6.

In one repeating unit (—(CH₂)ₘ—CH(OH)—(CH₂)ₙ—O—) of Formula (3), at least one of m or n represents 1. This is because the mobility of the hydroxy group in the repeating unit is not reduced due to an extreme increase in the number of carbon atoms in the alkylene group between the carbon atom to which the hydroxy group is bonded and the ether oxygen atom.

It is preferable that I pieces of m's in Formula (3) each independently represent an integer of 1 to 4. In addition, it is preferable that l pieces of n's each independently represent an integer of 1 to 4.

B in Formula (3) represents an alkyl group which may have only one polar group, an organic group having one or more carbon-carbon unsaturated bonds, or a hydrogen atom.

In a case where B in Formula (3) represents an alkyl group having no polar group, specific examples of B include a methyl group, an ethyl group, a propyl group, a butyl group, a pentyl group, and a hexyl group.

In a case where B in Formula (3) represents an alkyl group having a polar group, the polar group is preferably any of the preferred examples of the polar group included in the end group not corresponding to Formula (2). Among the above-described polar groups, a polar group selected from the group consisting of a hydroxy group, a cyano group, or a group having an amide bond is more preferable.

In a case where B in Formula (3) represents an alkyl group having a polar group, examples of B include a 2-hydroxyethyl group, a 3-hydroxypropyl group, a 4-hydroxybutyl group, a 5-hydroxypentyl group, a 6-hydroxyhexyl group, a 2-aminoethyl group, a 3-aminopropyl group, a 1-carboxymethyl group, a 2-carboxyethyl group, a 3-carboxypropyl group, a 1-carbonylmethyl group, a 2-carbonylethyl group, a 3-carbonylpropyl group, a 1-acetylmethyl group, a 2-acetylethyl group, a 3-acetylpropyl group, a 2-sulfoethyl group, a 3-sulfopropyl group, a 1-cyanomethyl group, a 2-cyanoethyl group, a 3-cyanopropyl group, a 4-cyanobutyl group, a 2-acetoamidoethyl group, a 3-acetoamidopropyl group, a 4-acetoamidobutyl group, a 1-carboxamidomethyl group, a 2-carboxamidoethyl group, a 3-carboxamidopropyl group, and a 4-carboxamidobutyl group.

Among the alkyl groups having the above-described polar group, B represents preferably any of a 2-hydroxyethyl group, a 3-hydroxypropyl group, a 4-hydroxybutyl group, a 5-hydroxypentyl group, a 6-hydroxyhexyl group, a 1-cyanomethyl group, a 2-cyanoethyl group, a 3-cyanopropyl group, a 2-acetoamidoethyl group, a 1-carboxamidomethyl group, a 2-carboxamidoethyl group, or a 3-carboxamidopropyl group and more preferably any of a 2-hydroxyethyl group, a 3-hydroxypropyl group, a 2-cyanoethyl group, a 3-cyanopropyl group, a 2-acetoamidoethyl group, or a 1-carboxamidomethyl group.

In a case where B in Formula (3) represents an organic group having one or more carbon-carbon unsaturated bonds, examples of B include an organic group having at least one selected from an aromatic hydrocarbon, an unsaturated heterocycle, an alkenyl group, and an alkynyl group.

In a case where B in Formula (3) represents an organic group having one or more carbon-carbon unsaturated bonds, examples of B include a phenyl group, a methoxyphenyl group, a fluorinated phenyl group, an acetamidophenyl group, a carboxamidophenyl group, a cyanophenyl group, a naphthyl group, a phenethyl group, a methoxyphenethyl group, a fluorinated phenethyl group, a benzyl group, a methoxybenzyl group, a naphthylmethyl group, a methoxynaphthyl group, a pyrrolyl group, a pyrazolyl group, a methylpyrazolylmethyl group, an imidazolyl group, a furyl group, a furfuryl group, an oxazolyl group, an isoxazolyl group, a thienyl group, a thienylethyl group, a thiazolyl group, a methylthiazolylethyl group, an isothiazolyl group, a pyridyl group, a pyrimidinyl group, a pyridazinyl group, a pyrazinyl group, an indolinyl group, a benzofuranyl group, a benzothienyl group, a benzimidazolyl group, a benzoxazolyl group, a benzothiazolyl group, a benzopyrazolyl group, a benzoisoxazolyl group, a benzoisothiazolyl group, a quinolyl group, an isoquinolyl group, a quinazolinyl group, a quinoxalinyl group, a phthalazinyl group, a cinnolinyl group, a vinyl group, an allyl group, a butenyl group, a propynyl group, a propargyl group, a butynyl group, a methylbutynyl group, a pentynyl group, a methylpentynyl group, and a hexynyl group.

Among the above-described organic groups having one or more carbon-carbon unsaturated bonds, B represents preferably any of a phenyl group, a methoxyphenyl group, an acetamidophenyl group, a carboxamidophenyl group, a cyanophenyl group, a naphthyl group, a phenethyl group, a methoxyphenethyl group, a fluorinated phenethyl group, a thienylethyl group, an allyl group, a butenyl group, or a propargyl group and particularly preferably any of a phenyl group, a methoxyphenyl group, a carboxamidophenyl group, an allyl group, or a butenyl group. This is because the organic group has sufficient hydrophobicity, is not extremely bulky, and the movement of the hydroxy group in Formula (3) is not hindered so that inhibition of the interaction between the hydroxy group and the protective layer can be suppressed.

In a case where B in Formula (3) represents a hydrogen atom, B forms a hydroxy group with the oxygen atom in Formula (3).

In a case where one of R¹ or R⁴ represents an end group represented by Formula (3), an end group represented by Formula (3-1) or (3-2) is more preferable.

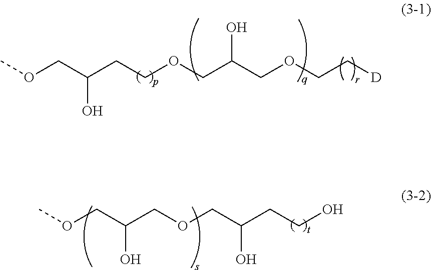

In Formula (3-1), p represents an integer of 0 to 3, q represents an integer of 0 to 2, and r represents an integer of 0 to 5. The total value of p and r is in a range of 1 to 5. D represents a polar group, a vinyl group, an ethynyl group, or an aryl group which may have a substituent.

(In Formula (3-2), s represents an integer of 0 to 2. t represents an integer of 1 to 5.)

Each polar group contained in the end group represented by Formulae (3-1) and (3-2) is bonded to different carbon atoms. In Formulae (3-1) and (3-2), carbon atoms to which the polar groups are bonded are bonded to each other through a linking group having a carbon atom to which the polar group is not bonded. Therefore, in the fluorine-containing ether compound represented by Formula (1) in which R¹ or R⁴ represents an end group represented by Formula (3-1) or (3-2), both the end polar group in Formula (3-1) or (3-2) and the hydroxy group adjacent to the end polar group can be aligned to be in close contact with the protective layer by the linking group having a carbon atom that is not bonded to the polar group in Formula (3-1) or (3-2). Therefore, a strong interaction with the protective layer is obtained.

In a case where R¹ or R⁴ represents an end group represented by Formula (3-1) or Formula (3-2), the polar groups in the end group are unlikely to aggregate, and the interaction with the protective layer is likely to occur. Therefore, in a case where R¹ or R⁴ represents an end group represented by Formula (3-1) or Formula (3-2), the end portion in the fluorine-containing ether compound is unlikely to float, and the adhesion to the protective layer is unlikely to be degraded as compared with a case where the end group in which carbon atoms to which the polar group is bonded are directly bonded to each other is disposed.

In the end group represented by Formula (3-1), the linking group between a carbon atom to which D selected from a polar group, a vinyl group, an ethynyl group, or an aryl group which may have a substituent is bonded, which is disposed at the end, and a carbon atom to which a hydroxy group adjacent to D, which is disposed at the end, is bonded has oxygen atom forming an ether bond. In Formula (3-1), p represents an integer of 0 to 3, q represents an integer of 0 to 2, r represents an integer of 0 to 5, and the total value of p and r is in a range of 1 to 5. Therefore, the above-described linking group has a linear structure formed of 2 to 7 atoms having a carbon atom that is not bonded to the polar group.

In the end group represented by Formula (3-1), since the above-described linking group has an oxygen atom forming an ether bond and has a linear structure formed of two or more atoms having a carbon atom that is not bonded to a polar group, the distance between D and the hydroxy group adjacent to D is appropriate. Therefore, the interaction between D and the hydroxy group adjacent to D in the molecules can be suppressed, and the polar group in the end group represented by Formula (3-1) can efficiently adhere to the protective layer. In addition, since the above-described linking group has a linear structure formed of two or more atoms, even in a case where the linking group has an oxygen atom forming an ether bond, a fluorine-containing ether compound having satisfactory hydrophobicity is obtained. In addition, since the above-described linking group has a linear structure formed of two or more atoms, even in a case where the linking group has an oxygen atom forming an ether bond, the molecular mobility is appropriate, the intramolecular aggregation is unlikely to occur, and the adhesion to the protective layer is excellent.

In the end group represented by Formula (3-1), since the above-described linking group has a linear structure formed of 7 or less atoms having an oxygen atom that forms an ether bond, and a carbon atom that is not bonded to a polar group, the hydrophobicity of the linking group is not extremely high so that the adhesion of the lubricating layer containing a fluorine-containing ether compound to the protective layer is not impaired.

Therefore, the lubricating layer containing the fluorine-containing ether compound having an end group represented by Formula (3-1), in which the linking group has a linear structure formed of 2 to 7 atoms having a carbon atom that is not bonded to a polar group, has excellent adhesion to the protective layer, exhibits high corrosion resistance, and has a high pickup suppression effect.

In Formula (3-1), the total value of p and r is in a range of 1 to 5 and preferably in a range of 1 to 3. In Formula (3-1), the carbon atom in the linking group disposed between the carbon atoms to which the polar groups are bonded prevents the intramolecular interaction between the adjacent polar groups from occurring preferentially to the interaction between the polar group and the protective layer, and improves the adhesion between the polar group in Formula (3-1) and the protective layer. Meanwhile, in a case where the number of carbon atoms in the above-described linking group is extremely large, the flexibility of the end group represented by Formula (3-1) is degraded, and it may be difficult to uniformly coat the protective layer. In the end group represented by Formula (3-1), since the total value of p and r is 5 or less, the alkylene chain of the main chain portion of Formula (3-1) is not extremely long. Therefore, since the rigid alkylene chain is long, it is possible to prevent degradation of the flexibility of the end portion so that the end portion can be prevented from floating due to a weak interaction with the protective layer. p represents preferably 0 or 1 and more preferably 0. r represents preferably 1 or 2 and more preferably 1.

D in Formula (3-1) represents a polar group, a vinyl group, an ethynyl group, or an aryl group which may have a substituent. In a case where D represents a polar group, it is preferable that D represents any of the polar groups described as the preferred examples of the polar group contained in the end group that does not correspond to Formula (2). Among the above-described polar groups, a polar group selected from the group consisting of a hydroxy group, a cyano group, or a group having an amide bond is more preferable. In a case where D represents an aryl group which may have a substituent, an aryl group which may have a substituent contained in the organic group which can be used in a case where B in Formula (3) described above represents an organic group having one or more carbon-carbon unsaturated bonds can be used.

In Formula (3-1), q represents an integer of 0 to 2. The number of polar groups in Formula (3-1) is q+2 in a case where D represents a polar group, and the number thereof is q+1 in a case where D represents a vinyl group or an ethynyl group. As described above, the number of polar groups included in R¹ and R⁴ is preferably 2 or more and 3 or less. Therefore, q in Formula (3-1) is preferably 0 or 1 in a case where D represents a polar group, and is preferably 1 or 2 in a case where D represents a vinyl group or an ethynyl group. In a case where D represents an aryl group which may have a substituent, it is preferable to select q so that the number of polar groups in Formula (3-1) is 2 or 3.

In the end group represented by Formula (3-2), a linking group between a carbon atom to which the end hydroxy group is bonded and a carbon atom to which a hydroxy group adjacent to the end hydroxy group is bonded does not have an oxygen atom. In this manner, since the intramolecular interaction is small and the intramolecular aggregation is unlikely to occur, the lubricating layer has excellent adhesion to the protective layer. t in Formula (3-2) represents an integer of 1 to 5. Therefore, the above-described linking group has a linear structure formed of 1 to 5 atoms having a carbon atom to which a hydroxy group is not bonded.

Since the above-described linking group in Formula (3-2) has a linear structure formed of one or more atoms having a carbon atoms to which a hydroxy group is not bonded, the distance between the end hydroxy group and the hydroxy group adjacent to the end hydroxy group is appropriate, intramolecular aggregation is unlikely to occur, and the hydrophobicity is satisfactory.

In addition, since the above-described linking group in Formula (3-2) does not have an oxygen atom forming an ether bond and has a linear structure formed of 5 or less atoms having a carbon atom to which a hydroxy group is not bonded, the hydrophobicity of the linking group is not extremely high so that the adhesion to the protective layer is not impaired, and the linking group is not extremely bulky so that the influence on the hindrance of movement of the hydroxy group is small.

In addition, in Formula (3-2), in a case where the number of carbon atoms in the linking group disposed between the carbon atoms to which the polar groups are bonded is extremely large, the flexibility of the end group represented by Formula (3-2) is degraded, and thus it may be difficult to uniformly coat the entire surface of the protective layer. In the end group represented by Formula (3-2), since t represents 5 or less, the alkylene chain in the main chain portion of Formula (3-2) is not extremely long. Therefore, since the rigid alkylene chain is long, the flexibility of the end portion is not degraded so that it is possible to prevent the interaction between the end hydroxy group and the protective layer from being degraded.

Accordingly, the lubricating layer containing the fluorine-containing ether compound in which the linking group has a linear structure formed of 1 to 5 atoms having a carbon atom to which a hydroxy group is not bonded without having an oxygen atom forming an ether bond has excellent adhesion to the protective layer, exhibits high corrosion resistance, and has a high pickup suppression effect.

t in Formula (3-2) represents preferably 1 or 2 and more preferably 1.

In Formula (3-2), s represents an integer of 0 to 2. The number of polar groups in Formula (3-2) is s+2, and the number of polar groups in R¹ and R⁴ is preferably 2 or more and 3 or less respectively as described above. Therefore, it is preferable that s in Formula (3-2) represents 0 or 1.

(Divalent linking group represented by R³) In the fluorine-containing ether compound represented by Formula (1), x pieces of R³'s represent a divalent linking group having one to four polar groups. R³'s are disposed between adjacent PFPE chains through a methylene group. In this manner, R³'s cause the fluorine-containing ether compound to adhere to the protective layer. As a result, the lubricant containing the fluorine-containing ether compound according to the present embodiment is capable of forming a thin lubricating layer with a sufficient coating rate.

In Formula (1), in a case where x represents 2, two R³'s may be the same as or different from each other, but it is preferable that two R³'s are the same as each other. In a case where two R³'s are the same as each other, the state of coating the protective layer with the fluorine-containing ether compound is more uniform, and a lubricating layer having more satisfactory adhesion can be formed. In addition, in a case where two R³'s are the same as each other, the fluorine-containing ether compound can be easily and efficiently produced.

In the present specification, in a case where x represents 2, the expression “two R³'s are the same as each other” denotes that the atoms in the two R³'s are symmetrically disposed with respect to R² disposed at the center of the chain structure of the molecules.

x pieces of R³'s in Formula (1) each have one or more polar groups. Therefore, in a case where a lubricating layer is formed on the protective layer using a lubricant containing a fluorine-containing ether compound, a suitable interaction occurs between the lubricating layer and the protective layer. Accordingly, the fluorine-containing ether compound represented by Formula (1) has excellent adhesion to the protective layer, and a lubricating layer having a high pickup suppression effect can be formed.

In addition, since the number of polar groups in R³ is 4 or less, in the lubricating layer containing the fluorine-containing ether compound, the polarity of the fluorine-containing ether compound is not extremely high, and aggregation of the fluorine-containing ether compound and formation of a lump are suppressed so that the lubricating layer can be suppressed from losing the smoothness. Therefore, the occurrence of the pickup due to the collision between the lubricating layer and the magnetic head can be suppressed. In addition, in a case where the number of polar groups in R³ is 4 or less, the hydrophilicity of the fluorine-containing ether compound is not extremely high, and water, which causes corrosion, can be prevented from being taken into the magnetic recording medium. As a result, a fluorine-containing ether compound capable of forming a lubricating layer having high corrosion resistance is obtained. The number of polar groups in R³ is preferably 3 or less.

It is preferable that the polar group in R³ is at least one polar group selected from the group consisting of a hydroxy group (—OH), a carboxy group (—COOH), a formyl group (—(C═O)H), a carbonyl group (—(C═O)R⁷; R⁷ represents an organic group), a sulfo group (—SO₃H), a cyano group (—CN), a group having an amide bond (—NR⁸COR⁹ or —CONR¹⁰R¹¹; R⁸, R⁹, R¹⁰, and R¹¹ each independently represent a hydrogen atom or an organic group), and an amino group (—NR¹²R¹³; R¹² and R¹³ each independently represent a hydrogen atom or an organic group). The group having an amide bond includes, as shown in the formula above, both a group (for example, a carboxamide group (—C(═O)NH₂)) bonded to a carbon atom constituting the amide bond and a group (for example, an acetamido group (—NHC(═O)CH₃)) bonded to a nitrogen atom constituting the amide bond. In the group having an amide bond, R⁸ and R⁹ may be bonded to each other to form a ring, and R¹⁰ and R¹¹ may be bonded to each other to form a ring. It is preferable that R¹, R⁹, R¹⁰, and R¹ in the group having an amide bond are each independently selected from the group consisting of a hydrogen atom, a methyl group, an ethyl group, a propyl group, and a butyl group.

It is preferable that R³'s each independently contain at least one polar group selected from the group consisting of a hydroxy group, a cyano group, or a group having an amide bond. The reason for this is that the hydroxy group, the cyano group, and the group having an amide bond are chemically stable, and a lubricating layer containing a fluorine-containing ether compound having these polar groups does not deteriorate for a long period of time. In addition, it is because the acidity of the hydroxy group, the cyano group, and the group having an amide bond is not extremely high, and thus the substrate is unlikely to be corroded.

In Formula (1), one to four polar groups in each of x pieces of R³'s may be partially or entirely the same as or different from each other.

It is preferable that x pieces of R³'s in Formula (1) each contain at least one hydroxy group and more preferable that all polar groups in x pieces of R³'s are hydroxy groups. This is because the state of coating the protective layer with the fluorine-containing ether compound is more uniform.

x pieces of R³'s in Formula (1) each represent preferably a linking group having 1 to 50 carbon atoms, more preferably a linking group having 3 to 50 carbon atoms, still more preferably a linking group having 3 to 20 carbon atoms, and most preferably a linking group having 4 to 15 carbon atoms. In a case where the number of carbon atoms in the linking group represented by R³ is 1 or more, since the hydrophobicity of the linking group can be ensured, water, which causes corrosion, can be prevented from being attracted to the lubricating layer, and thus a lubricating layer having satisfactory corrosion resistance can be formed. In a case where the number of carbon atoms in the linking group represented by R³ is 50 or less, the linking group has a flexible structure, and thus adhesion between the lubricating layer containing the fluorine-containing ether compound and the protective layer is improved. As a result, a lubricating layer capable of suppressing the pickup is obtained.

It is preferable that x pieces of R³'s in Formula (1) each represent a divalent linking group in which oxygen atoms are disposed at both ends. In this case, the oxygen atoms disposed at both ends of the divalent linking group represented by R³ form a methylene group (—CH₂—) disposed on both sides of R³ and an ether bond (—O—). These ether bonds impart moderate flexibility to the fluorine-containing ether compound represented by Formula (1), and increase the affinity between the polar group of the divalent linking group represented by R³ and the protective layer.

It is more preferable that x pieces of R³'s in Formula (1) each independently represent a divalent linking group having 3 to 50 carbon atoms, which has 1 to 3 hydroxy groups and oxygen atoms at both ends which are bonded to adjacent methylene groups.

More specifically, it is preferable that x pieces of R³'s each independently represent any one selected from the linking groups represented by Formulae (4-1) to (4-6).

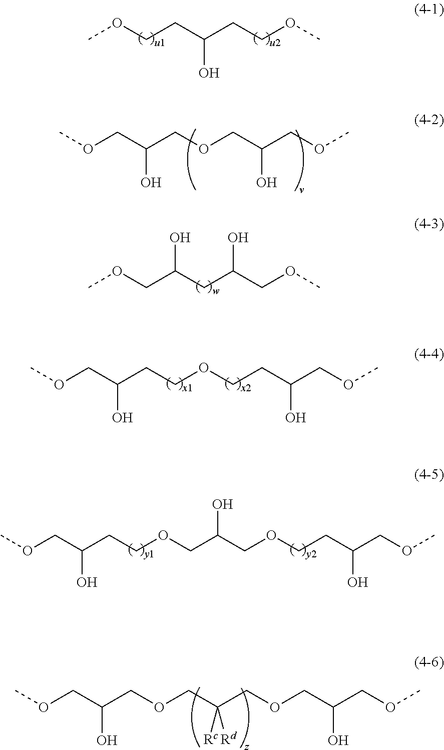

(In Formula (4-1), u1 represents an integer of 0 to 6, and u2 represents an integer of 0 to 6. Here, at least one of u1 or u2 represents 0. The oxygen atom at the left end of Formula (4-1) is bonded to the methylene group on the R¹ side in Formula (1), and the oxygen atom at the right end is bonded to the methylene group on the R⁴ side in Formula (1).)

(In Formula (4-2), v represents an integer of 1 or 2. The oxygen atom at the left end of Formula (4-2) is bonded to the methylene group on the R¹ side in Formula (1), and the oxygen atom at the right end is bonded to the methylene group on the R⁴ side in Formula (1).)

(In Formula (4-3), w represents an integer of 0 to 6. The oxygen atom at the left end of Formula (4-3) is bonded to the methylene group on the R¹ side in Formula (1), and the oxygen atom at the right end is bonded to the methylene group on the R⁴ side in Formula (1).)

(In Formula (4-4), x1 represents an integer of 0 to 5, and x2 represents an integer of 0 to 5. Here, at least one of x1 or x2 represents an integer of 1 to 5. The oxygen atom at the left end of Formula (4-4) is bonded to the methylene group on the R¹ side in Formula (1), and the oxygen atom at the right end is bonded to the methylene group on the R⁴ side in Formula (1).)

(In Formula (4-5), y1 represents an integer of 1 to 5, and y2 represents an integer of 1 to 5. The oxygen atom at the left end of Formula (4-5) is bonded to the methylene group on the R¹ side in Formula (1), and the oxygen atom at the right end is bonded to the methylene group on the R⁴ side in Formula (1).)

(In Formula (4-6), z represents an integer of 1 to 6. z pieces of R^{c}'s and R^{d}'s each independently represent a hydrogen atom, a fluorine atom, or a methyl group. The oxygen atom at the left end of Formula (4-6) is bonded to the methylene group on the R¹ side in Formula (1), and the oxygen atom at the right end is bonded to the methylene group on the R⁴ side in Formula (1).)

The linking group represented by Formula (4-1) has a glycerin structure (—OCH₂CH(OH)CH₂O—) or a structure in which 1 to 6 methylene groups are added to the glycerin structure. Therefore, the hydroxy group contained in the linking group represented by Formula (4-1) is only one, and the polarity of the linking group is maintained to be low. As a result, it is possible to effectively inhibit entrance of water, which causes the corrosion of the magnetic recording medium, and thus a lubricating layer having a high corrosion suppression effect on the magnetic recording medium can be formed.

In Formula (4-1), since at least one of u1 or u2 represents 0, the fluorine-containing ether compound represented by Formula (1) has excellent flexibility, and the state of coating the protective layer with the fluorine-containing ether compound is more uniform. In a case where only one of u1 or u2 represents 0, the value of the other which does not represent 0 in u1 and u2 is in a range of 1 to 6, preferably in a range of 1 to 4, and more preferably in a range of 1 to 3. This is because the linking group is not extremely rigid so that a lubricating layer having more satisfactory adhesion and excellent pickup resistance can be formed.

Further, in Formula (4-1), in a case where only one of u1 or u2 represents 0, the linking group has u1 or u2 pieces of carbon atoms that are not bonded to any of the polar group and the ether oxygen atom between the carbon atom bonded to a secondary hydroxy group and the ether oxygen atom forming an ether bond disposed at both ends. Therefore, the fluorine-containing ether compound that has excellent hydrophobicity due to u1 or u2 pieces of methylene groups and can form a lubricating layer having more satisfactory corrosion resistance is obtained.

The linking group represented by Formula (4-2) has a structure in which two or three glycerin structures (—OCH₂CH(OH)CH₂O—) are linked to each other. Since the glycerin structure imparts flexibility to the linking group, the linking group represented by Formula (4-2) is extremely flexible. As a result, the fluorine-containing ether compound represented by Formula (1) has more satisfactory adhesion, and a lubricating layer having more excellent pickup resistance can be formed.

In Formula (4-2), v represents an integer of 1 or 2 and preferably 1. In a case where v represents 2 or less, the polarity of the fluorine-containing ether compound represented by Formula (1) is maintained to be low, and thus a lubricating layer having more excellent corrosion resistance can be formed.

The linking group represented by Formula (4-3) has a structure in which carbon atoms to which hydroxy groups are bonded are directly bonded to each other, or a structure in which the carbon atoms are bonded through an alkylene group having 1 to 6 carbon atoms, which does not have an ether bond. Therefore, the linking group represented by Formula (4-3) has polarity maintained to be low as compared with, for example, a linking group having a structure in which carbon atoms, to which hydroxy groups are bonded, are bonded to each other through an oxygen atom forming an ether bond and an alkylene group. As a result, it is possible to effectively inhibit entrance of water, which causes the corrosion of the magnetic recording medium, and thus a lubricating layer having a high corrosion suppression effect on the magnetic recording medium can be formed.

In Formula (4-3), w represents an integer of 0 to 6 and preferably 0 to 4. In a case where w represents 6 or less, the linking group represented by Formula (4-3) is not extremely rigid so that a fluorine-containing ether compound capable of forming a lubricating layer having more satisfactory adhesion and excellent pickup resistance is obtained.

In addition, in a case where w in Formula (4-3) represents 1 or greater, the linking group has w pieces of carbon atoms that are not bonded to any of the polar group and the ether oxygen atom between the two carbon atoms to which secondary hydroxy groups are bonded. Therefore, the fluorine-containing ether compound has excellent hydrophobicity due to w pieces of methylene groups, and can form a lubricating layer having more satisfactory corrosion resistance. In a case where w represents 1 or greater, it is preferable that w represents 2 to 4.

The linking group represented by Formula (4-4) has a structure in which carbon atoms, to which hydroxy groups are bonded, are bonded to each other through a linear linking chain having 4 or more atoms having an oxygen atom forming an ether bond. In the fluorine-containing ether compound having a linking group represented by Formula (4-4), since the distance between two hydroxy groups in Formula (4-4) is sufficiently maintained, the intramolecular interaction of the hydroxy groups is suppressed, and thus a lubricating layer having satisfactory adsorption ability to the protective layer can be formed. Therefore, the lubricating layer containing the fluorine-containing ether compound represented by Formula (1) has more satisfactory adhesion and more excellent pickup resistance.

In Formula (4-4), x1 and x2 each independently represent an integer of 0 to 5, and at least one of x1 or x2 represents an integer of 1 to 5. Since the total value of x1 and x2 in the linking group represented by Formula (4-4) is 1 or greater, the hydrophobicity of the fluorine-containing ether compound is enhanced, and the lubricating layer containing the fluorine-containing ether compound has satisfactory corrosion resistance because water, which causes corrosion, is unlikely to be taken into the layer. It is preferable that x1 and x2 each independently represent an integer of 3 or less. This is because the linking group is not extremely rigid so that a lubricating layer having more satisfactory adhesion and excellent pickup resistance can be formed. In addition, the total value of x1 and x2 is preferably 4 or less. In order to impart moderate flexibility to the linking group represented by Formula (4-4), it is preferable that one of x1 or x2 represents 0.

The linking group represented by Formula (4-5) has a structure which has three hydroxy groups and in which carbon atoms, to which adjacent hydroxy groups are bonded, are bonded to each other through a linear linking chain having 4 or more atoms having an oxygen atom forming an ether bond.

In the fluorine-containing ether compound having a linking group represented by Formula (4-5), the linking group represented by Formula (4-5) has three hydroxy groups so that the interaction between the hydroxy group and the protective layer is likely to be formed, and a lubricating layer having satisfactory adhesion to the protective layer can be formed. Further, since the fluorine-containing ether compound having the linking group represented by Formula (4-5) is formed such that the distance between adjacent hydroxy groups in the three hydroxy groups in Formula (4-5) is sufficiently maintained, the intramolecular interaction of the hydroxy groups is suppressed, and thus a lubricating layer having a satisfactory adsorption ability to the protective layer can be formed. Therefore, the lubricating layer containing the fluorine-containing ether compound represented by Formula (1) has more satisfactory adhesion and more excellent pickup resistance.

In Formula (4-5), y1 and y2 each independently represent an integer of 1 to 5. Since both of y1 and y2 in the linking group represented by Formula (4-5) represent 1 or greater, the hydrophobicity of the fluorine-containing ether compound is enhanced, and the lubricating layer containing the fluorine-containing ether compound has satisfactory corrosion resistance because water, which causes corrosion, is unlikely to be taken into the layer. In order to impart moderate flexibility to the linking group represented by Formula (4-5), it is preferable that y1 and y2 each independently represent 1 to 3 and most preferable that both y1 and y2 represent 1. In addition, since the state of coating the protective layer with the fluorine-containing ether compound is more uniform, it is preferable that y1 and y2 are the same as each other.

The linking group represented by Formula (4-6) has a structure in which carbon atoms, to which hydroxy groups are bonded, are bonded to each other through a linear linking chain having 7 or more atoms having two ether bonds. In the linking group represented by Formula (4-6), since the distance between the two hydroxy groups is sufficiently maintained, the intramolecular interaction is suppressed. Therefore, the linking group represented by Formula (4-6) has a satisfactory adsorption ability to the protective layer. As a result, the fluorine-containing ether compound having a linking group represented by Formula (4-6) has more satisfactory adhesion and is capable of forming a lubricating layer having excellent pickup resistance.

In Formula (4-6), z represents an integer of 1 to 6. In order to impart moderate flexibility to the linking group represented by Formula (4-6), it is preferable that z represents 4 or less. In addition, in order to impart sufficient hydrophobicity to the linking group represented by Formula (4-6), it is preferable that z represents 2 or greater.

z pieces of R's and R^{d}'s in Formula (4-6) each independently represent a hydrogen atom, a fluorine atom, or a methyl group. Since the production is facilitated, it is preferable that each of z pieces of (—CR^{c}R^{d}—)'s represents any of —CH₂—, —CH(CH₃)—, —C(CH₃)₂—, or —CF₂—.

A case where z pieces of (—CR^{c}R^{d}—)'s are only one or a plurality of —CH₂-'s is preferable because, for example, the linking group represented by Formula (4-6) is flexible as compared to a case of including —CH(CH₃)— and/or —C(CH₃)₂—.

In addition, a case where at least some of the z pieces of (—CR^{c}R^{d}—)'s include —CH(CH₃)— and/or —C(CH₃)₂— is preferable because the linking group is moderately bulky and the intramolecular interaction between the hydroxy groups of the linking group represented by Formula (4-6) can be effectively suppressed as compared with a case where all the z pieces of (—CR^{c}R^{d}—)'s are —CH₂—.

In addition, a case where z pieces of (—CR^{c}R^{d}—)'s include one or a plurality of —CF₂-'s is preferable because the affinity for water of the linking group represented by Formula (4-6) is lowered, and it is more difficult to take in water, which causes corrosion.

### (PFPE Chain Represented by R²)

In the fluorine-containing ether compound represented by Formula (1), (x+1) pieces of R²'s each independently represent a perfluoropolyether chain (hereinafter, referred to as “PFPE chain”). In a case where the lubricant containing the fluorine-containing ether compound according to the present embodiment is applied onto the protective layer to form a lubricating layer, the surface of the protective layer is coated with the PFPE chain represented by R², lubricity is imparted to the lubricating layer, and a frictional force between the magnetic head and the protective layer is reduced. The PFPE chain represented by R² is appropriately selected according to the performance required for the lubricant containing the fluorine-containing ether compound.

In the fluorine-containing ether compound represented by Formula (1), (x+1) pieces of R²'s may be partially or entirely the same as or different from each other. It is preferable that (x+1) pieces of R²'s are all the same as each other. This is because the state coating the protective layer with the fluorine-containing ether compound is uniform, and a lubricating layer having more satisfactory adhesion is formed. In a case where two or more of R²'s among (x+1) pieces of R²'s are the same as each other, this denotes that (x+1) pieces of R²'s include two or more R²'s having the same structure of the repeating unit of the PFPE chain. R²'s having the same structure described above include R²'s having the same structure of the repeating unit and different average degree of polymerizations.

Examples of the PFPE chain represented by R² include a polymer or a copolymer of perfluoroalkylene oxide. Examples of the perfluoroalkylene oxide include perfluoromethylene oxide, perfluoroethylene oxide, perfluoro-n-propylene oxide, perfluoroisopropylene oxide, and perfluorobutylene oxide.

It is preferable that (x+1) pieces of R²'s in Formula (1) each independently represent a PFPE chain represented by Formula (5) derived from, for example, a polymer or a copolymer of perfluoroalkylene oxide.

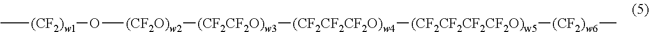

(In Formula (5), each w2, w3, w4, and w5 represents an average degree of polymerization, and each independently represent 0 to 20. Here, all of w2, w3, w4, and w5 do not represent 0 at the same time. Each w1 and w6 represents an average value representing the number of CF₂'s, and each independently represent 1 to 3. The arrangement order of (CF₂O), (CF₂CF₂O), (CF₂CF₂CF₂O), and (CF₂CF₂CF₂CF₂O), which are the repeating units in Formula (5), is not particularly limited.)

In Formula (5), each w2, w3, w4, and w5 represents an average degree of polymerization, and each independently represent 0 to 20, preferably 0 to 15, and more preferably 0 to 10.

In Formula (5), each w1 and w6 represents an average value indicating the number of CF₂'s, and each independently represent 1 to 3. w1 and w6 are determined depending on the structure of the repeating unit disposed at the end of the chain structure in the PFPE chain represented by Formula (5).

(CF₂O), (CF₂CF₂O), (CF₂CF₂CF₂O), and (CF₂CF₂CF₂CF₂O) in Formula (5) are repeating units. The arrangement order of the repeating units in Formula (5) is not particularly limited. In addition, the number of kinds of repeating units in Formula (5) is also not particularly limited.

It is preferable that (x+1) pieces of R²'s in Formula (1) each independently represent any one selected from the PFPE chains represented by Formulae (5-1) to (5-4).

In a case where (x+1) pieces of R²'s each independently represent any one selected from the PFPE chains represented by Formulae (5-1) to (5-4), a fluorine-containing ether compound capable of obtaining a lubricating layer having satisfactory lubricity is obtained. In addition, in a case where (x+1) pieces of R²'s each independently represent any one selected from the PFPE chains represented by Formulae (5-1) to (5-4), the ratio of the number of oxygen atoms (number of ether bonds (—O—)) to the number of carbon atoms in the PFPE chain is appropriate. Therefore, the fluorine-containing ether compound has moderate hardness. Therefore, the fluorine-containing ether compound applied onto the protective layer is unlikely to be aggregated on the protective layer, and a lubricating layer having a thinner thickness can be formed with a sufficient coating rate. In addition, the lubricating layer containing the fluorine-containing ether compound in which (x+1) pieces of R²'s each independently represent any one selected from the PFPE chains represented by Formulae (5-1) to (5-4) is denser, and the pickup can be further suppressed, which is preferable.

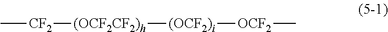

(In Formula (5-1), each h and i represents an average degree of polymerization, where h represents 1 to 20 and i represents 0 to 20.)

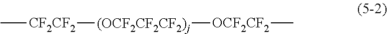

(In Formula (5-2), j represents an average degree of polymerization, and represents 1 to 15.)

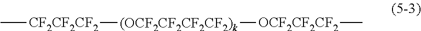

(In Formula (5-3), k represents an average degree of polymerization, and represents 1 to 10.)

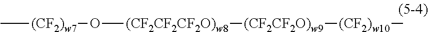

(In Formula (5-4), each w8 and w9 represents an average degree of polymerization, and each independently represents 1 to 20. Each w7 and w10 represents an average value representing the number of CF₂'s, and each independently represent 1 to 2.)

In Formula (5-1), the arrangement order of (OCF₂CF₂) and (OCF₂) which are the repeating units is not particularly limited. In Formula (5-1), the number h of (OCF₂CF₂)'s and the number i of (OCF₂)'s may be the same as or different from each other. The PFPE chain represented by Formula (5-1) may be a polymer of (OCF₂CF₂). In addition, the PFPE chain represented by Formula (5-1) may be any of a random copolymer, a block copolymer, or an alternating copolymer consisting of (OCF₂CF₂) and (OCF₂).

In Formulae (5-1) to (5-3), since h indicating the average degree of polymerization represents 1 to 20, i represents 0 to 20, j represents 1 to 15, and k represents 1 to 10, a fluorine-containing ether compound capable of obtaining a lubricating layer having satisfactory lubricity is obtained. In addition, in Formulae (5-1) to (5-3), since h and i indicating the average degree of polymerization represent 20 or less, j represents 15 or less, and k represents 10 or less, the viscosity of the fluorine-containing ether compound is not extremely high, and a lubricant containing the fluorine-containing ether compound is easy to apply, which is preferable. h, i, j, and k indicating the average degree of polymerization represent preferably 1 to 10, more preferably 1.5 to 8, and still more preferably 2 to 7 from the viewpoint that the fluorine-containing ether compound that is easily wet and spreads on the protective layer and easily obtains a lubricating layer having a uniform film thickness is obtained.

In Formula (5-4), the arrangement order of (CF₂CF₂CF₂O) and (CF₂CF₂O), which are the repeating units, is not particularly limited. In Formula (5-4), the number w8 of (CF₂CF₂CF₂O)'s indicating the average degree of polymerization and the number w9 of (CF₂CF₂O)'s may be the same as or different from each other. Formula (5-4) may represent any of a random copolymer, a block copolymer, or an alternating copolymer, formed of monomer units (CF₂CF₂CF₂O) and (CF₂CF₂O).

In Formula (5-4), w8 and w9 indicating the average degree of polymerization each independently represent 1 to 20, preferably 1 to 15, and more preferably 1 to 10. w7 and w10 in Formula (5-4) are average values indicating the number of CF₂'s, and each independently represent 1 to 2. w7 and w10 are determined according to the structure of the repeating unit disposed at the end of the chain structure in the PFPE chain represented by Formula (5-4).

Specifically, the fluorine-containing ether compound represented by Formula (1) is preferably any of compounds represented by Formulae (AA) to (AT), (BA) to (BL), (CA) to (CF), and (DA) to (DH).

In a case where the fluorine-containing ether compound represented by Formula (1) is any of compounds represented by Formulae (AA) to (AT), (BA) to (BL), (CA) to (CF), or (DA) to (DH), a raw material is easily available, and a lubricating layer having more satisfactory corrosion resistance and a higher pickup suppression effect can be formed even in a case where the thickness is thin.

In the compounds represented by Formulae (AA) to (AT), (BA) to (BL), (CA) to (CF), and (DA) to (DH), Rf₁, Rf₂, and Rf₃ representing a PFPE chain each have the following structures. That is, in the compounds represented by Formulae (AA) to (AT), (BA) to (BJ), (CA) to (CF), (DA), and (DB), Rf₁ represents the PFPE chain represented by Formula (5-1). In the compounds represented by Formulae (BK) and (DC) to (DH), Rf₂ represents the PFPE chain represented by Formula (5-2). In the compound represented by Formula (BL), Rf₃ represents the PFPE chain represented by Formula (5-3). Further, in Formulae (AA) to (AT), (BA) to (BL), (CA) to (CF), and (DA) to (DH), h and i in Rf₁ representing the PFPE chain, j in Rf₂, and k in Rf₃ are values indicating the average degree of polymerization, and thus are not necessarily integers.

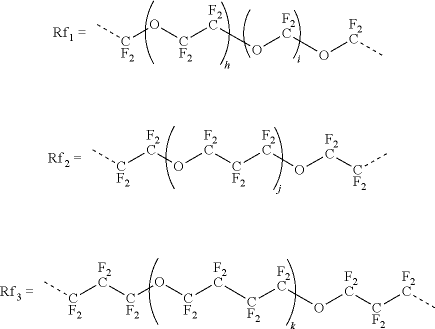

In the compounds represented by Formulae (AA) to (AN), x in Formula (1) represents 1. R¹ and R⁴ are the same as each other and represent an end group represented by any of Formulae (2-1) to (2-6). Two R²'s are the same as each other and represent the PFPE chain represented by Formula (5-1). R³ represents a linking group represented by Formula (4-1).

In the compounds represented by Formulae (AO) to (AT), x in Formula (1) represents 1. R¹ represents an end group represented by Formula (2-1), and R⁴ represents an end group represented by any of Formula (3-1), Formula (3-2), or Formula (3) which does not correspond to Formula (3-1) and Formula (3-2). Two R²'s are the same as each other and represent the PFPE chain represented by Formula (5-1). R³ represents a linking group represented by Formula (4-1).

In the compounds represented by Formulae (BA) to (BJ), x in Formula (1) represents 1. R¹ and R⁴ are the same as each other and represent an end group represented by Formula (2-1). Two R²'s are the same as each other and represent the PFPE chain represented by Formula (5-1). R³ represents a linking group represented by any of Formulae (4-1) to (4-6).

In the compounds represented by Formulae (BK) and (BL), x in Formula (1) represents 1. R¹ and R⁴ are the same as each other and represent an end group represented by Formula (2-1). Two R²'s are the same as each other and represent the PFPE chain represented by Formula (5-2) or (5-3). R³ represents a linking group represented by Formula (4-1).

In the compounds represented by Formulae (CA) to (CD), x in Formula (1) represents 2. R¹ and R⁴ are the same as each other and represent an end group represented by any of Formulae (2-1) to (2-4). Three R²'s are the same as each other and represent a PFPE chain represented by Formula (5-1). Two R³'s are the same as each other and represent a linking group represented by Formula (4-1).

In the compounds represented by Formulae (CE) to (CF), x in Formula (1) represents 2. R¹ and R⁴ are the same as each other and represent an end group represented by Formula (2-1). Three R²'s are the same as each other and represent a PFPE chain represented by Formula (5-1). Two R³'s are the same as each other and represent a linking group represented by Formula (4-2) or (4-3).

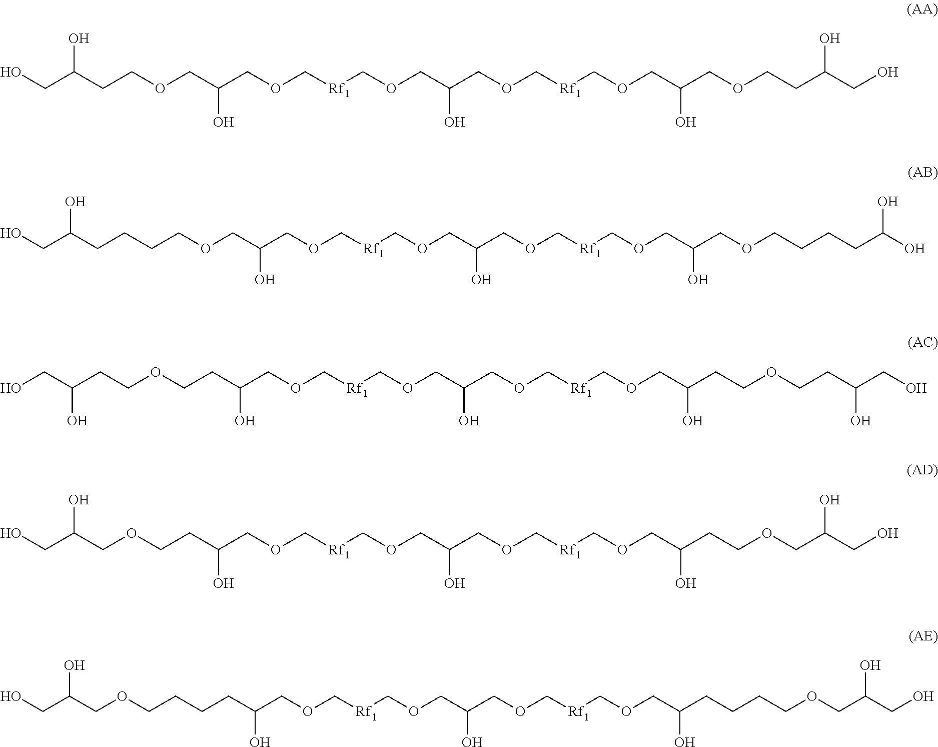

(In two Rf₁'s in Formula (AB), h and i represent an average degree of polymerization, h represents 1 to 20, and i represents 0 to 20. h and i in the two Rf₁'s may be the same as or different from each other.)

(In two Rf₁'s in Formula (AC), h and i represent an average degree of polymerization, h represents 1 to 20, and i represents 0 to 20. h and i in the two Rf₁'s may be the same as or different from each other.)

(In two Rf₁'s in Formula (AD), h and i represent an average degree of polymerization, h represents 1 to 20, and i represents 0 to 20. h and i in the two Rf₁'s may be the same as or different from each other.)

(In two Rf₁'s in Formula (AE), h and i represent an average degree of polymerization, h represents 1 to 20, and i represents 0 to 20. h and i in the two Rf₁'s may be the same as or different from each other.)

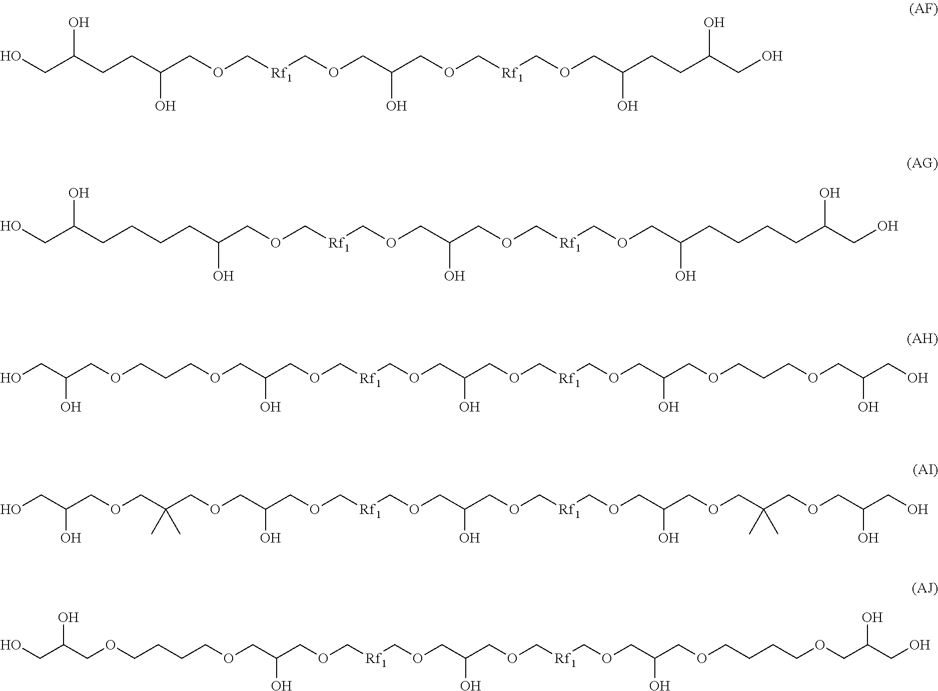

(In two Rf₁'s in Formula (AF), h and i represent an average degree of polymerization, h represents 1 to 20, and i represents 0 to 20. h and i in the two Rf₁'s may be the same as or different from each other.)

(In two Rf₁'s in Formula (AG), h and i represent an average degree of polymerization. h represents 1 to 20. and i represents 0 to 20. h and i in the two Rf₁'s may be the same as or different from each other.)

(In two Rf₁'s in Formula (AH), h and i represent an average degree of polymerization, h represents 1 to 20, and i represents 0 to 20. h and i in the two Rf₁'s may be the same as or different from each other.)

(In two Rf₁'s in Formula (AI), h and i represent an average degree of polymerization, h represents 1 to 20, and i represents 0 to 20. h and i in the two Rf₁'s may be the same as or different from each other.)

(In two Rf₁'s in Formula (AJ), h and i represent an average degree of polymerization, h represents 1 to 20, and i represents 0 to 20. h and i in the two Rf₁'s may be the same as or different from each other.)

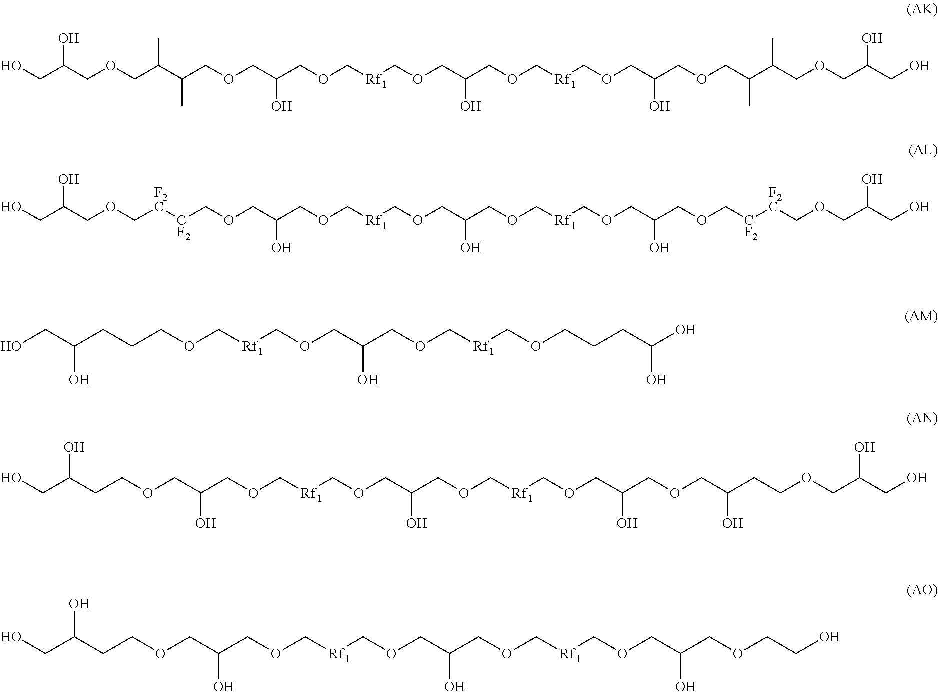

(In two Rf₁'s in Formula (AK), h and i represent an average degree of polymerization, h represents 1 to 20, and i represents 0 to 20. h and i in the two Rf₁'s may be the same as or different from each other.)

In two Rf₁'s in Formula (AL), h and i represent an average degree of polymerization, h represents 1 to 20, and i represents 0 to 20. h and i in the two Rf₁'s may be the same as or different from each other.)

(In two Rf₁'s in Formula (AM), h and i represent an average degree of polymerization, h represents 1 to 20, and i represents 0 to 20. h and i in the two Rf₁'s may be the same as or different from each other.)

(In two Rf₁'s in Formula (AN), h and i represent an average degree of polymerization, h represents 1 to 20, and i represents 0 to 20. h and i in the two Rf₁'s may be the same as or different from each other.)

(In two Rf₁'s in Formula (AO), h and i represent an average degree of polymerization, h represents 1 to 20, and i represents 0 to 20. h and i in the two Rf₁'s may be the same as or different from each other.)

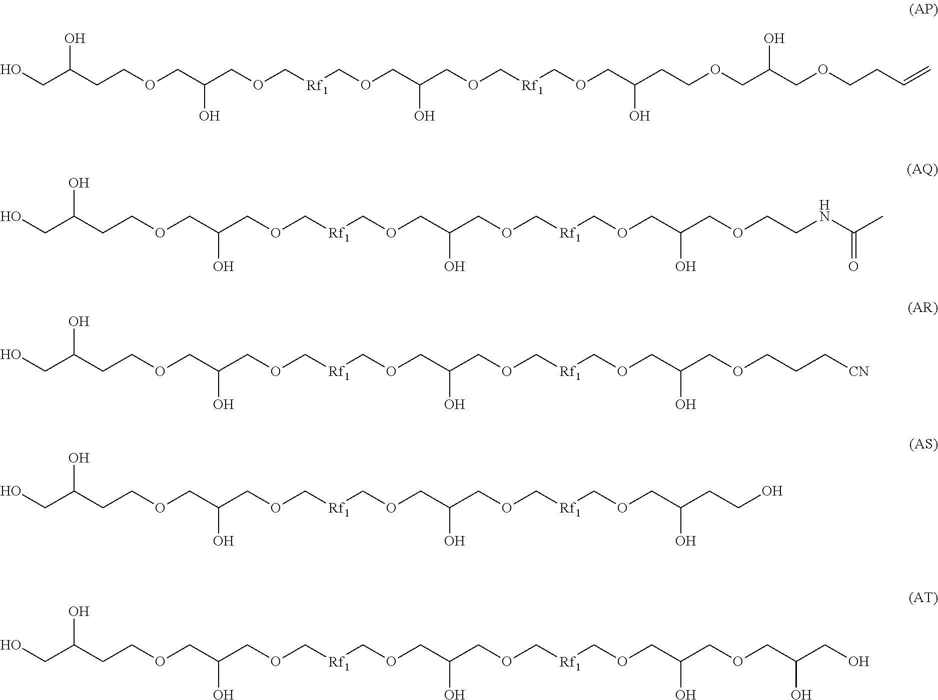

(In two Rf₁'s in Formula (AP), h and i represent an average degree of polymerization, h represents 1 to 20, and i represents 0 to 20. h and i in the two Rf₁'s may be the same as or different from each other.)

(In two Rf₁'s in Formula (AQ), h and i represent an average degree of polymerization, h represents 1 to 20, and i represents 0 to 20. h and i in the two Rf₁'s may be the same as or different from each other.)

(In two Rf₁'s in Formula (AR). h and i represent an average degree of polymerization, h represents 1 to 20, and i represents 0 to 20. h and i in the two Rf₁'s may be the same as or different from each other.)

(In two Rf₁'s in Formula (AS), h and i represent an average degree of polymerization, h represents 1 to 20, and i represents 0 to 20. h and i in the two Rf₁'s may be the same as or different from each other.)

(In two Rf₁'s in Formula (AT), h and i represent an average degree of polymerization, h represents 1 to 20, and i represents 0 to 20. h and i in the two Rf₁'s may be the same as or different from each other.)

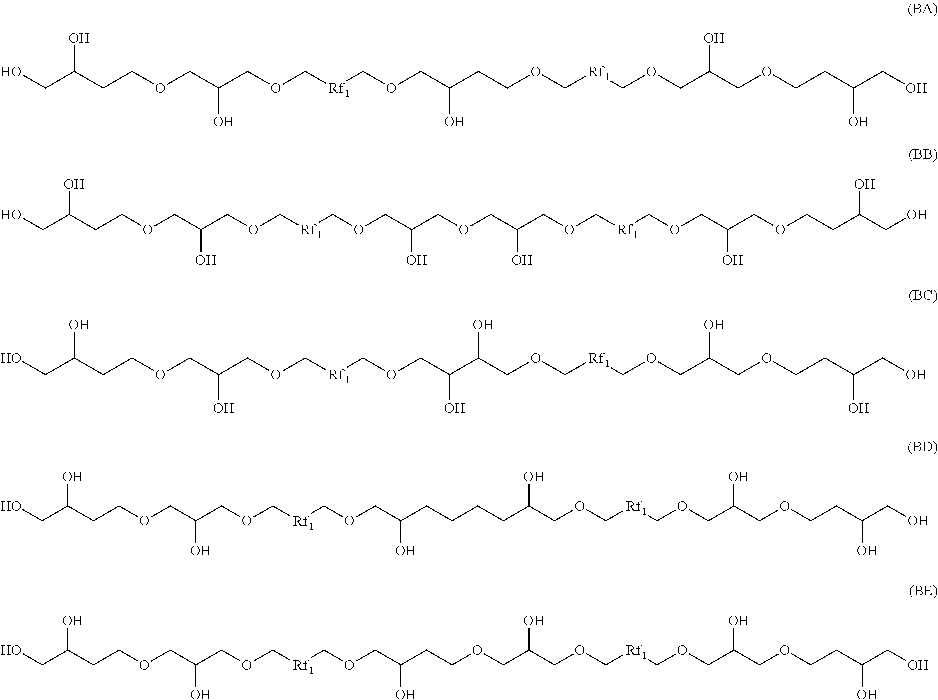

(In two Rf₁'s in Formula (BA), h and i represent an average degree of polymerization, h represents 1 to 20, and i represents 0 to 20. h and i in the two Rf₁'s may be the same as or different from each other.)

(In two Rf₁'s in Formula (BB), h and i represent an average degree of polymerization, h represents 1 to 20, and i represents 0 to 20. h and i in the two Rf₁'s may be the same as or different from each other.)

(In two Rf₁'s in Formula (BC), h and i represent an average degree of polymerization, h represents 1 to 20, and i represents 0 to 20. h and i in the two Rf₁'s may be the same as or different from each other.)

(In two Rf₁'s in Formula (BD), h and i represent an average degree of polymerization, h represents 1 to 20, and i represents 0 to 20. h and i in the two Rf₁'s may be the same as or different from each other.)

(In two Rf₁'s in Formula (BE), h and i represent an average degree of polymerization, h represents 1 to 20, and i represents 0 to 20. h and i in the two Rf₁'s may be the same as or different from each other.)

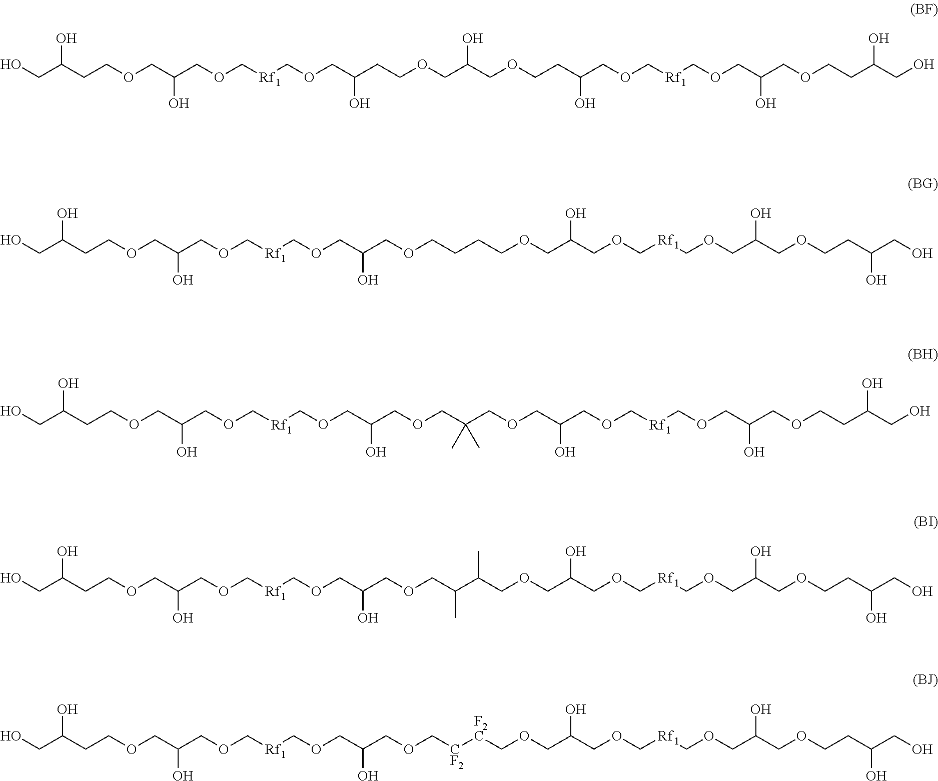

(In two Rf₁'s in Formula (BF), h and i represent an average degree of polymerization, h represents 1 to 20, and i represents 0 to 20. h and i in the two Rf₁'s may be the same as or different from each other.)

(In two Rf₁'s in Formula (BG), h and i represent an average degree of polymerization, h represents 1 to 20, and i represents 0 to 20. h and i in the two Rf₁'s may be the same as or different from each other.)

(In two Rf₁'s in Formula (BH), h and i represent an average degree of polymerization, h represents 1 to 20, and i represents 0 to 20. h and i in the two Rf₁'s may be the same as or different from each other.)

(In two Rf₁'s in Formula (BI), h and i represent an average degree of polymerization, h represents 1 to 20, and i represents 0 to 20. h and i in the two Rf₁'s may be the same as or different from each other.)

(In two Rf₁'s in Formula (BJ), h and i represent an average degree of polymerization, h represents 1 to 20, and i represents 0 to 20. h and i in the two Rf₁'s may be the same as or different from each other.)

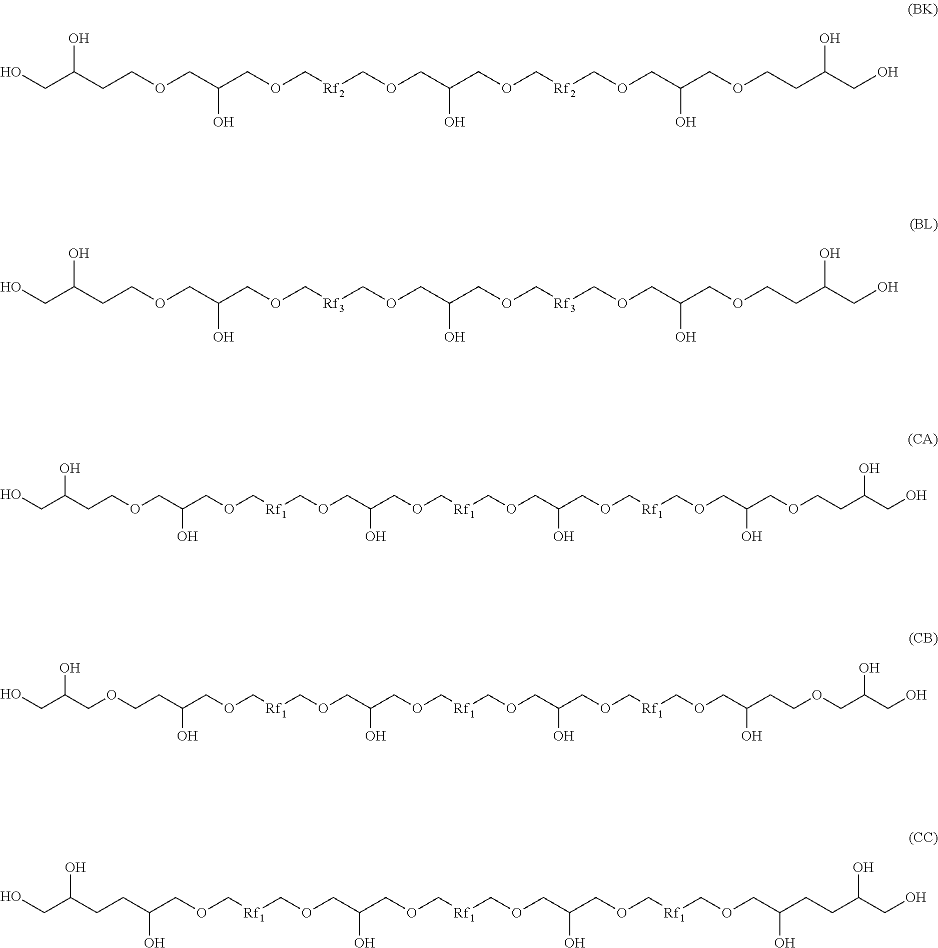

(In two Rf₂'s in Formula (BK), j represents an average degree of polymerization, and represents 1 to 15. j's in the two Rf₂'s may be the same as or different from each other.)

(In two Rf₃'s in Formula (BK), k represents an average degree of polymerization polymerization, and represents 1 to 10. k's in the two RU's may be the same as or different from each other.)

(In three Rf₁'s in Formula (CA), h and i represent an average degree of polymerization, h represents 1 to 20, and i represents 0 to 20. h's and i's in the three Rf₁'s may be partially or entirely the same as or different from each other.)

(In three Rf₁'s in Formula (CB), h and i represent an average degree of polymerization, h represents 1 to 20, and i represents 0 to 20. h's and i's in the three Rf₁'s may be partially or entirely the same as or different from each other.)

(In three Rf₁'s in Formula (CC), h and i represent an average degree of polymerization, h represents 1 to 20, and i represents 0 to 20. h's and i's in the three Rf₁'s may be partially or entirely the same as or different from each other.)

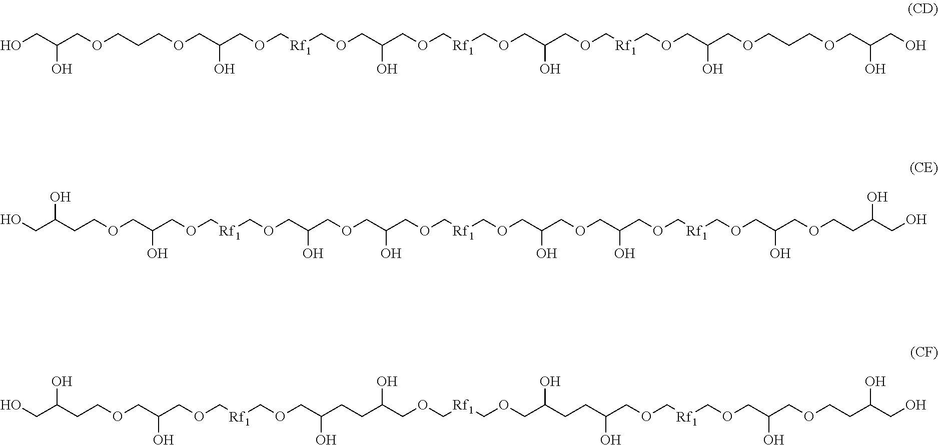

(In three Rf₁'s in Formula (CD), h and i represent an average degree of polymerization, h represents 1 to 20, and i represents 0 to 20. h's and i's in the three Rf₁'s may be partially or entirely the same as or different from each other.)

(In three Rf₁'s in Formula (CE), h and i represent an average degree of polymerization, h represents 1 to 20, and i represents 0 to 20. h's and i's in the three Rf₁'s may be partially or entirely the same as or different from each other.)

(In three Rf₁'s in Formula (CF), h and i represent an average degree of polymerization, h represents 1 to 20, and i represents 0 to 20. h's and i's in the three Rf₁'s may be partially or entirely the same as or different from each other.)

In the compounds represented by Formulae (DA) to (DG), x in Formula (1) represents 1. R¹ and R⁴ are the same as each other and represent an end group represented by any of Formulae (2-1), (2-2), (2-6), and (2-7). Two R²'s are the same as each other and represent a PFPE chain represented by Formula (5-1) or (5-2). R³ represents a linking group represented by Formula (4-1) or (4-4).

In the compound represented by Formula (DH), x in Formula (1) represents 2. R¹ and R⁴ are the same as each other and represent an end group represented by Formula (2-2). Three R²'s are the same as each other and represent a PFPE chain represented by Formula (5-2). Two R³'s are the same as each other and represent a linking group represented by Formula (4-1).

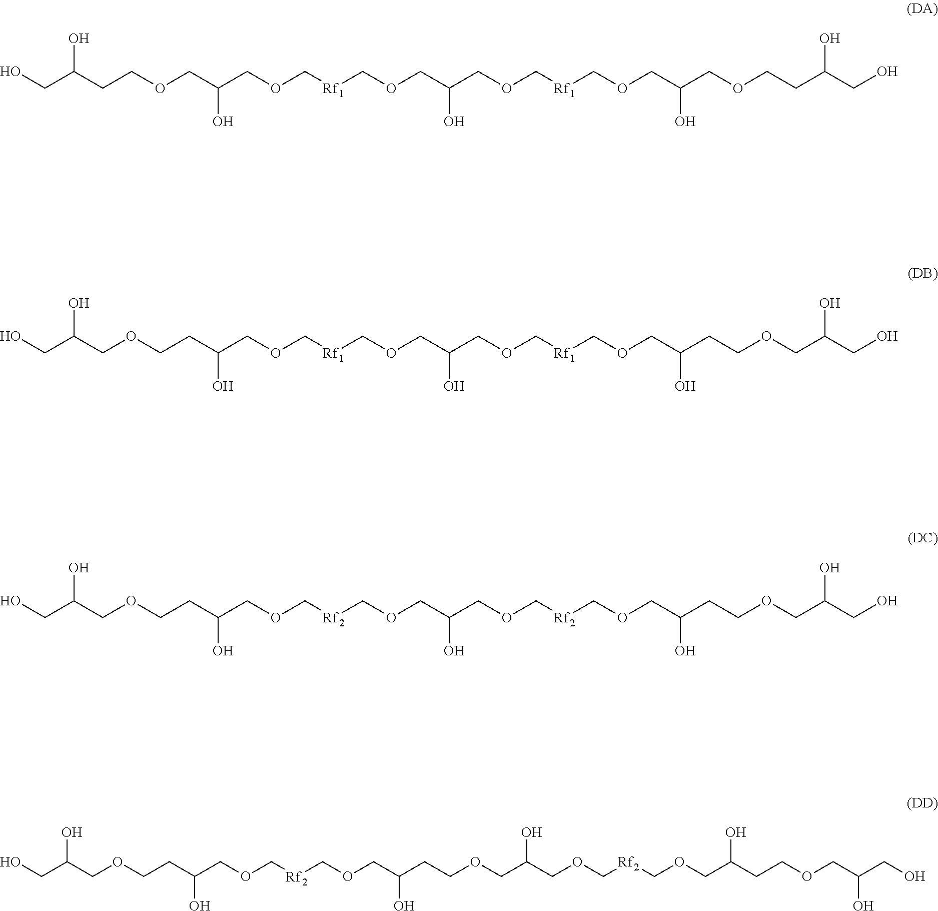

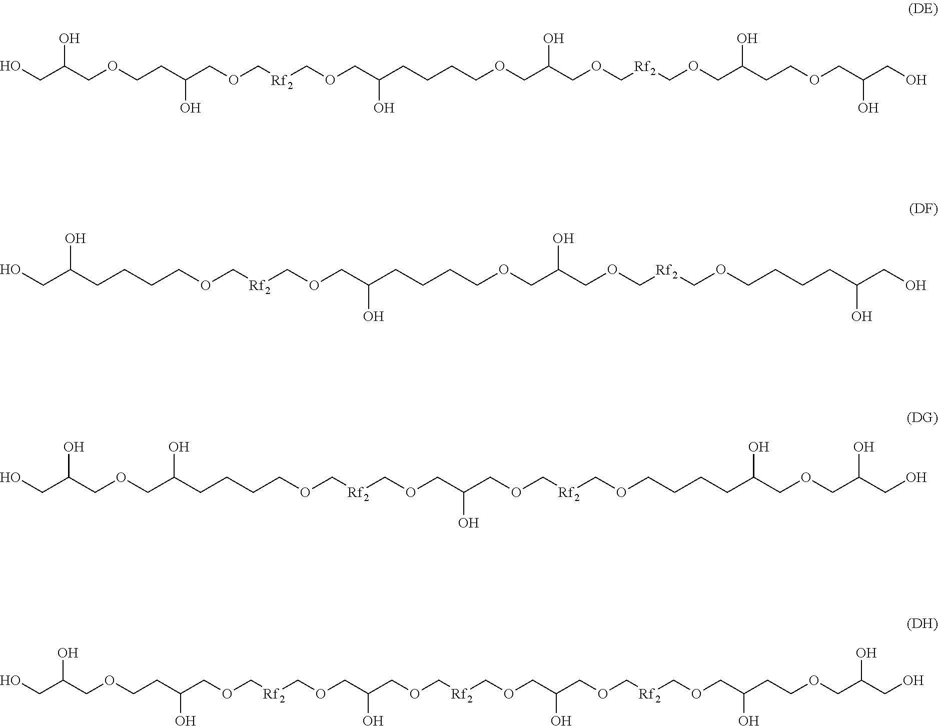

(In two Rf₁'s in Formula (DA), h and i represent an average degree of polymerization, h represents 1 to 20, and i represents 0. h in the two Rf₁'s may be the same as or different from each other.)

(In two Rf₁'s in Formula (DB), h and i represent an average degree of polymerization, h represents 1 to 20, and i represents 0. h in the two Rf₁'s may be the same as or different from each other.)

(In two Rf₂'s in Formula (DC), j represents an average degree of polymerization and represents 1 to 15. j's in the two Rf₂'s may be the same as or different from each other.)

(In two Rf₂'s in Formula (DD), j represents an average degree of polymerization and represents 1 to 15. j's in the two Rf₂'s may be the same as or different from each other.)

(In two Rf₂'s in Formula (DE), j represents an average degree of polymerization and represents 1 to 15. j's in the two Rf₂'s may be the same as or different from each other.)

(In two Rf₂'s in Formula (DF), j represents an average degree of polymerization and represents 1 to 15. j's in the two Rf₂'s may be the same as or different from each other.)

(In two Rf₂'s in Formula (DG), j represents an average degree of polymerization and represents 1 to 15. j's in the two Rf₂'s may be the same as or different from each other.)

(In three Rf₂'s in Formula (DH), j represents an average degree of polymerization and represents 1 to 15. j's in the three Rf₂'s may be partially or entirely the same as or different from each other.)

The number-average molecular weight (Mn) of the fluorine-containing ether compound of the present embodiment is preferably in a range of 500 to 10,000, more preferably in a range of 500 to 5,000, and particularly preferably in a range of 1,000 to 3,000. In a case where the number-average molecular weight thereof is 500 or greater, the lubricating layer formed of the lubricant containing the fluorine-containing ether compound according to the present embodiment has excellent heat resistance. The number-average molecular weight of the fluorine-containing ether compound is more preferably 1,000 or greater. In addition, in a case where the number-average molecular weight thereof is 10,000 or less, the viscosity of the fluorine-containing ether compound is appropriate, and a thin lubricating layer can be easily formed by being coated with a lubricant containing the fluorine-containing ether compound. The number-average molecular weight of the fluorine-containing ether compound is preferably 5,000 or less from the viewpoint that the viscosity is easy to handle in a case where the fluorine-containing ether compound is applied to a lubricant.

The number-average molecular weight (Mn) of the fluorine-containing ether compound is a value measured by ¹H-NMR and ¹⁹F-NMR using AVANCE III 400 (manufactured by Bruker BioSpin GmbH). Specifically, the number of repeating units of the PFPE chain is calculated from the integrated value measured by ¹⁹F-NMR, and the number-average molecular weight is determined. In the measurement of nuclear magnetic resonance (NMR), the sample is diluted with a solvent of hexafluorobenzene/d-acetone (4/1 v/v) and measured. As the reference of the ¹⁹F-NMR chemical shift, the peak of hexafluorobenzene is set to −164.7 ppm. As the reference of the ¹H-NMR chemical shift, the peak of acetone is set to 2.2 ppm.

In the fluorine-containing ether compound of the present embodiment, it is preferable that the polydispersity (ratio of weight-average molecular weight (Mw) to number-average molecular weight (Mn)) is set to 1.3 or less by fractionating the molecular weight by an appropriate method.

In the present embodiment, a method for fractionating the molecular weight is not particularly limited, and for example, molecular weight fractionation by a silica gel column chromatography method, a gel permeation chromatography (GPC) method, or the like, molecular weight fractionation by a supercritical extraction method, and the like can be used.

### “Production Method”

A method for producing the fluorine-containing ether compound of the present embodiment is not particularly limited, and the fluorine-containing ether compound can be produced by a known production method of the related art. The fluorine-containing ether compound of the present embodiment can be produced, for example, by the following production method.

[First Production Method (in Case where x Represents 1)]

(In case where R¹ and R⁴ are the same as each other and two R²'s are the same as each other)

A fluorine-based compound in which a hydroxymethyl group (—CH₂OH) is disposed at each of both ends of a perfluoropolyether chain corresponding to R² in Formula (1) is prepared.

Next, a hydroxy group of a hydroxymethyl group disposed at one end of the fluorine-based compound reacts with an epoxy compound (first raw material compound) having a group corresponding to R¹ in Formula (1) (=group corresponding to R⁴) (first reaction). In this manner, an intermediate compound 1 having a group corresponding to R¹ (=group corresponding to R⁴) at one end of the perfluoropolyether chain corresponding to R² is obtained.

In a case where the fluorine-based compound reacts with the first raw material compound, the hydroxy group included in the first raw material compound may be protected with an appropriate protective group and then may react with the fluorine-based compound.

As the first raw material compound in which two hydroxy groups of the 1,2-diol structure are protected, for example, compounds represented by Formulae (6-1) to (6-16) can be used as the epoxy compound having a group corresponding to R¹ in Formula (1) (=group corresponding to R⁴).

As the above-described first raw material compound, a compound having a group corresponding to R¹ in Formula (1) (=group corresponding to R⁴) and a leaving group (hereinafter, also simply referred to as “compound having a leaving group”) instead of the epoxy compound may be used. As the compound having a leaving group, a compound represented by Formulae (6-24) and (6-25) can be used. In Formulae (6-24) and (6-25), Ts represents a tosyl group. In Formula (6-25), MOM represents a methoxymethyl group.

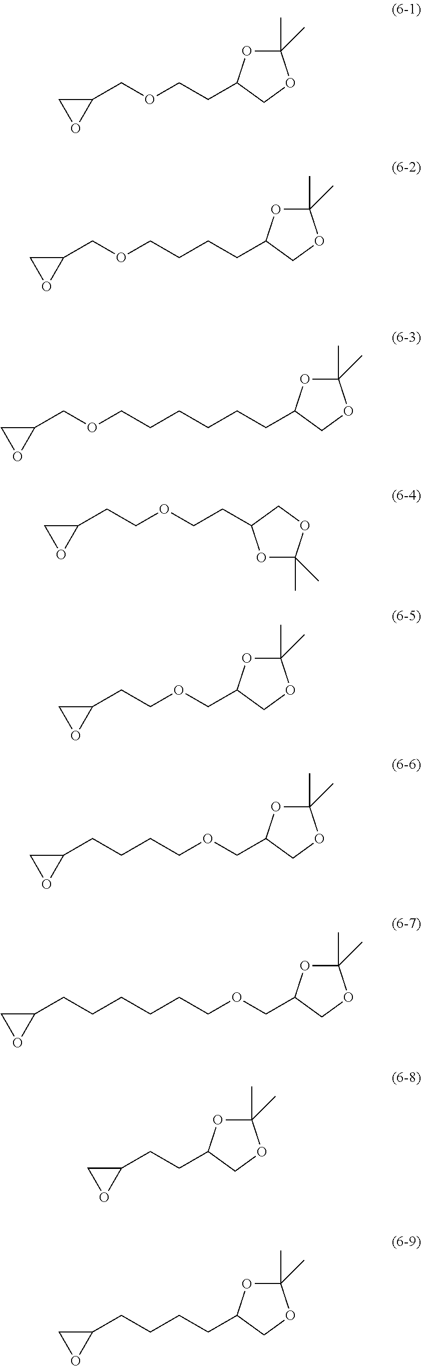

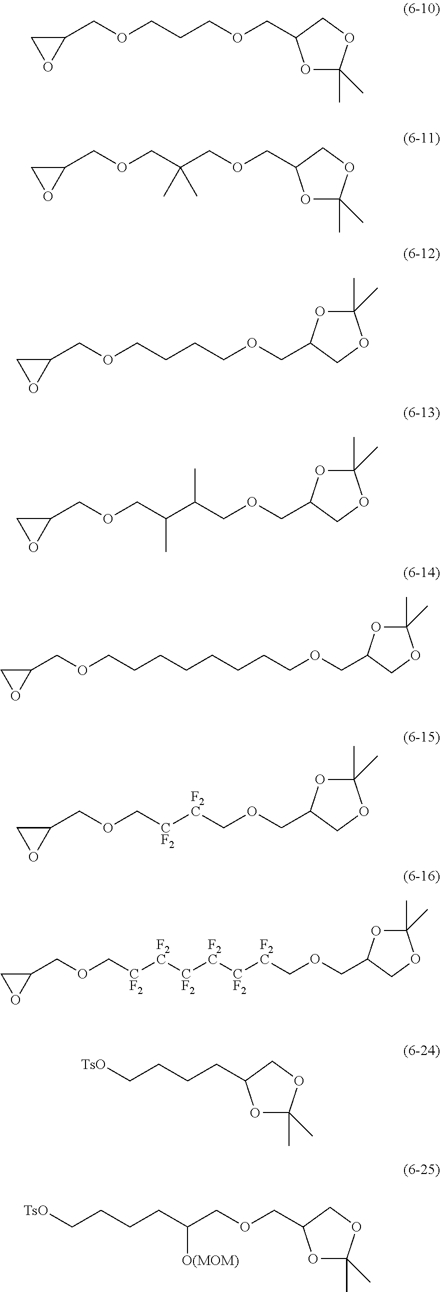

The epoxy compound represented by Formula (6-1), which is an example of the first raw material compound, can be produced by, for example, a method represented by Formula (7-1). That is, the epoxy compound can be produced by a method of reacting an alcohol compound having a part (X′) of X in Formula (2) of R¹ (=R⁴) and a portion corresponding to a 1,2-diol structure (—X′—CH(OH)—CH₂OH) bonded to the part with an epibromohydrin represented by Formula (8-1), which is a halogen compound having a portion corresponding to a part of X in Formula (2) and an epoxy group. As shown in Formula (7-1), a compound in which two hydroxy groups of a 1,2-diol structure are appropriately protected may be used as the alcohol compound.

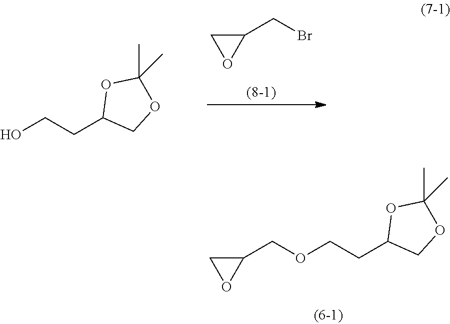

In addition, the epoxy compound represented by Formula (6-5), which is another example of the first raw material compound, can be produced by, for example, the method represented by Formula (7-2). That is, 3-butene-1-ol, which is an alcohol compound having a vinyl group and a portion corresponds to a part of X in Formula (2) reacts with a halogen compound having a part (X′) of X in Formula (2) of R¹ (=R⁴) and a portion corresponding to a 1,2-diol structure (—X′—CH(OH)—CH₂OH) bonded to the part (in the present embodiment, a halogen compound in which a hydroxy group of a solketal is brominated). Thereafter, the compound can be produced by a method of reacting the obtained compound with m-chloroperbenzoic acid (mCPBA) for oxidation. As shown in Formula (7-2), a compound in which two hydroxy groups of a 1,2-diol structure are appropriately protected may be used as the halogen compound.

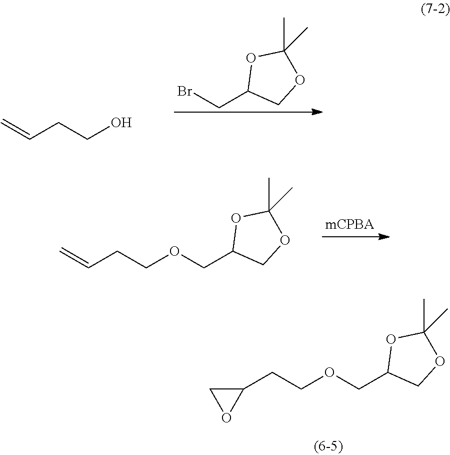

In addition, the epoxy compound represented by Formula (6-10), which is another example of the first raw material compound, can be produced by, for example, the method represented by Formula (7-3). That is, a halogen compound having a part (X′) of X in Formula (2) of R¹ (=R⁴) and a portion corresponding to a 1,2-diol structure (—X′—CH(OH)—CH₂OH) bonded to the part (in the present embodiment, a halogen compound in which a hydroxy group of a solketal is brominated) reacts with a diol compound having a portion corresponding to a part of X in Formula (2). Thereafter, the compound can be produced by a method of reacting the obtained alcohol compound with epibromohydrin represented by Formula (8-1), which is a halogen compound having a portion corresponding to a part of X in Formula (2) and an epoxy group. As shown in Formula (7-3), a compound in which two hydroxy groups of the 1,2-diol structure are appropriately protected, which is the same as the halogen compound used in the method represented by Formula (7-2), may be used as the halogen compound.

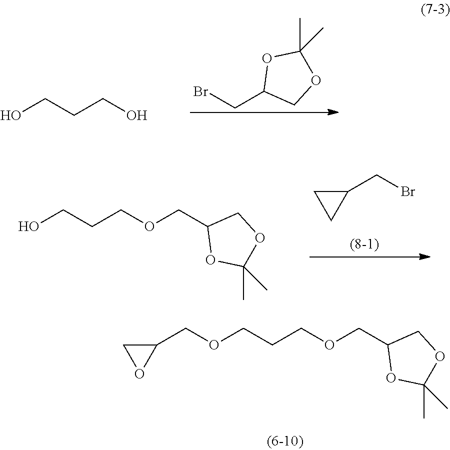

A commercially available product may be purchased and used as the epoxy compound (or a compound having a leaving group) having a group corresponding to R which is the first raw material compound (=group corresponding to R⁴).

Thereafter, a hydroxy group of the hydroxymethyl group disposed at one end of an intermediate compound 1 generated in the first reaction described above reacts with a compound having an epoxy group at one end of a portion corresponding to R³ in Formula (1) and having a halogen bonded to the other end or a compound (second raw material compound) having epoxy groups at both ends of a portion corresponding to R³ in Formula (1) (second reaction).

As the second raw material compound which is a compound in which one end of a portion corresponding to R³ in Formula (1) has an epoxy group and halogen is bonded to the other end or a compound in which both ends of a portion corresponding to R³ in Formula (1) have an epoxy group, for example, compounds represented by Formulae (8-1) to (8-12) can be used. In Formula (8-7), THP represents a tetrahydropyranyl group.

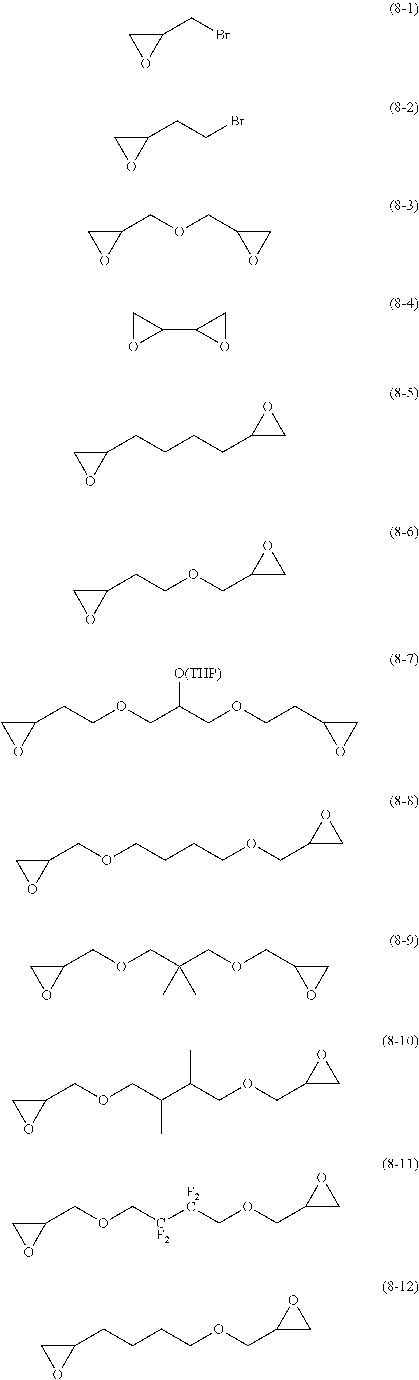

The second raw material compound can be produced, for example, by the following method. That is, the second raw material compound can be produced by a method of reacting a diol corresponding to a part of a linking group represented by R³ with epibromohydrin in a molar amount twice the molar amount of the diol.

For example, in a case of producing a compound represented by Formula (8-8), which is an example of the second raw material compound, the compound can be produced by a method of reacting 1,4-butanediol with epibromohydrin represented by Formula (8-1) in a molar amount twice the molar amount of 1,4-butanediol, as shown in Formula (9-1).

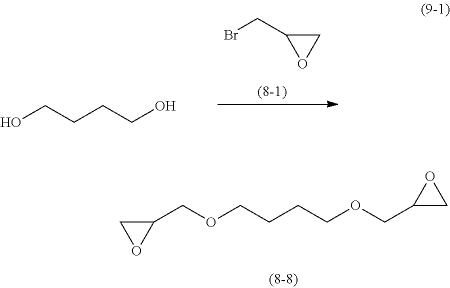 [SMILES: BrCC1CO1 ; OCCCCO ; C(CCOCC1CO1)COCC1CO1]

The second raw material compound may be produced by the following method. That is, the halogen compound having an epoxy group, which corresponds to a part of the linking group represented by R³, and the alcohol having an alkenyl group, which corresponds to a part of the linking group represented by R³, are subjected to an addition reaction. In this case, the alcohol having an alkenyl group in a molar amount twice the molar amount of a halogen compound reacts with the halogen compound. Thereafter, the compound can be produced by a method of reacting the obtained compound with m-chloroperbenzoic acid (mCPBA) for oxidation. Before the compound obtained by the addition reaction reacts with m-chloroperbenzoic acid (mCPBA) for oxidation, a hydroxy group generated by the addition reaction may be protected by a known method.

For example, the compound represented by Formula (8-7), which is another example of the second raw material compound, can be produced by a method of performing an addition reaction on epibromohydrin represented by Formula (8-1) with 3-butene-1-ol in a molar amount twice the molar amount of the epibromohydrin as shown in Formula (9-2), protecting a hydroxy group generated by the addition reaction using dihydropyran (DHP), and then reacting the obtained compound with m-chloroperbenzoic acid (mCPBA) for oxidation. In Formula (9-2), THP represents a tetrahydropyranyl group.

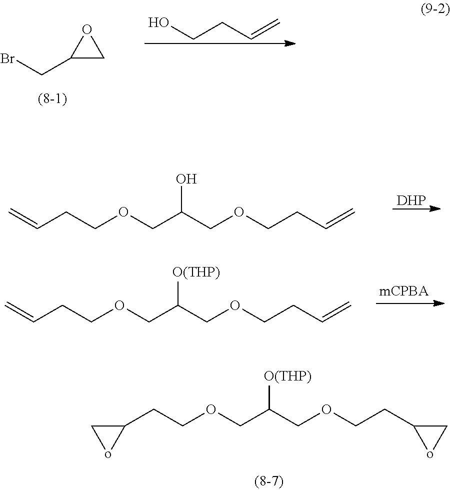

In a case where the compound obtained after the above-described second reaction has a hydroxy group protected by using a protective group, a deprotection reaction is carried out using a known method. By performing the above-described step, a fluorine-containing ether compound in which x in Formula (1) represents 1, R¹ and R⁴ are the same as each other, and two R²'s are the same as each other can be produced.

[Second Production Method (in Case where x Represents 1)]

(In case where R¹ and R⁴ are different from each other and/or two R²'s are different from each other)

First, the hydroxy group at one end of the fluorine-based compound in which the hydroxymethyl group is disposed at each of both ends of the perfluoropolyether chain corresponding to R² on the R¹ side reacts with the epoxy compound (or the compound having a leaving group) having a group corresponding to R¹ to obtain an intermediate compound 1a (first reaction).

As the epoxy compound (or the compound having a leaving group) having a group corresponding to R¹, for example, any compound selected from the above-described first raw material compounds can be used.

Next, the hydroxy group at one end of the fluorine-based compound in which the hydroxymethyl group is disposed at each of both ends of the perfluoropolyether chain corresponding to R² on the R⁴ side reacts with the epoxy compound (or the compound having a leaving group) having a group corresponding to R⁴ to obtain an intermediate compound 1b (second reaction).

In the second production method, in a case of producing the fluorine-containing ether compound in which R¹ and R⁴ are the same as each other (that is, R¹ and R⁴ represent the same end group represented by Formula (2)), the same compound as the epoxy compound (or the compound having a leaving group) having a group corresponding to R¹ is used as the epoxy compound (or the compound having a leaving group) having a group corresponding to R⁴.

In the second production method, in a case of producing the fluorine-containing ether compound in which R¹ and R⁴ are different from each other and both R¹ and R⁴ represent an end group represented by Formula (2), for example, any compound selected from the above-described first raw material compounds, which is a compound different from the epoxy compound (or the compound having a leaving group) having a group corresponding to R¹ can be used as the epoxy compound (or the compound having a leaving group) having a group corresponding to R⁴.

In addition, in a case of producing the fluorine-containing ether compound in which R¹ and R⁴ are different from each other, R¹ represents an end group represented by Formula (2), and R⁴ represents an end group that does not correspond to Formula (2) (only one of R¹ or R⁴ represents an end group represented by Formula (2)) in the second production method, an epoxy compound (third raw material compound) in which a portion corresponding to R⁴ of the intermediate compound 1b obtained after the reaction does not correspond to a part of Formula (2) is used as the epoxy compound having a group corresponding to R⁴.

As the third raw material compound, for example, an epoxy compound represented by any of Formulae (6-17) to (6-23) can be used. In Formulae (6-17), (6-18), and (6-21) to (6-23), THP represents a tetrahydropyranyl group.

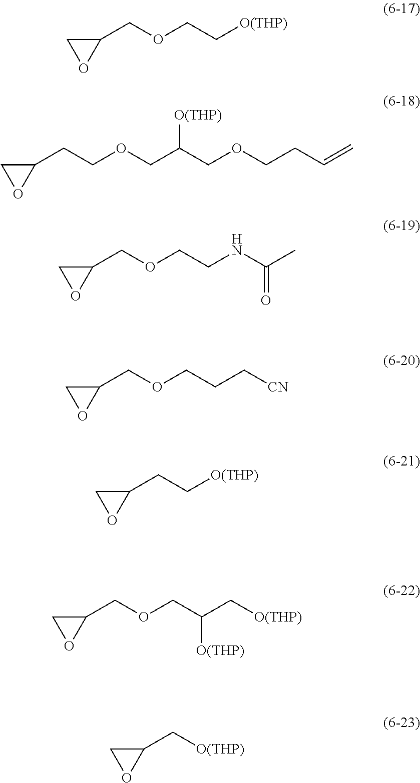

Next, the intermediate compound 1a obtained in the first reaction reacts with any of the compounds selected from the second raw material compounds described above. In this manner, an intermediate compound 2a in which one end of the perfluoropolyether chain corresponding to R² on the R¹ side has a group corresponding to R¹ and the other end has an epoxy group corresponding to R³ is produced (third reaction).

In the third reaction, a compound which has an epoxy group at one end of a portion corresponding to R³ in Formula (1) and has an alkenyl group bonded to the other end may be used instead of the second raw material compound, and a double bond of the generated compound may be oxidized to produce an intermediate compound 2a having an epoxy group.

Subsequently, the intermediate compound 1b obtained by the second reaction reacts with the intermediate compound 2a obtained by the third reaction (fourth reaction).

In a case where the compound obtained after the above-described fourth reaction has a hydroxy group protected by using a protective group, a deprotection reaction is carried out using a known method. By performing the above-described step, a fluorine-containing ether compound in which x in Formula (1) represents 1 and R¹ and R⁴ are different from each other and/or two R²'s are different from each other can be produced.

[Third Production Method (in Case where x Represents 2)]

(In case where R¹ and R⁴ are the same as each other, two R³'s are the same as each other, and R² on the R¹ side and R² on the R⁴ side are the same as each other)

First, in the same manner as in the first production method, an intermediate compound 1 having a group corresponding to R¹ (=group corresponding to R⁴) at one end of the perfluoropolyether chain corresponding to R² on the R¹ side and the R⁴ side is obtained (first reaction).

Next, a fluorine-based compound in which a hydroxymethyl group (—CH₂OH) is disposed at each of both ends of the perfluoropolyether chain corresponding to R² at the center of the molecule in Formula (1) is prepared. Next, a hydroxy group of a hydroxymethyl group disposed at both ends of the fluorine-based compound reacts with any compound selected from the second raw material compounds described above. In this manner, an intermediate compound 3a having an epoxy group corresponding to R³ at both ends of the perfluoropolyether chain corresponding to R² at the center of the molecule is obtained (second reaction).

In the second reaction, a compound which has an epoxy group at one end of a portion corresponding to R³ in Formula (1) and has an alkenyl group bonded to the other end may be used instead of the second raw material compound, and a double bond of the generated compound may be oxidized to produce an intermediate compound 3a having an epoxy group at both ends.

Thereafter, a hydroxy group of the hydroxymethyl group disposed at one end of the intermediate compound 1 obtained in the first reaction reacts with an epoxy group disposed at both ends of the intermediate compound 3a (third reaction).

In a case where the compound obtained after the above-described third reaction has a hydroxy group protected by using a protective group, a deprotection reaction is carried out using a known method. By performing the above-described step, a fluorine-containing ether compound in which x in Formula (1) represents 2, R¹ and R⁴ are the same as each other, two R³'s are the same as each other, and R² on the R¹ side and R² on the R⁴ side are the same as each other can be produced.

[Fourth Production Method (in Case where x Represents 2)]

(In case where R³ on the R¹ side and R³ on the R⁴ side are the same as each other and R¹ and R⁴ are different from each other and/or R² on the R¹ side and R² on the R⁴ side are different from each other)

In the first reaction of the third production method, the intermediate compound 1a and the intermediate compound 1b in the second production method are obtained instead of the intermediate compound 1. Next, the intermediate compound 3a is obtained in the same manner as in the second reaction of the third production method. Further, the epoxy groups disposed at both ends of the intermediate compound 3a sequentially react with the intermediate compound 1a and the intermediate compound 1b.

In a case where the compound obtained after the above-described step has a hydroxy group protected by using a protective group, a deprotection reaction is carried out using a known method. By performing the above-described step, a fluorine-containing ether compound in which x in Formula (1) represents 2, R³ on the R¹ side and R³ on the R⁴ side are the same as each other, and R¹ and R⁴ are different from each other and/or R² on the R¹ side and R² on the R⁴ side are different from each other can be produced.

[Fifth Production Method (in Case where x Represents 2)]

(In case where R³ on R¹ side and R⁴ on R³ side are different from each other, R¹ and R⁴ are the same as each other, and R² on R¹ side and R² on R⁴ side are the same as each other)

In the second reaction of the third production method, a fluorine-based compound in which a hydroxymethyl group (—CH₂OH) is disposed at each of both ends of the perfluoropolyether chain corresponding to R² at the center of the molecule in Formula (1) reacts with any one compound having a portion corresponding to R³ on the R¹ side selected from the second raw material compound described above. Further, the obtained compound reacts with any one compound having a portion corresponding to R³ on the R⁴ side selected from the above-described second raw material compounds to obtain an intermediate compound 3b.

Thereafter, a third reaction is carried out in the same manner as in the third production method except that the intermediate compound 3b is used instead of the intermediate compound 3a.

In a case where the compound obtained after the above-described third reaction has a hydroxy group protected by using a protective group, a deprotection reaction is carried out using a known method. By performing the above-described step, a fluorine-containing ether compound in which x in Formula (1) represents 2, R³ on the R¹ side and R³ on the R⁴ side are different from each other, R¹ and R⁴ are the same as each other, and R² on the R¹ side and R² on the R⁴ side are the same as each other can be produced.

A fluorine-containing ether compound in which x in Formula (1) represents 2 and the perfluoropolyether chain corresponding to R² at the center of the molecule is the same as R² on the R¹ side and/or R² on the R⁴ side, and a fluorine-containing ether compound in which the perfluoropolyether chain thereof is different from R² on the R¹ side and/or R² on the R⁴ side can be produced by appropriately selecting the kind of the fluorine-based compound having a perfluoropolyether chain corresponding to R² at the center of the molecule, which is used in the third production method to the fifth production method described above.

Therefore, the fluorine-based compound having a perfluoropolyether chain corresponding to R² at the center of the molecule, which is used in the third production method to the fifth production method for producing the fluorine-containing ether compound in which x in Formula (1) represents 2, may be the same as or different from the fluorine-based compound having a perfluoropolyether chain corresponding to the other R².

In addition, the method for producing the fluorine-containing ether compound of the present embodiment is not limited to the above-described first to fifth production methods, and for example, the following methods may be used.

A fluorine-based compound in which a hydroxymethyl group (—CH₂OH) is disposed at both ends of a perfluoropolyether chain corresponding to R² in Formula (1) (in a case where x represents 2, a perfluoropolyether chain corresponding to R² on the R¹ side and/or R² on the R⁴ side) is prepared.

Next, a hydroxy group of the hydroxymethyl group disposed at one end of the fluorine-based compound reacts with a halogen compound having a vinyl group at the end. In this manner, an intermediate compound 1c having a vinyl group is obtained at one end of the chain structure having a perfluoropolyether chain.

Thereafter, the first production method to the fifth production method are carried out to the final reaction by using the intermediate compound 1c instead of the intermediate compound 1, the intermediate compound 1a, or the intermediate compound 1b in the first production method to the fifth production method. In this manner, a compound having a vinyl group is obtained at one or both ends of the chain structure having a perfluoropolyether chain.

Subsequently, the vinyl group at one or both ends of the obtained compound is oxidized to obtain a compound having an epoxy group at one or both ends of the chain structure having a perfluoropolyether chain. Thereafter, the epoxy group is converted into a 1,2-diol by performing a ring-opening reaction of the epoxy group disposed at one or both ends of the obtained compound.

The fluorine-containing ether compound represented by Formula (1) can also be produced by performing the above-described step.

### [Lubricant for Magnetic Recording Medium]

A lubricant for a magnetic recording medium according to the present embodiment contains the fluorine-containing ether compound represented by Formula (1).

In the lubricant of the present embodiment, as long as the characteristics are not impaired by containing the fluorine-containing ether compound represented by Formula (1), known materials used as a material for the lubricant can be used in the form of a mixture as necessary.

Specific examples of the known materials include, for example, FOMBLIN (registered trademark) ZDIAC, FOMBLIN ZDEAL, and FOMBLIN AM-2001 (all manufactured by Solvay Solexis), and Moresco A20H (manufactured by Moresco). The known materials used in the form of a mixture with the lubricant according to the present embodiment has a number-average molecular weight of preferably 1000 to 10000.

In a case where the lubricant of the present embodiment contains other materials of the fluorine-containing ether compound represented by Formula (1), the content of the fluorine-containing ether compound represented by Formula (1) in the lubricant of the present embodiment is preferably 70% by mass or greater, more preferably 90% by mass or greater, and still more preferably 95% by mass or greater.

Since the lubricant of the present embodiment contains the fluorine-containing ether compound represented by Formula (1), a lubricating layer having excellent corrosion resistance and a high pickup suppression effect can be formed.

### [Magnetic Recording Medium]

The magnetic recording medium according to the present embodiment is formed by sequentially providing at least a magnetic layer, a protective layer, and a lubricating layer on a substrate.

In the magnetic recording medium according to the present embodiment, one or two or more underlayers can be provided between the substrate and the magnetic layer as necessary. In addition, at least one of an adhesive layer or a soft magnetic layer can be provided between the underlayer and the substrate.

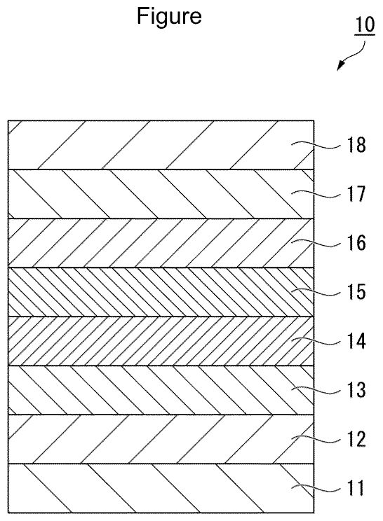

FIG. 1 is a schematic cross-sectional view showing an embodiment of the magnetic recording medium according to the present invention.

The magnetic recording medium 10 according to the present embodiment has a structure in which an adhesive layer 12, a soft magnetic layer 13, a first underlayer 14, a second underlayer 15, a magnetic layer 16, a protective layer 17, and a lubricating layer 18 are sequentially provided on a substrate 11.

### “Substrate”

As the substrate 11, for example, a non-magnetic substrate on which a film made of NiP or a NiP alloy is formed on a base made of a metal or an alloy material such as Al or an Al alloy can be used.

In addition, as the substrate 11, a non-magnetic substrate made of a non-metal material such as glass, ceramics, silicon, silicon carbide, carbon, or a resin may be used, or a non-magnetic substrate in which a film of NiP or a NiP alloy is formed on a base made of these non-metal materials may be used.

### “Adhesive Layer”

The adhesive layer 12 prevents the progression of corrosion of the substrate 11 in a case where the substrate 11 and the soft magnetic layer 13 provided on the adhesive layer 12 are disposed in contact with each other.

The material of the adhesive layer 12 can be appropriately selected from, for example, Cr, a Cr alloy, Ti, a Ti alloy, CrTi, NiAl, and an AlRu alloy. The adhesive layer 12 can be formed by, for example, a sputtering method.

### “Soft Magnetic Layer”

It is preferable that the soft magnetic layer 13 has a structure in which a first soft magnetic film, an intermediate layer formed of a Ru film, and a second soft magnetic film are sequentially laminated. That is, it is preferable that the soft magnetic layer 13 has a structure in which the upper and lower soft magnetic films of the intermediate layer are antiferro-coupled (AFC) by interposing the intermediate layer formed of a Ru film between the two layers of the soft magnetic films.

Examples of the material of the first soft magnetic film and the second soft magnetic film include a CoZrTa alloy and a CoFe alloy.

It is preferable to add any of Zr, Ta, or Nb to the CoFe alloy used in the first soft magnetic film and the second soft magnetic film. In this manner, the amorphization of the first soft magnetic film and the second soft magnetic film is promoted. As a result, it is possible to improve the aligning properties of the first underlayer (seed layer) and to reduce the floating amount of the magnetic head.

The soft magnetic layer 13 can be formed by, for example, a sputtering method.

### “First Underlayer”

The first underlayer 14 is a layer that controls the alignment and the crystal size of the second underlayer 15 and the magnetic layer 16 provided thereon.

Examples of the first underlayer 14 include a Cr layer, a Ta layer, a Ru layer, a CrMo alloy layer, a CoW alloy layer, a CrW alloy layer, a CrV alloy layer, and a CrTi alloy layer.

The first underlayer 14 can be formed by, for example, a sputtering method.

### “Second Underlayer”

The second underlayer 15 is a layer in which the alignment of the magnetic layer 16 is controlled to be satisfactory. The second underlayer 15 is preferably a layer formed of Ru or a Ru alloy.

The second underlayer 15 may consist of a single layer or a plurality of layers. In a case where the second underlayer 15 consists of a plurality of layers, all the layers may be formed of the same material, or at least one layer may be formed of a different material.

The second underlayer 15 can be formed by, for example, a sputtering method.

### “Magnetic Layer”

The magnetic layer 16 is formed of a magnetic film in which a magnetization easy axis is oriented in a direction perpendicular or horizontal to the substrate surface. The magnetic layer 16 is a layer containing Co and Pt. The magnetic layer 16 may be a layer containing an oxide, Cr, B, Cu, Ta, Zr, or the like in order to improve the SNR characteristics.

Examples of the oxide contained in the magnetic layer 16 include SiO₂, SiO, Cr₂O₃, CoO, Ta₂O₃, and TiO₂.

The magnetic layer 16 may consist of one layer or a plurality of magnetic layers formed of materials having different compositions.

For example, in a case where the magnetic layer 16 consists of three layers of a first magnetic layer, a second magnetic layer, and a third magnetic layer which are laminated in this order from the bottom, it is preferable that the first magnetic layer has a granular structure formed of a material containing Co, Cr, and Pt and further containing an oxide. As the oxide contained in the first magnetic layer, for example, an oxide of Cr, Si, Ta, Al, Ti, Mg, or Co is preferably used. Among these, TiO₂, Cr₂O₃, SiO₂, or the like can be particularly suitably used. In addition, it is preferable that the first magnetic layer is formed of a composite oxide to which two or more kinds of oxides are added. Among these, Cr₂O₃—SiO₂, Cr₂O₃—TiO₂, SiO₂—TiO₂, or the like can be particularly suitably used. The first magnetic layer can contain one or more kinds of elements selected from B, Ta, Mo, Cu, Nd, W, Nb, Sm, Tb, Ru, and Re in addition to Co, Cr, Pt, and an oxide.

The same material as that of the first magnetic layer can be used for the second magnetic layer. It is preferable that the second magnetic layer has a granular structure.

It is preferable that the third magnetic layer has a non-granular structure formed of a material containing Co, Cr, and Pt and not containing an oxide. The third magnetic layer can contain one or more elements selected from B, Ta, Mo, Cu, Nd, W, Nb, Sm, Tb, Ru, Re, and Mn in addition to Co, Cr, and Pt.

In a case where the magnetic layer 16 is formed of a plurality of magnetic layers, it is preferable to provide a non-magnetic layer between the adjacent magnetic layers. In a case where the magnetic layer 16 is formed of three layers of a first magnetic layer, a second magnetic layer, and a third magnetic layer, it is preferable to provide a non-magnetic layer between the first magnetic layer and the second magnetic layer and between the second magnetic layer and the third magnetic layer.

As the non-magnetic layer provided between adjacent magnetic layers of the magnetic layer 16, for example, Ru, a Ru alloy, a CoCr alloy, or a CoCrX1 alloy (X1 represents one or two or more kinds of elements selected from Pt, Ta, Zr, Re, Ru, Cu, Nb, Ni, Mn, Ge, Si, O, N, W, Mo, Ti, V, and B) can be suitably used.

It is preferable that, as the non-magnetic layer provided between adjacent magnetic layers of the magnetic layer 16, an alloy material containing an oxide, a metal nitride, or a metal carbide is used. Specifically, for example, SiO₂, Al₂O₃, Ta₂O₅, Cr₂O₃, MgO, Y₂O₃, or TiO₂ can be used as the oxide. As the metal nitride, for example, AlN, Si₃N₄, TaN, or CrN can be used. As the metal carbide, for example, TaC, BC, or SiC can be used.

The non-magnetic layer can be formed by, for example, a sputtering method.

In order to achieve a higher recording density, it is preferable that the magnetic layer 16 is a magnetic layer for perpendicular magnetic recording in which a magnetization easy axis is oriented in a direction perpendicular to the substrate surface. The magnetic layer 16 may be a magnetic layer for in-plane magnetic recording.

The magnetic layer 16 may be formed by any of the known methods in the related art, such as an evaporation method, an ion beam sputtering method, and a magnetron sputtering method. The magnetic layer 16 is usually formed by a sputtering method.

### “Protective Layer”

The protective layer 17 protects the magnetic layer 16. The protective layer 17 may be formed of one layer or a plurality of layers. Examples of the material of the protective layer 17 include carbon, carbon containing nitrogen, and silicon carbide. As the protective layer 17, a carbon-based protective layer can be preferably used, and an amorphous carbon protective layer is particularly preferable. In a case where the protective layer 17 is a carbon-based protective layer, the interaction with the polar group (particularly, the hydroxy group) contained in the fluorine-containing ether compound in the lubricating layer 18 is further enhanced, which is preferable.

The adhesion force between the carbon-based protective layer and the lubricating layer 18 can be controlled by forming the carbon-based protective layer with hydrocarbon and/or nitrogenated carbon and adjusting the hydrogen content and/or the nitrogen content in the carbon-based protective layer. The hydrogen content in the carbon-based protective layer is preferably in a range of 3% by atom to 20% by atom in a case of being measured by a hydrogen forward scattering method (HFS). In addition, the nitrogen content in the carbon-based protective layer is preferably in a range of 4% by atom to 15% by atom in a case of being measured by X-ray photoelectron spectroscopy (XPS).

The hydrogen and/or nitrogen contained in the carbon-based protective layer are not necessarily uniformly contained in the entire carbon-based protective layer. It is suitable that the carbon-based protective layer is, for example, a composition gradient layer in which nitrogen is contained on the lubricating layer 18 side of the protective layer 17 and hydrogen is contained on the magnetic layer 16 side of the protective layer 17. In this case, the adhesion force between the magnetic layer 16, the lubricating layer 18, and the carbon-based protective layer is further improved.

The film thickness of the protective layer 17 is preferably in a range of 1 nm to 7 nm. In a case where the film thickness of the protective layer 17 is 1 nm or greater, the performance as the protective layer 17 can be sufficiently obtained. It is preferable that the film thickness of the protective layer 17 is 7 nm or less from the viewpoint of reducing the film thickness of the protective layer 17.

As a film forming method of the protective layer 17, a sputtering method using a target material containing carbon, a CVD (chemical vapor deposition) method using a hydrocarbon raw material such as ethylene or toluene, an ion beam deposition (IBD) method, or the like can be used.

In a case where the carbon-based protective layer is formed as the protective layer 17, the carbon-based protective layer can be formed, for example, by a DC magnetron sputtering method. In particular, in a case where a carbon-based protective layer is formed as the protective layer 17, it is preferable to form an amorphous carbon protective layer by a plasma CVD method. The amorphous carbon protective layer formed by the plasma CVD method has a uniform surface and small roughness.

### “Lubricating Layer”

The lubricating layer 18 prevents contamination of the magnetic recording medium 10. In addition, the lubricating layer 18 reduces a frictional force of the magnetic head of the magnetic recording and reproducing device that slides on the magnetic recording medium 10 to improve the durability of the magnetic recording medium 10.

As shown in FIG. 1, the lubricating layer 18 is formed in contact with the protective layer 17. The lubricating layer 18 is formed by applying the lubricant for a magnetic recording medium according to the embodiment described above onto the protective layer 17. Therefore, the lubricating layer 18 contains the above-described fluorine-containing ether compound.

In a case where the protective layer 17 disposed below the lubricating layer 18 is a carbon-based protective layer, the lubricating layer 18 is particularly bonded to the protective layer 17 with a high binding force. As a result, even in a case where the thickness of the lubricating layer 18 is small, the magnetic recording medium 10 in which the surface of the protective layer 17 is coated at a high coating rate is likely to be obtained, and the contamination of the surface of the magnetic recording medium 10 can be effectively prevented.

The average film thickness of the lubricating layer 18 is preferably in a range of 0.5 nm (5 Å) to 2.0 nm (20 Å) and more preferably in a range of 0.5 nm (5 Å) to 1.2 nm (12 Å). In a case where the average film thickness of the lubricating layer 18 is 0.5 nm or greater, the lubricating layer 18 is formed with a uniform film thickness without being island-like or mesh-like. Therefore, the surface of the protective layer 17 can be coated with the lubricating layer 18 at a high coating rate. In addition, in a case where the average film thickness of the lubricating layer 18 is set to 2.0 nm or less, the lubricating layer 18 can be sufficiently thinned, and the floating amount of the magnetic head can be sufficiently reduced.

### “Method for Forming Lubricating Layer”

Examples of a method of forming the lubricating layer 18 include a method of preparing a magnetic recording medium in the middle of production in which each layer up to the protective layer 17 is formed on the substrate 11, coating the protective layer 17 with a lubricating layer forming solution, and drying the solution.

The lubricating layer forming solution is obtained by dispersing and dissolving the lubricant for a magnetic recording medium according to the embodiment described above in a solvent as necessary, and setting the viscosity and the concentration suitable for a coating method.

Examples of a solvent used in the lubricating layer forming solution include a fluorine-based solvent such as VERTREL (registered trademark) XF (trade name, manufactured by DuPont Mitsui Fluorochemicals Co., Ltd.) and/or ASAHIKLIN (registered trademark) AE-3000 (trade name, manufactured by AGC Inc.).

A coating method for the lubricating layer forming solution is not particularly limited, and examples thereof include a spin coating method, a spraying method, a paper coating method, and a dipping method.

In a case where the dipping method is used, for example, the following method can be used. First, the substrate 11 on which each layer up to the protective layer 17 is formed is immersed in a lubricating layer forming solution placed in an immersion tank of a dip coating device. Next, the substrate 11 is pulled up from the immersion tank at a predetermined speed. In this manner, the lubricating layer forming solution is applied onto the surface of the protective layer 17 of the substrate 11.

By using the dipping method, the lubricating layer forming solution can be uniformly applied to the surface of the protective layer 17, and the lubricating layer 18 can be formed on the protective layer 17 with a uniform film thickness.

In the present embodiment, it is preferable to perform a thermal treatment on the substrate 11 on which the lubricating layer 18 has been formed. By performing the thermal treatment, the adhesion between the lubricating layer 18 and the protective layer 17 is improved, and the adhesion force between the lubricating layer 18 and the protective layer 17 is improved.

The thermal treatment temperature is preferably in a range of 100° C. to 180° C. and more preferably in a range of 100° C. to 160° C. In a case where the thermal treatment temperature is 100° C. or higher, the effect of improving the adhesion between the lubricating layer 18 and the protective layer 17 is sufficiently obtained. In addition, thermal decomposition of the lubricating layer 18 due to the thermal treatment can be prevented by setting the thermal treatment temperature to 180° C. or lower. The thermal treatment time can be appropriately adjusted according to the thermal treatment temperature and is preferably 10 minutes to 120 minutes.

In the present embodiment, in order to further improve the adhesion force of the lubricating layer 18 to the protective layer 17, a treatment of irradiating the lubricating layer 18 with ultraviolet rays (UV) may be performed before or after the thermal treatment.

The magnetic recording medium 10 according to the present embodiment is formed by sequentially providing at least the magnetic layer 16, the protective layer 17, and the lubricating layer 18 on the substrate 11. In the magnetic recording medium 10 according to the present embodiment, the lubricating layer 18 containing the above-described fluorine-containing ether compound is formed in contact with the protective layer 17. The lubricating layer 18 has satisfactory corrosion resistance and a high pickup suppression effect even in a case where the film thickness is small. Accordingly, the magnetic recording medium 10 according to the present embodiment has excellent reliability, particularly excellent corrosion resistance, pickup suppression, and durability. Therefore, the magnetic recording medium 10 according to the present embodiment can contribute to the reduction of the magnetic spacing, and the floating amount of the magnetic head can be reduced (for example, 10 nm or less), and the magnetic recording medium 10 can stably operate for a long period of time even in a severe environment due to the diversification of applications. Therefore, the magnetic recording medium 10 according to the present embodiment is particularly suitable as a magnetic disk mounted on a magnetic disk apparatus of a load unload (LUL) system.

### EXAMPLES

Hereinafter, the present invention will be described in more detail with reference to examples and comparative examples. Further, the present invention is not limited to the following examples.

### Example 1

A compound represented by Formula (AA) was obtained by the following method.

A 100 mL eggplant flask was charged with 12.5 g of a compound represented by HOCH₂CF₂O(CF₂CF₂O)ₕ(CF₂O)ᵢCF₂CH₂OH (h representing the average degree of polymerization in the formula represent 4.5, and i representing the average degree of polymerization represent 4.5) (number-average molecular weight: 1,000, molecular weight distribution: 1.1), 2.70 g of a compound (first raw material compound) represented by Formula (6-1), and 12 mL of t-butanol in a nitrogen gas atmosphere, and the mixture was stirred at room temperature until the mixture was uniform. 1.10 g of potassium tert-butoxide was added to the mixture, and the mixture was stirred at 70° C. for 16 hours to carry out a reaction.

The compound represented by Formula (6-1) was produced by the method represented by Formula (7-1). That is, a 1,2-diol moiety of 1,2,4-butanetriol was protected with acetone. Thereafter, the compound was synthesized by reacting the hydroxy group of the obtained compound with epibromohydrin represented by Formula (8-1).

The reaction product obtained after the reaction was cooled to 25° C., transferred to a separatory funnel charged with 100 mL of water, and extracted three times with 100 mL of ethyl acetate. The organic layer was washed with water and dewatered with anhydrous sodium sulfate. The drying agent was separated by filtration, and the filtrate was concentrated, and the residues were purified by silica gel column chromatography, thereby obtaining 7.21 g of a compound represented by Formula (10-1) as an intermediate compound 1.

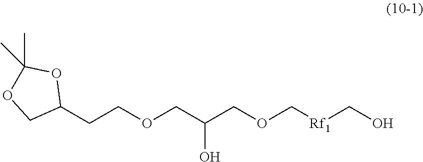

(Rf₁ in Formula (10-1) is a PFPE chain represented by Formula (5-1). In Rf₁, h representing the average degree of polymerization represents 4.5, and i representing the average degree of polymerization represents 4.5.)

Next, a 100 mL eggplant flask was charged with 7.21 g of the compound represented by Formula (10-1), which was an intermediate compound 1 obtained above, 0.55 g of epibromohydrin (second raw material compound) represented by Formula (8-1), and 10 mL of t-butanol in a nitrogen gas atmosphere, and the mixture was stirred at room temperature until the mixture was uniform. 0.72 g of potassium tert-butoxide was added to the uniform liquid, and the mixture was stirred at 70° C. for 23 hours to carry out a reaction.

The reaction solution obtained after the reaction was cooled to room temperature, 10 g of a 10% hydrogen chloride/methanol solution (hydrogen chloride-methanol reagent (5-10%) manufactured by Tokyo Chemical Industry Co., Ltd.) was added thereto, and the mixture was stirred at room temperature for 4 hours. Thereafter, the reaction solution was gradually transferred to a separatory funnel containing 25 mL of saturated aqueous sodium bicarbonate, and then extracted twice with 50 mL of ethyl acetate. The organic layer was washed with 25 mL of saline, 25 mL of saturated aqueous sodium bicarbonate, and 25 mL of saline in this order, and then dewatered with anhydrous sodium sulfate. The drying agent was separated by filtration, and the filtrate was concentrated, and the residues were purified by silica gel column chromatography, thereby obtaining 3.58 g of a compound (AA) (two Rf₁'s in Formula (AA) are PFPE chains represented by Formula (5-1), and in two Rf₁'s, h representing the average degree of polymerization represents 4.5, and i representing the average degree of polymerization represents 4.5.).

The structure of the obtained compound (AA) was identified by performing ¹H-NMR measurement and ¹⁹F-NMR measurement based on the following results.

¹H-NMR (acetone-D₆): δ [ppm]=1.65-1.85 (4H), 3.40-3.85 (32H), 3.85-4.10 (8H)

¹⁹F-NMR (acetone-D₆): δ [ppm]=−55.5 to −51.5 (18F), −78.5 (4F), −80.5 (4F), −91.0 to −88.5 (36F)

### Example 2

A compound represented by Formula (AB) was obtained by the following method.

3.61 g of a compound (AB) (two Rf₁'s in Formula (AB) are PFPE chains represented by Formula (5-1), and in two Rf₁'s, h representing the average degree of polymerization represents 4.5, and i representing the average degree of polymerization represents 4.5) was obtained by performing the same operation as in Example 1 except that a compound (first raw material compound) represented by Formula (6-2) was used instead of the compound represented by Formula (6-1).

The compound represented by Formula (6-2) was synthesized by protecting a 1,2-diol moiety of 1,2,6-hexanetriol with acetone and then reacting a hydroxy group at the 6-position with epibromohydrin.

The structure of the obtained compound (AB) was identified by performing ¹H-NMR measurement and ¹⁹F-NMR measurement based on the following results.

¹H-NMR (acetone-D₆): δ [ppm]=1.40-1.85 (12H), 3.40-3.85 (32H), 3.85-4.10 (8H)

¹⁹F-NMR (acetone-D₆): δ [ppm]=−55.5 to −51.5 (18F), −78.5 (4F), −80.5 (4F), −91.0 to −88.5 (36F)

### Example 3

A compound represented by Formula (AC) was obtained by the following method.

3.53 g of a compound (AC) (two Rf₁'s in Formula (AB) are PFPE chains represented by Formula (5-1), and in two Rf₁'s, h representing the average degree of polymerization represents 4.5, and i representing the average degree of polymerization represents 4.5) was obtained by performing the same operation as in Example 1 except that a compound (first raw material compound) represented by Formula (6-4) was used instead of the compound represented by Formula (6-1).

The compound represented by Formula (6-4) was produced by the following method. That is, a 1,2-diol moiety of 1,2,4-butanetriol was protected with acetone. Thereafter, a hydroxy group at the 4-position was brominated and reacted with 3-butene-1-ol. The vinyl group of the compound obtained above was oxidized using m-chloroperbenzoic acid (mCPBA). The compound represented by Formula (6-4) was synthesized by performing the above-described step.

The structure of the obtained compound (AC) was identified by performing ¹H-NMR measurement and ¹⁹F-NMR measurement based on the following results.

¹H-NMR (acetone-D₆): δ [ppm]=1.65-1.85 (8H), 3.40-3.85 (32H), 3.85-4.10 (8H)

¹⁹F-NMR (acetone-D₆): δ [ppm]=−55.5 to −51.5 (18F), −78.5 (4F), −80.5 (4F), −91.0 to −88.5 (36F)

### Example 4

A compound represented by Formula (AD) was obtained by the following method.

3.67 g of a compound (AD) (two Rf₁'s in Formula (AD) are PFPE chains represented by Formula (5-1), and in the two Rf₁'s, h representing the average degree of polymerization represents 4.5, and i representing the average degree of polymerization represents 4.5) was obtained by performing the same operation as in Example 1 except that a compound (first raw material compound) represented by Formula (6-5) was used instead of the compound represented by Formula (6-1).

The compound represented by Formula (6-5) was produced by the method represented by Formula (7-2). That is, the compound was synthesized by reacting 3-butene-1-ol with a halogen compound obtained by brominating a hydroxy group of solketal (2,2-dimethyl-1,3-dioxolane-4-methanol) and oxidizing a vinyl group of the obtained compound using mCPBA.

The structure of the obtained compound (AD) was identified by performing ¹H-NMR measurement and ¹⁹F-NMR measurement based on the following results.

¹H-NMR (acetone-D₆): δ [ppm]=1.65-1.85 (4H), 3.40-3.85 (32H), 3.85-4.10 (8H)

¹⁹F-NMR (acetone-D₆): δ [ppm]=−55.5 to −51.5 (18F), −78.5 (4F), −80.5 (4F), −91.0 to −88.5 (36F)

### Example 5

A compound represented by Formula (AE) was obtained by the following method.

3.52 g of a compound (AE) (two Rf₁'s in Formula (AE) are PFPE chains represented by Formula (5-1), and in the two Rf₁'s, h representing the average degree of polymerization represents 4.5, and i representing the average degree of polymerization represents 4.5) was obtained by performing the same operation as in Example 1 except that a compound (first raw material compound) represented by Formula (6-6) was used instead of the compound represented by Formula (6-1).

The compound represented by Formula (6-6) was synthesized by reacting 5-hexene-1-ol with a halogen compound obtained by brominating a hydroxy group of solketal and oxidizing a vinyl group of the obtained compound using mCPBA.

The structure of the obtained compound (AE) was identified by performing ¹H-NMR measurement and ¹⁹F-NMR measurement based on the following results.

¹H-NMR (acetone-D₆): δ [ppm]=1.40-1.85 (12H), 3.40-3.85 (32H), 3.85-4.10 (8H)

¹⁹F-NMR (acetone-D₆): δ [ppm]=−55.5 to −51.5 (18F), −78.5 (4F), −80.5 (4F), −91.0 to −88.5 (36F)

### Example 6

A compound represented by Formula (AF) was obtained by the following method.

3.43 g of a compound (AF) (two Rf₁'s in Formula (AF) are PFPE chains represented by Formula (5-1), and in the two Rf₁'s, h representing the average degree of polymerization represents 4.5, and i representing the average degree of polymerization represents 4.5) was obtained by performing the same operation as in Example 1 except that a compound (first raw material compound) represented by Formula (6-8) was used instead of the compound represented by Formula (6-1).

The compound represented by Formula (6-8) was produced by the following method. That is, an epoxy group of 1,2-epoxy-5-hexene was ring-opened using dilute sulfuric acid. A 1,2-diol moiety of the generated compound was protected using acetone. Thereafter, the compound was synthesized by oxidizing a vinyl group of the obtained compound using mCPBA.

The structure of the obtained compound (AF) was identified by performing ¹H-NMR measurement and ¹⁹F-NMR measurement based on the following results.

¹H-NMR (acetone-D₆): δ [ppm]=1.65-1.85 (8H), 3.40-3.85 (24H), 3.85-4.10 (8H)

¹⁹F-NMR (acetone-D₆): δ [ppm]=−55.5 to −51.5 (18F), −78.5 (4F), −80.5 (4F), −91.0 to −88.5 (36F)

### Example 7

A compound represented by Formula (AG) was obtained by the following method.

3.15 g of a compound (AG) (two Rf₁'s in Formula (AG) are PFPE chains represented by Formula (5-1), and in the two Rf₁'s, h representing the average degree of polymerization represents 4.5, and i representing the average degree of polymerization represents 4.5) was obtained by performing the same operation as in Example 1 except that a compound (first raw material compound) represented by Formula (6-9) was used instead of the compound represented by Formula (6-1).

The compound represented by Formula (6-9) was produced by the following method. That is, one vinyl group of 1,7-octadiene was oxidized using mCPBA. The epoxy group generated in the above-described manner was ring-opened using dilute sulfuric acid. A 1,2-diol moiety of the generated compound was protected using acetone. Thereafter, the compound was synthesized by oxidizing a vinyl group of the obtained compound using mCPBA.

The structure of the obtained compound (AG) was identified by performing ¹H-NMR measurement and ¹⁹F-NMR measurement based on the following results.

¹H-NMR (acetone-D₆): δ [ppm]=1.40-1.85 (16H), 3.40-3.85 (24H), 3.85-4.10 (8H)

¹⁹F-NMR (acetone-D₆): δ [ppm]=−55.5 to −51.5 (18F), −78.5 (4F), −80.5 (4F), −91.0 to −88.5 (36F)

### Example 8

A compound represented by Formula (AH) was obtained by the following method.

3.91 g of a compound (AH) (two Rf₁'s in Formula (AH) are PFPE chains represented by Formula (5-1), and in the two Rf₁'s, h representing the average degree of polymerization represents 4.5, and i representing the average degree of polymerization represents 4.5) was obtained by performing the same operation as in Example 1 except that a compound (first raw material compound) represented by Formula (6-10) was used instead of the compound represented by Formula (6-1).

The compound represented by Formula (6-10) was produced by the method represented by Formula (7-3). That is, one hydroxy group of 1,3-propanediol reacted with a halogen compound obtained by brominating a hydroxy group of solketal. Thereafter, the compound was synthesized by reacting a hydroxy group of the generated alcohol compound with epibromohydrin represented by Formula (8-1).

The structure of the obtained compound (AH) was identified by performing ¹H-NMR measurement and ¹⁹F-NMR measurement based on the following results.

¹H-NMR (acetone-D₆): δ [ppm]=1.65-1.85 (4H), 3.40-3.85 (40H), 3.85-4.10 (8H)

¹⁹F-NMR (acetone-D₆): δ [ppm]=−55.5 to −51.5 (18F), −78.5 (4F), −80.5 (4F), −91.0 to −88.5 (36F)

### Example 9

A compound represented by Formula (AI) was obtained by the following method.

3.72 g of a compound (AI) (two Rf₁'s in Formula (AI) are PFPE chains represented by Formula (5-1), and in the two Rf₁'s, h representing the average degree of polymerization represents 4.5, and i representing the average degree of polymerization represents 4.5) was obtained by performing the same operation as in Example 1 except that a compound (first raw material compound) represented by Formula (6-11) was used instead of the compound represented by Formula (6-1).

The compound represented by Formula (6-11) was produced by the following method. That is, one hydroxy group of 2,2-dimethyl-1,3-propanediol reacted with a halogen compound obtained by brominating a hydroxy group of solketal. Thereafter, the compound was synthesized by reacting a hydroxy group of the generated alcohol compound with epibromohydrin.

The structure of the obtained compound (AI) was identified by performing ¹H-NMR measurement and ¹⁹F-NMR measurement based on the following results.

¹H-NMR (acetone-D₆): δ [ppm]=1.20-1.25 (12H), 3.40-3.85 (40H), 3.85-4.10 (8H)

¹⁹F-NMR (acetone-D₆): δ [ppm]=−55.5 to −51.5 (18F), −78.5 (4F), −80.5 (4F), −91.0 to −88.5 (36F)

### Example 10

A compound represented by Formula (AJ) was obtained by the following method.

3.43 g of a compound (AJ) (two Rf₁'s in Formula (AJ) are PFPE chains represented by Formula (5-1), and in the two Rf₁'s, h representing the average degree of polymerization represents 4.5, and i representing the average degree of polymerization represents 4.5) was obtained by performing the same operation as in Example 1 except that a compound (first raw material compound) represented by Formula (6-12) was used instead of the compound represented by Formula (6-1).

The compound represented by Formula (6-12) was produced by the following method. That is, one hydroxy group of 1,4-butanediol reacted with a halogen compound obtained by brominating a hydroxy group of solketal. Thereafter, the compound was synthesized by reacting a hydroxy group of the generated alcohol compound with epibromohydrin.

The structure of the obtained compound (AJ) was identified by performing ¹H-NMR measurement and ¹⁹F-NMR measurement based on the following results.

¹H-NMR (acetone-D₆): δ [ppm]=1.65-1.85 (8H), 3.40-3.85 (40H), 3.85-4.10 (8H)

¹⁹F-NMR (acetone-D₆): δ [ppm]=−55.5 to −51.5 (18F), −78.5 (4F), −80.5 (4F), −91.0 to −88.5 (36F)

### Example 11

A compound represented by Formula (AK) was obtained by the following method.

3.61 g of a compound (AK) (two Rf₁'s in Formula (AK) are PFPE chains represented by Formula (5-1), and in the two Rf₁'s, h representing the average degree of polymerization represents 4.5, and i representing the average degree of polymerization represents 4.5) was obtained by performing the same operation as in Example 1 except that a compound (first raw material compound) represented by Formula (6-13) was used instead of the compound represented by Formula (6-1).

The compound represented by Formula (6-13) was produced by the following method. That is, one hydroxy group of 2,3-dimethyl-1,4-butanediol reacted with a halogen compound obtained by brominating a hydroxy group of solketal. Thereafter, the compound was synthesized by reacting a hydroxy group of the generated alcohol compound with epibromohydrin.

The structure of the obtained compound (AK) was identified by performing ¹H-NMR measurement and ¹⁹F-NMR measurement based on the following results.

¹H-NMR (acetone-D₆): δ [ppm]=1.20-1.25 (12H), 1.65-1.85 (4H), 3.40-3.85 (40H), 3.85-4.10 (8H)

¹⁹F-NMR (acetone-D₆): δ [ppm]=−55.5 to −51.5 (18F), −78.5 (4F), −80.5 (4F), −91.0 to −88.5 (36F)

### Example 12

A compound represented by Formula (AL) was obtained by the following method.

4.21 g of a compound (AL) (two Rf₁'s in Formula (AL) are PFPE chains represented by Formula (5-1), and in the two Rf₁'s, h representing the average degree of polymerization represents 4.5, and i representing the average degree of polymerization represents 4.5) was obtained by performing the same operation as in Example 1 except that a compound (first raw material compound) represented by Formula (6-15) was used instead of the compound represented by Formula (6-1).

The compound represented by Formula (6-15) was produced by the following method. That is, one hydroxy group of 2,2,3,3-tetrafluoro-1,4-butanediol reacted with a halogen compound obtained by brominating a hydroxy group of solketal. Thereafter, the compound was synthesized by reacting a hydroxy group of the generated alcohol compound with epibromohydrin.

The structure of the obtained compound (AL) was identified by performing ¹H-NMR measurement and ¹⁹F-NMR measurement based on the following results.

¹H-NMR (acetone-D₆): δ [ppm]=3.40-3.85 (32H), 3.85-4.10 (16H)

¹⁹F-NMR (acetone-D₆): δ [ppm]=−55.5 to −51.5 (18F), −78.5 (4F), −80.5 (4F), −91.0 to −88.5 (36F), −128.5 to 130.0 (8F)

### Example 13

A compound represented by Formula (AM) was obtained by the following method.

A 100 mL eggplant flask was charged with 15 g of a compound represented by HOCH₂CF₂O(CF₂CF₂O)ₕ(CF₂O)ᵢCF₂CH₂OH (h representing the average degree of polymerization in the formula represents 4.5, and i representing the average degree of polymerization represents 4.5) (number-average molecular weight: 1,000, molecular weight distribution: 1.1), 4.50 g of 1-bromo-4-pentene, and 30 mL of N,N-dimethylformamide in a nitrogen gas atmosphere, and the mixture was stirred at room temperature until the mixture was uniform. 1.20 g of sodium hydride (purity: 60%, containing mineral oil) was added to the mixture, and the mixture was stirred at 70° C. for 16 hours to carry out a reaction.

The reaction product obtained after the reaction was cooled to 25° C., transferred to a separatory funnel charged with 50 mL of water, and extracted three times with 100 mL of ethyl acetate. The organic layer was washed with water and dewatered with anhydrous sodium sulfate. The drying agent was separated by filtration, and the filtrate was concentrated, and the residues were purified by silica gel column chromatography, thereby obtaining 6.81 g of a compound represented by Formula (10-2) as an intermediate compound 1c.

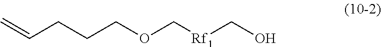

(Rf₁ in Formula (10-2) is a PFPE chain represented by Formula (5-1). In Rf₁, h representing the average degree of polymerization represents 4.5, and i representing the average degree of polymerization represents 4.5.)

Next, a 100 mL eggplant flask was charged with 6.81 g of the compound represented by Formula (10-2), which was an intermediate compound 1c obtained above, 0.58 g of epibromohydrin, and 10 mL of t-butanol in a nitrogen gas atmosphere, and the mixture was stirred at room temperature until the mixture was uniform. 0.76 g of potassium tert-butoxide was added to the uniform liquid, and the mixture was stirred at 70° C. for 23 hours to carry out a reaction.

The reaction product obtained after the reaction was cooled to 25° C., transferred to a separatory funnel charged with 50 mL of water, and extracted three times with 100 mL of ethyl acetate. The organic layer was washed with water and dewatered with anhydrous sodium sulfate. The drying agent was separated by filtration, and the filtrate was concentrated, and the residues were purified by silica gel column chromatography, thereby obtaining 4.85 g of a compound represented by Formula (10-3) as an intermediate compound.

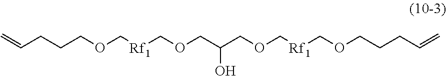

(Two Rf₁'s in Formula (10-3) are PFPE chains represented by Formula (5-1). In the two Rf₁'s, h representing the average degree of polymerization represents 4.5, and i representing the average degree of polymerization represents 4.5.)

Subsequently, a 100 mL eggplant flask was charged with 4.85 g of the compound represented by Formula (10-3), which was an intermediate compound obtained above, 20 mL of methylene chloride, and 2.45 g of mCPBA (purity: 73%, containing water) in a nitrogen gas atmosphere, and the mixture was stirred at room temperature for 16 hours to carry out a reaction.

50 mL of an aqueous solution of 2.50 g of sodium sulfite was added to the reaction solution obtained after the reaction to deactivate excess mCPBA. The generated solid was separated by filtration, transferred to a separatory funnel, and extracted three times with 100 mL of methylene chloride. The organic layer was washed with water and dewatered with anhydrous sodium sulfate. The drying agent was separated by filtration, and the filtrate was concentrated, and the residues were purified by silica gel column chromatography, thereby obtaining 4.14 g of a compound represented by Formula (10-4) as an intermediate compound.

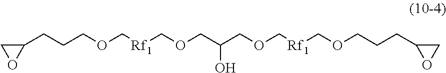

(Two Rf₁'s in Formula (10-4) are PFPE chains represented by Formula (5-1). In the two Rf₁'s, h representing the average degree of polymerization represents 4.5, and i representing the average degree of polymerization represents 4.5.)

Subsequently, a 100 mL eggplant flask was charged with 4.14 g of the compound represented by Formula (10-4), which was an intermediate compound obtained above, 20 mL of acetone, and 20 mL of 10% dilute sulfuric acid in a nitrogen gas atmosphere, and the mixture was stirred at room temperature for 16 hours to carry out a reaction.

20 mL of saturated aqueous sodium bicarbonate was added to the reaction solution obtained after the reaction to neutralize the reaction solution, and the generated solid was separated by filtration. Thereafter, the mixture was transferred to a separatory funnel and extracted three times with 100 mL of ethyl acetate. The organic layer was washed with water and dewatered with anhydrous sodium sulfate. The drying agent was separated by filtration, and the filtrate was concentrated, and the residues were purified by silica gel column chromatography, thereby obtaining 3.63 g of a compound (AM) (two Rf₁'s in Formula (AM) are PFPE chains represented by Formula (5-1), and in the two Rf₁'s, h representing the average degree of polymerization represents 4.5, and i representing the average degree of polymerization represents 4.5).

The structure of the obtained compound (AM) was identified by performing ¹H-NMR measurement and ¹⁹F-NMR measurement based on the following results.

¹H-NMR (acetone-D₆): δ [ppm]=1.65-1.85 (8H), 3.40-3.85 (20H), 3.85-4.10 (8H)

¹⁹F-NMR (acetone-D₆): δ [ppm]=−55.5 to −51.5 (18F), −78.5 (4F), −80.5 (4F), −91.0 to −88.5 (36F)

### Example 14

A compound represented by Formula (AN) was obtained by the following method.

### (First Reaction)

7.25 g of a compound represented by Formula (10-1) was obtained as an intermediate compound 1a by performing the same operation as in the first reaction of Example 1.

### (Second Reaction)

7.19 g of a compound represented by Formula (10-5) was obtained as an intermediate compound 1b by performing the same operation as in the first reaction of Example 1 except that a compound (first raw material compound) represented by Formula (6-5) was used instead of the compound represented by Formula (6-1).

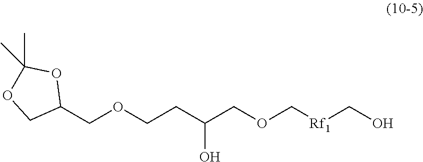

(Rf₁ in Formula (10-5) is a PFPE chain represented by Formula (5-1). In Rf₁, h representing the average degree of polymerization represents 4.5, and i representing the average degree of polymerization represents 4.5.)

Subsequently, a 100 mL eggplant flask was charged with 7.25 g of the compound represented by Formula (10-1) as the intermediate compound 1a obtained above, 4.50 g of epibromohydrin (second raw material compound) represented by Formula (8-1), and 30 mL of N,N-dimethylformamide in a nitrogen gas atmosphere, and the mixture was stirred at room temperature until the mixture was uniform. 0.25 g of sodium hydride (purity: 60%, containing mineral oil) was added to the mixture, and the mixture was stirred at room temperature for 16 hours to carry out a reaction.

The reaction product obtained after the reaction was cooled to 25° C., transferred to a separatory funnel charged with 50 mL of water, and extracted three times with 100 mL of ethyl acetate. The organic layer was washed with water and dewatered with anhydrous sodium sulfate. The drying agent was separated by filtration, and the filtrate was concentrated, and the residues were purified by silica gel column chromatography, thereby obtaining 4.81 g of a compound represented by Formula (10-6) as an intermediate compound 2a.

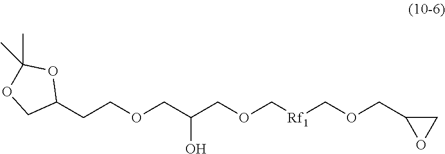

(Rf₁ in Formula (10-6) is a PFPE chain represented by Formula (5-1). In Rf₁, h representing the average degree of polymerization represents 4.5, and i representing the average degree of polymerization represents 4.5.)

Subsequently, a 100 mL eggplant flask was charged with 4.81 g of the compound represented by Formula (10-6) as the intermediate compound 2a obtained above, 7.19 g of the compound represented by Formula (10-5) as the intermediate compound 1b, and 20 mL of t-butanol in a nitrogen gas atmosphere, and the mixture was stirred at room temperature until the mixture was uniform. 0.61 g of potassium tert-butoxide was added to the mixture, and the mixture was stirred at 70° C. for 16 hours to react.

The reaction solution obtained after the reaction was cooled to room temperature, 50 g of a 10% hydrogen chloride/methanol solution (hydrogen chloride-methanol reagent (5-10%), manufactured by Tokyo Chemical Industry Co., Ltd.) was added thereto, and the mixture was stirred at room temperature for 4 hours. Thereafter, the reaction solution was gradually transferred to a separatory funnel containing 100 mL of saturated aqueous sodium bicarbonate, and then extracted twice with 200 mL of ethyl acetate. The organic layer was washed with 100 mL of saline, 100 mL of saturated aqueous sodium bicarbonate, and 100 mL of saline in this order, and then dewatered with anhydrous sodium sulfate. The drying agent was separated by filtration, and the filtrate was concentrated, and the residues were purified by silica gel column chromatography, thereby obtaining 4.21 g of a compound (AN) (Rf₁ in Formula (AN) is a PFPE chain represented by Formula (5-1). In Rf₁, h representing the average degree of polymerization represents 4.5, and i representing the average degree of polymerization represents 4.5.)

The structure of the obtained compound (AN) was identified by performing ¹H-NMR measurement and ¹⁹F-NMR measurement based on the following results.

¹H-NMR (acetone-D₆): δ [ppm]=1.65-1.85 (4H), 3.40-3.85 (32H), 3.85-4.10 (8H)

¹⁹F-NMR (acetone-D₆): δ [ppm]=−55.5 to −51.5 (18F), −78.5 (4F), −80.5 (4F), −91.0 to −88.5 (36F)

### Example 15

A compound represented by Formula (AO) was obtained by the following method.

3.51 g of a compound (AO) (two Rf₁'s in Formula (AO) are PFPE chains represented by Formula (5-1), and in the two Rf₁'s, h representing the average degree of polymerization represents 4.5, and i representing the average degree of polymerization represents 4.5) was obtained by performing the same operation as in Example 14 except that a compound (third raw material compound) represented by Formula (6-17) was used instead of the compound represented by Formula (6-5).

The compound represented by Formula (6-17) was synthesized by protecting a hydroxy group of ethylene glycol monoallyl ether using dihydropyran and then oxidizing the vinyl group with mCPBA.

The structure of the obtained compound (AO) was identified by performing ¹H-NMR measurement and ¹⁹F-NMR measurement based on the following results.

¹H-NMR (acetone-D₆): δ [ppm]=1.65-1.85 (2H), 3.40-3.85 (30H), 3.85-4.10 (8H)

¹⁹F-NMR (acetone-D₆): δ [ppm]=−55.5 to −51.5 (18F), −78.5 (4F), −80.5 (4F), −91.0 to −88.5 (36F)

### Example 16

A compound represented by Formula (AP) was obtained by the following method.

3.66 g of a compound (AP) (two Rf₁'s in Formula (AP) are PFPE chains represented by Formula (5-1), and in the two Rf₁'s, h representing the average degree of polymerization represents 4.5, and i representing the average degree of polymerization represents 4.5) was obtained by performing the same operation as in Example 14 except that a compound (third raw material compound) represented by Formula (6-18) was used instead of the compound represented by Formula (6-5).

The compound represented by Formula (6-18) was produced by the following method. That is, the epichlorohydrin and 3-butene-1-ol in an amount of twice the molar amount of the epichlorohydrin were reacted with each other. Thereafter, a hydroxy group generated by this reaction was protected using dihydropyran, and one vinyl group was oxidized with mCPBA to synthesize the compound.

The structure of the obtained compound (AP) was identified by performing ¹H-NMR measurement and ¹⁹F-NMR measurement based on the following results.

¹H-NMR (acetone-D₆): δ [ppm]=1.65-1.85 (4H), 3.40-3.85 (33H), 3.85-4.10 (8H), 5.20-5.80 (3H)

¹⁹F-NMR (acetone-D₆): δ [ppm]=−55.5 to −51.5 (18F), −78.5 (4F), −80.5 (4F), −91.0 to −88.5 (36F)

### Example 17

A compound represented by Formula (AQ) was obtained by the following method.

3.42 g of a compound (AQ) (two Rf₁'s in Formula (AQ) are PFPE chains represented by Formula (5-1), and in the two Rf₁'s, h representing the average degree of polymerization represents 4.5, and i representing the average degree of polymerization represents 4.5) was obtained by performing the same operation as in Example 14 except that a compound (third raw material compound) represented by Formula (6-19) was used instead of the compound represented by Formula (6-5).

The compound represented by Formula (6-19) was synthesized by reacting a hydroxy group of 2-acetoamidoethanol with epibromohydrin.

The structure of the obtained compound (AQ) was identified by performing H-NMR measurement and ¹⁹F-NMR measurement based on the following results.

¹H-NMR (acetone-D₆): δ [ppm]=1.65-1.85 (2H), 1.90 (3H), 3.40-3.85 (29H), 3.85-4.10 (8H), 6.70-6.80 (1H)

¹⁹F-NMR (acetone-D₆): δ [ppm]=−55.5 to −51.5 (18F), −78.5 (4F), −80.5 (4F), −91.0 to −88.5 (36F)

### Example 18

A compound represented by Formula (AR) was obtained by the following method.

3.78 g of a compound (AR) (two Rf₁'s in Formula (AR) are PFPE chains represented by Formula (5-1), and in the two Rf₁'s, h representing the average degree of polymerization represents 4.5, and i representing the average degree of polymerization represents 4.5) was obtained by performing the same operation as in Example 14 except that a compound (third raw material compound) represented by Formula (6-20) was used instead of the compound represented by Formula (6-5).

The compound represented by Formula (6-20) was synthesized by reacting a hydroxy group of 3-cyanopropanol with epibromohydrin.

The structure of the obtained compound (AR) was identified by performing ¹H-NMR measurement and ¹⁹F-NMR measurement based on the following results.

¹H-NMR (acetone-D₆): δ [ppm]=1.65-1.85 (4H), 1.90-2.00 (2H), 3.40-3.85 (27H), 3.85-4.10 (8H)

¹⁹F-NMR (acetone-D₆): δ [ppm]=−55.5 to −51.5 (18F), −78.5 (4F), −80.5 (4F), −91.0 to −88.5 (36F)

### Example 19

A compound represented by Formula (AS) was obtained by the following method.

3.44 g of a compound (AS) (two Rf₁'s in Formula (AS) are PFPE chains represented by Formula (5-1), and in the two Rf₁'s, h representing the average degree of polymerization represents 4.5, and i representing the average degree of polymerization represents 4.5) was obtained by performing the same operation as in Example 14 except that a compound (third raw material compound) represented by Formula (6-21) was used instead of the compound represented by Formula (6-5).

The compound represented by Formula (6-21) was synthesized by protecting a hydroxy group of 3-butene-1-ol using dihydropyran and then oxidizing the vinyl group with mCPBA.

The structure of the obtained compound (AS) was identified by performing ¹H-NMR measurement and ¹⁹F-NMR measurement based on the following results.

¹H-NMR (acetone-D₆): δ [ppm]=1.65-1.85 (4H), 3.40-3.85 (26H), 3.85-4.10 (8H)

¹⁹F-NMR (acetone-D₆): δ [ppm]=−55.5 to −51.5 (18F), −78.5 (4F), −80.5 (4F), −91.0 to −88.5 (36F)

### Example 20

A compound represented by Formula (AT) was obtained by the following method.

3.32 g of a compound (AT) (two Rf₁'s in Formula (AT) are PFPE chains represented by Formula (5-1), and in the two Rf₁'s, h representing the average degree of polymerization represents 4.5, and i representing the average degree of polymerization represents 4.5) was obtained by performing the same operation as in Example 14 except that a compound (third raw material compound) represented by Formula (6-22) was used instead of the compound represented by Formula (6-5).

The compound represented by Formula (6-22) was synthesized by protecting two hydroxy groups of 3-allyloxy-1,2-propanediol with dihydropyran and then oxidizing the vinyl group with mCPBA.

The structure of the obtained compound (AT) was identified by performing ¹H-NMR measurement and ¹⁹F-NMR measurement based on the following results.

¹H-NMR (acetone-D₆): δ [ppm]=1.65-1.85 (2H), 3.40-3.85 (32H), 3.85-4.10 (8H)

¹⁹F-NMR (acetone-D₆): δ [ppm]=−55.5 to −51.5 (18F), −78.5 (4F), −80.5 (4F), −91.0 to −88.5 (36F)

### Example 21

A compound represented by Formula (BA) was obtained by the following method.

3.21 g of a compound (BA) (two Rf₁'s in Formula (BA) are PFPE chains represented by Formula (5-1), and in the two Rf₁'s, h representing the average degree of polymerization represents 4.5, and i representing the average degree of polymerization represents 4.5) was obtained by performing the same operation as in Example 1 except that a compound (second raw material compound) represented by Formula (8-2) was used instead of the epibromohydrin.

The structure of the obtained compound (BA) was identified by performing ¹H-NMR measurement and ¹⁹F-NMR measurement based on the following results.

¹H-NMR (acetone-D₆): δ [ppm]=1.65-1.85 (611), 3.40-3.85 (32H), 3.85-4.10 (8H)

¹⁹F-NMR (acetone-D₆): δ [ppm]=−55.5 to −51.5 (18F), −78.5 (4F), −80.5 (4F), −91.0 to −88.5 (36F)

### Example 22

A compound represented by Formula (BB) was obtained by the following method.

2.96 g of a compound (BB) (two Rf₁'s in Formula (BB) are PFPE chains represented by Formula (5-1), and in the two Rf₁'s, h representing the average degree of polymerization represents 4.5, and i representing the average degree of polymerization represents 4.5) was obtained by performing the same operation as in Example 1 except that a compound (second raw material compound) represented by Formula (8-3) was used instead of the epibromohydrin.

The structure of the obtained compound (BB) was identified by performing ¹H-NMR measurement and ¹⁹F-NMR measurement based on the following results.

¹H-NMR (acetone-D₆): δ [ppm]=1.65-1.85 (4H), 3.40-3.85 (38H), 3.85-4.10 (8H)

¹⁹F-NMR (acetone-D₆): δ [ppm]=−55.5 to −51.5 (18F), −78.5 (4F), −80.5 (4F), −91.0 to −88.5 (36F)

### Example 23

A compound represented by Formula (BC) was obtained by the following method.

3.04 g of a compound (BC) (two Rf₁'s in Formula (BC) are PFPE chains represented by Formula (5-1), and in the two Rf₁'s, h representing the average degree of polymerization represents 4.5, and i representing the average degree of polymerization represents 4.5) was obtained by performing the same operation as in Example 1 except that a compound (second raw material compound) represented by Formula (8-4) was used instead of the epibromohydrin.

The structure of the obtained compound (BC) was identified by performing ¹H-NMR measurement and ¹⁹F-NMR measurement based on the following results.

¹H-NMR (acetone-D₆): δ [ppm]=1.65-1.85 (4H), 3.40-3.85 (34H), 3.85-4.10 (8H)

¹⁹F-NMR (acetone-D₆): δ [ppm]=−55.5 to −51.5 (18F), −78.5 (4F), −80.5 (4F), −91.0 to −88.5 (36F)

### Example 24

A compound represented by Formula (BD) was obtained by the following method.

3.43 g of a compound (BD) (two Rf₁'s in Formula (BD) are PFPE chains represented by Formula (5-1), and in the two Rf₁'s, h representing the average degree of polymerization represents 4.5, and i representing the average degree of polymerization represents 4.5) was obtained by performing the same operation as in Example 1 except that a compound (second raw material compound) represented by Formula (8-5) was used instead of the epibromohydrin.

The structure of the obtained compound (BD) was identified by performing ¹H-NMR measurement and ¹⁹F-NMR measurement based on the following results.

¹H-NMR (acetone-D₆): δ [ppm]=1.65-1.85 (8H), 3.40-3.85 (34H), 3.85-4.10 (8H)

¹⁹F-NMR (acetone-D₆): δ [ppm]=−55.5 to −51.5 (18F), −78.5 (4F), −80.5 (4F), −91.0 to −88.5 (36F)

### Example 25

A compound represented by Formula (BE) was obtained by the following method.

3.61 g of a compound (BE) (two Rf₁'s in Formula (BE) are PFPE chains represented by Formula (5-1), and in the two Rf₁'s, h representing the average degree of polymerization represents 4.5, and i representing the average degree of polymerization represents 4.5) was obtained by performing the same operation as in Example 1 except that a compound (second raw material compound) represented by Formula (8-6) was used instead of the epibromohydrin.

The compound represented by Formula (8-6) was synthesized by reacting 3-butene-1-ol with epibromohydrin and then oxidizing a vinyl group with mCPBA.

The structure of the obtained compound (BE) was identified by performing ¹H-NMR measurement and ¹⁹F-NMR measurement based on the following results.

¹H-NMR (acetone-D₆): δ [ppm]=1.65-1.85 (6H), 3.40-3.85 (38H), 3.85-4.10 (8H)

¹⁹F-NMR (acetone-D₆): δ [ppm]=−55.5 to −51.5 (18F), −78.5 (4F), −80.5 (4F), −91.0 to −88.5 (36F)

### Example 26

A compound represented by Formula (BF) was obtained by the following method.

3.20 g of a compound (BF) (two Rf₁'s in Formula (BF) are PFPE chains represented by Formula (5-1), and in the two Rf₁'s, h representing the average degree of polymerization represents 4.5, and i representing the average degree of polymerization represents 4.5) was obtained by performing the same operation as in Example 1 except that a compound (second raw material compound) represented by Formula (8-7) was used instead of the epibromohydrin.

The compound represented by Formula (8-7) was produced by the method shown in Formula (9-2). That is, the compound was synthesized by reacting epibromohydrin represented by Formula (8-1) with a 2-molar amount of 3-butene-1-ol, protecting the generated hydroxy group using dihydropyran, and oxidizing both end vinyl groups with mCPBA.

The structure of the obtained compound (BF) was identified by performing ¹H-NMR measurement and ¹⁹F-NMR measurement based on the following results.

¹H-NMR (acetone-D₆): δ [ppm]=1.65-1.85 (8H), 3.40-3.85 (44H), 3.85-4.10 (8H)

¹⁹F-NMR (acetone-D₆): δ [ppm]=−55.5 to −51.5 (18F), −78.5 (4F), −80.5 (4F), −91.0 to −88.5 (36F)

### Example 27

A compound represented by Formula (BG) was obtained by the following method.

3.41 g of a compound (BG) (two Rf₁'s in Formula (BG) are PFPE chains represented by Formula (5-1), and in the two Rf₁'s, h representing the average degree of polymerization represents 4.5, and i representing the average degree of polymerization represents 4.5) was obtained by performing the same operation as in Example 1 except that a compound (second raw material compound) represented by Formula (8-8) was used instead of the epibromohydrin.

The compound represented by Formula (8-8) was produced by the method shown in Formula (9-1). That is, the compound was synthesized by reacting 1,4-butanediol with twice the molar amount of epibromohydrin represented by Formula (8-1).

The structure of the obtained compound (BG) was identified by performing ¹H-NMR measurement and ¹⁹F-NMR measurement based on the following results.

¹H-NMR (acetone-D₆): δ [ppm]=1.65-1.85 (811), 3.40-3.85 (42H), 3.85-4.10 (8H)

¹⁹F-NMR (acetone-D₆): δ [ppm]=−55.5 to −51.5 (18F), −78.5 (4F), −80.5 (4F), −91.0 to −88.5 (36F)

### Example 28

A compound represented by Formula (BH) was obtained by the following method.

3.37 g of a compound (BH) (two Rf₁'s in Formula (BH) are PFPE chains represented by Formula (5-1), and in the two Rf₁'s, h representing the average degree of polymerization represents 4.5, and i representing the average degree of polymerization represents 4.5) was obtained by performing the same operation as in Example 1 except that a compound (second raw material compound) represented by Formula (8-9) was used instead of the epibromohydrin.

The compound represented by Formula (8-9) was synthesized by reacting 2,2-dimethyl-1,3-propanediol with twice the molar amount of epibromohydrin.

The structure of the obtained compound (BH) was identified by performing ¹H-NMR measurement and ¹⁹F-NMR measurement based on the following results.

¹H-NMR (acetone-D₆): δ [ppm]=1.20-1.25 (6H), 1.65-1.85 (4H), 3.40-3.85 (42H), 3.85-4.10 (8H)

¹⁹F-NMR (acetone-D₆): δ [ppm]=−55.5 to −51.5 (18F), −78.5 (4F), −80.5 (4F), −91.0 to −88.5 (36F)

### Example 29

A compound represented by Formula (BI) was obtained by the following method.

3.58 g of a compound (BI) (two Rf₁'s in Formula (BI) are PFPE chains represented by Formula (5-1), and in the two Rf₁'s, h representing the average degree of polymerization represents 4.5, and i representing the average degree of polymerization represents 4.5) was obtained by performing the same operation as in Example 1 except that a compound (second raw material compound) represented by Formula (8-10) was used instead of the epibromohydrin.

The compound represented by Formula (8-10) was synthesized by reacting 2,3-dimethyl-1,4-butanediol with twice the molar amount of epibromohydrin.

The structure of the obtained compound (BI) was identified by performing ¹H-NMR measurement and ¹⁹F-NMR measurement based on the following results.

¹H-NMR (acetone-D₆): δ [ppm]=1.20-1.25 (6H), 1.65-1.85 (6H), 3.40-3.85 (42H), 3.85-4.10 (8H)

¹⁹F-NMR (acetone-D₆): δ [ppm]=−55.5 to −51.5 (18F), −78.5 (4F), −80.5 (4F), −91.0 to −88.5 (36F)

### Example 30

A compound represented by Formula (BJ) was obtained by the following method.

3.97 g of a compound (BJ) (two Rf₁'s in Formula (BJ) are PFPE chains represented by Formula (5-1), and in the two Rf₁'s, h representing the average degree of polymerization represents 4.5, and i representing the average degree of polymerization represents 4.5) was obtained by performing the same operation as in Example 1 except that a compound (second raw material compound) represented by Formula (8-11) was used instead of the epibromohydrin.

The compound represented by Formula (8-11) was synthesized by reacting 2,2,3,3-tetrafluoro-1,4-butanediol with twice the molar amount of epibromohydrin.

The structure of the obtained compound (BJ) was identified by performing ¹H-NMR measurement and ¹⁹F-NMR measurement based on the following results.

¹H-NMR (acetone-D₆): δ [ppm]=1.65-1.85 (4H), 3.40-3.85 (38H), 3.85-4.10 (12H)

¹⁹F-NMR (acetone-D₆): δ [ppm]=−55.5 to −51.5 (18F), −78.5 (4F), −80.5 (4F), −91.0 to −88.5 (36F), −128.5 to 130.0 (4F)

### Example 31

A compound represented by Formula (BK) was obtained by the following method.

3.48 g of a compound (BK) (two Rf₂'s in Formula (BK) are PFPE chains represented by Formula (5-2), and in the two Rf₂'s, j representing the average degree of polymerization represents 4.5) was obtained by performing the same operation as in Example 1 except that a compound represented by HOCH₂CF₂CF₂O(CF₂CF₂CF₂O)ⱼCF₂CF₂CH₂OH (j representing the average degree of polymerization in the formula represents 4.5) (number-average molecular weight: 1,000, molecular weight distribution: 1.1) was used instead of the compound represented by HOCH₂CF₂O(CF₂CF₂O)ₕ(CF₂O)ᵢCF₂CH₂OH.

The structure of the obtained compound (BK) was identified by performing ¹H-NMR measurement and ¹⁹F-NMR measurement based on the following results.

¹H-NMR (acetone-D₆): δ [ppm]=1.65-1.85 (4H), 3.40-3.85 (32H), 3.85-4.10 (4H)

¹⁹F-NMR (acetone-D₆): δ [ppm]=−84.0 to −83.0 (36F), −86.4 (8F), −124.3 (8F), −130.0 to −129.0 (18F)

### Example 32

A compound represented by Formula (BL) was obtained by the following method.

3.38 g of a compound (BL) (Rf₃ in Formula (BL) is a PFPE chain represented by Formula (5-3), and in Rf₃, k representing the average degree of polymerization represents 3.0) was obtained by performing the same operation as in Example 1 except that a compound represented by HOCH₂CF₂CF₂CF₂O(CF₂CF₂CF₂CF₂O)ₖCF₂CF₂CF₂CH₂OH (k representing the average degree of polymerization in the formula represents 3.0) (number-average molecular weight: 1,000, molecular weight distribution: 1.1) was used instead of the compound represented by HOCH₂CF₂O(CF₂CF₂O)ₕ(CF₂O)ᵢCF₂CH₂OH.

The structure of the obtained compound (BL) was identified by performing ¹H-NMR measurement and ¹⁹F-NMR measurement based on the following results.

¹H-NMR (acetone-D₆): δ [ppm]=1.65-1.85 (4H), 3.40-3.85 (32H), 3.85-4.10 (4H)

¹⁹F-NMR (acetone-D₆): δ [ppm]=−84.0 to −83.0 (32F), −122.5 (8F), −126.0 (24F), −129.0 to −128.0 (8F)

### Example 33

A compound represented by Formula (CA) was obtained by the following method.

A compound represented by Formula (10-7) was obtained as an intermediate compound 3a by reacting the compound represented by HOCH₂CF₂O(CF₂CF₂O)ₕ(CF₂O)ᵢCF₂CH₂OH (h representing the average degree of polymerization in the formula represents 4.5, and i representing the average degree of polymerization represents 4.5) (number-average molecular weight: 1,000, molecular weight distribution: 1.1) with epibromohydrin (second raw material compound).

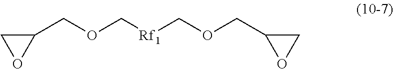

(Rf₁ in Formula (10-7) is a PFPE chain represented by Formula (5-1). In Rf₁, h representing the average degree of polymerization represents 4.5, and i representing the average degree of polymerization represents 4.5.)

Subsequently, 4.75 g of a compound (CA) (three Rf₁'s in Formula (CA) are PFPE chains represented by Formula (5-1), and in the three Rf₁'s, h representing the average degree of polymerization represents 4.5, and i representing the average degree of polymerization represents 4.5) was obtained by performing the same operation as in Example 1 except that a compound represented by Formula (10-7), which was the intermediate compound 3a, was used instead of epibromohydrin in the reaction between the compound represented by Formula (10-1) and epibromohydrin in Example 1.

The structure of the obtained compound (CA) was identified by performing ¹H-NMR measurement and ¹⁹F-NMR measurement based on the following results.

¹H-NMR (acetone-D₆): δ [ppm]=1.65-1.85 (4H), 3.40-3.85 (38H), 3.85-4.10 (12H)

¹⁹F-NMR (acetone-D₆): δ [ppm]=−55.5 to −51.5 (27F), −78.5 (6F), −80.5 (6F), −91.0 to −88.5 (54F)

### Example 34

A compound represented by Formula (CB) was obtained by the following method.

4.51 g of a compound (CB) (three Rf₁'s in Formula (CB) are PFPE chains represented by Formula (5-1), and in the three Rf₁'s, h representing the average degree of polymerization represents 4.5, and i representing the average degree of polymerization represents 4.5) was obtained by performing the same operation as in Example 33 except that a compound (first raw material compound) represented by Formula (6-5) was used instead of the compound represented by Formula (6-1) during the production of the compound represented by Formula (10-1) in Example 1.

The structure of the obtained compound (CB) was identified by performing ¹H-NMR measurement and ¹⁹F-NMR measurement based on the following results.

¹H-NMR (acetone-D₆): δ [ppm]=1.65-1.85 (4H), 3.40-3.85 (38H), 3.85-4.10 (12H)

¹⁹F-NMR (acetone-D₆): δ [ppm]=−55.5 to −51.5 (27F), −78.5 (6F), −80.5 (6F), −91.0 to −88.5 (54F)

### Example 35

A compound represented by Formula (CC) was obtained by the following method.

4.15 g of a compound (CC) (three Rf₁'s in Formula (CC) are PFPE chains represented by Formula (5-1), and in the three Rf₁'s, h representing the average degree of polymerization represents 4.5, and i representing the average degree of polymerization represents 4.5) was obtained by performing the same operation as in Example 33 except that a compound (first raw material compound) represented by Formula (6-8) was used instead of the compound represented by Formula (6-1) during the production of the compound represented by Formula (10-1) in Example 1.

The structure of the obtained compound (CC) was identified by performing ¹H-NMR measurement and ¹⁹F-NMR measurement based on the following results.

¹H-NMR (acetone-D₆): δ [ppm]=1.65-1.85 (12H), 3.40-3.85 (30H), 3.85-4.10 (12H)

¹⁹F-NMR (acetone-D₆): δ [ppm]=−55.5 to −51.5 (27F), −78.5 (6F), −80.5 (6F), −91.0 to −88.5 (54F)

### Example 36

A compound represented by Formula (CD) was obtained by the following method.

4.36 g of a compound (CD) (three Rf₁'s in Formula (CD) are PFPE chains represented by Formula (5-1), and in the three Rf₁'s, h representing the average degree of polymerization represents 4.5, and i representing the average degree of polymerization represents 4.5) was obtained by performing the same operation as in Example 33 except that a compound (first raw material compound) represented by Formula (6-10) was used instead of the compound represented by Formula (6-1) during the production of the compound represented by Formula (10-1) in Example 1.

The structure of the obtained compound (CD) was identified by performing ¹H-NMR measurement and ¹⁹F-NMR measurement based on the following results.

¹H-NMR (acetone-D₆): δ [ppm]=1.65-1.85 (8H), 3.40-3.85 (46H), 3.85-4.10 (12H)

¹⁹F-NMR (acetone-D₆): δ [ppm]=−55.5 to −51.5 (27F), −78.5 (6F), −80.5 (6F), −91.0 to −88.5 (54F)

### Example 37

A compound represented by Formula (CE) was obtained by the following method.

A compound represented by HOCH₂CF₂O(CF₂CF₂O)ₕ(CF₂O)ᵢCF₂CH₂OH (h representing the average degree of polymerization in the formula represents 4.5, and i representing the average degree of polymerization represents 4.5) (number-average molecular weight: 1,000, molecular weight distribution: 1.1) reacted with allyl glycidyl ether. A hydroxy group of a compound generated by the reaction was protected using dihydropyran, and then a double bond of the compound was oxidized with mCPBA to obtain a compound represented by Formula (10-8) as an intermediate compound 3a.

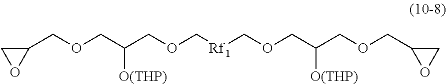

(Rf₁ in Formula (10-87) is a PFPE chain represented by Formula (5-1). In Rf₁, h representing the average degree of polymerization represents 4.5, and i representing the average degree of polymerization represents 4.5. THP represents a tetrahydropyranyl group.)

Subsequently, 4.56 g of a compound (CE) (three Rf₁'s in Formula (CE) are PFPE chains represented by Formula (5-1), and in the three Rf₁'s, h representing the average degree of polymerization represents 4.5, and i representing the average degree of polymerization represents 4.5) was obtained by performing the same operation as in Example 1 except that a compound represented by Formula (10-8), which was the intermediate compound 3a, was used instead of epibromohydrin in the reaction between the compound represented by Formula (10-1) and epibromohydrin in Example 1.

The structure of the obtained compound (CE) was identified by performing ¹H-NMR measurement and ¹⁹F-NMR measurement based on the following results.

¹H-NMR (acetone-D₆): δ [ppm]=1.65-1.85 (4H), 3.40-3.85 (50H), 3.85-4.10 (12H)

¹⁹F-NMR (acetone-D₆): δ [ppm]=−55.5 to −51.5 (27F), −78.5 (6F), −80.5 (6F), −91.0 to −88.5 (54F)

### Example 38

A compound represented by Formula (CF) was obtained by the following method.

3.87 g of a compound (CFE) (three Rf₁'s in Formula (CF) are PFPE chains represented by Formula (5-1), and in the three Rf₁'s, h representing the average degree of polymerization represents 4.5, and i representing the average degree of polymerization represents 4.5) was obtained by performing the same operation as in Example 37 except that 1,2-epoxy-5-hexene was used instead of the allylglycidyl ether.

The structure of the obtained compound (CF) was identified by performing ¹H-NMR measurement and ¹⁹F-NMR measurement based on the following results.

¹H-NMR (acetone-D₆): δ [ppm]=1.65-1.85 (12H), 3.40-3.85 (42H), 3.85-4.10 (12H)

¹⁹F-NMR (acetone-D₆): δ [ppm]=−55.5 to −51.5 (27F), −78.5 (6F), −80.5 (6F), −91.0 to −88.5 (54F)

### Example 39

A compound represented by Formula (DA) was obtained by the following method.

3.31 g of a compound (DA) (two Rf₁'s in Formula (DA) are PFPE chains represented by Formula (5-1), and in two Rf₁'s, h representing the average degree of polymerization represents 7.0 and i representing the average degree of polymerization represents 0) was obtained by performing the same operation as in Example 1 except that a compound represented by HOCH₂CF₂O(CF₂CF₂O)ₕ(CF₂O)ᵢCF₂CH₂OH (h representing the average degree of polymerization in the formula represents 7.0 and i representing the average degree of polymerization represents 0) was used instead of the compound represented by HOCH₂CF₂O(CF₂CF₂O)ₕ(CF₂O)ᵢCF₂CH₂OH (h representing the average degree of polymerization in the formula represents 4.5 and i representing the average degree of polymerization represents 4.5).

The structure of the obtained compound (DA) was identified by performing ¹H-NMR measurement and ¹⁹F-NMR measurement based on the following results.

¹H-NMR (acetone-D₆): δ [ppm]=1.65-1.85 (4H), 3.40-3.85 (32H), 3.85-4.10 (8H)

¹⁹F-NMR (acetone-D₆): δ [ppm]=−78.5 (8F), −91.0 to −88.5 (56F)

### Example 40

A compound represented by Formula (DB) was obtained by the following method.

3.81 g of a compound (DB) (two Rf₁'s in Formula (DB) are PFPE chains represented by Formula (5-1), and in two Rf₁'s, h representing the average degree of polymerization represents 7.0 and i representing the average degree of polymerization represents 0) was obtained by performing the same operation as in Example 39 except that a compound represented by Formula (6-5) was used instead of the compound represented by Formula (6-1).

The structure of the obtained compound (DB) was identified by performing ¹H-NMR measurement and ¹⁹F-NMR measurement based on the following results.

¹H-NMR (acetone-D₆): δ [ppm]=1.65-1.85 (4H), 3.40-3.85 (32H), 3.85-4.10 (8H)

¹⁹F-NMR (acetone-D₆): δ [ppm]=−78.5 (8F), −91.0 to −88.5 (56F)

### Example 41

A compound represented by Formula (DC) was obtained by the following method.

3.61 g of a compound (DC) (two Rf₂'s in Formula (DC) are PFPE chains represented by Formula (5-2), and in two Rf₂'s, j representing the average degree of polymerization represents 4.5) was obtained by performing the same operation as in Example 31 except that a compound represented by Formula (6-5) was used instead of the compound represented by Formula (6-1).

The structure of the obtained compound (DC) was identified by performing ¹H-NMR measurement and ¹⁹F-NMR measurement based on the following results.

¹H-NMR (acetone-D₆): δ [ppm]=1.65-1.85 (411), 3.40-3.85 (32H), 3.85-4.10 (4H)

¹⁹F-NMR (acetone-D₆): δ [ppm]=−84.0 to −83.0 (36F), −86.4 (8F), −124.3 (8F), −130.0 to −129.0 (18F)

### Example 42

A compound represented by Formula (DD) was obtained by the following method.

3.42 g of a compound (DD) (two Rf₂'s in Formula (DD) are PFPE chains represented by Formula (5-2), and in two Rf₂'s, j representing the average degree of polymerization represents 4.5) was obtained by performing the same operation as in Example 41 except that a compound (second raw material compound) represented by Formula (8-6) was used instead of the epibromohydrin.

The structure of the obtained compound (DD) was identified by performing ¹H-NMR measurement and ¹⁹F-NMR measurement based on the following results.

¹H-NMR (acetone-D₆): δ [ppm]=1.65-1.85 (6H), 3.40-3.85 (38H), 3.85-4.10 (8H)

¹⁹F-NMR (acetone-D₆): δ [ppm]=−84.0 to −83.0 (36F), −86.4 (8F), −124.3 (8F), −130.0 to −129.0 (18F)

### Example 43

A compound represented by Formula (DE) was obtained by the following method.

3.71 g of a compound (DE) (two Rf₂'s in Formula (DE) are PFPE chains represented by Formula (5-2), and in two Rf₂'s, j representing the average degree of polymerization represents 4.5) was obtained by performing the same operation as in Example 41 except that a compound (second raw material compound) represented by Formula (8-12) was used instead of the epibromohydrin. The compound represented by Formula (8-12) was synthesized by reacting 5-hexene-1-ol with epibromohydrin and then oxidizing a vinyl group with mCPBA.

The structure of the obtained compound (DE) was identified by performing ¹H-NMR measurement and ¹⁹F-NMR measurement based on the following results.

¹H-NMR (acetone-D₆): δ [ppm]=1.65-1.85 (10H), 3.40-3.85 (38H), 3.85-4.10 (8H)

¹⁹F-NMR (acetone-D₆): δ [ppm]=−84.0 to −83.0 (36F), −86.4 (8F), −124.3 (8F), −130.0 to −129.0 (18F)

### Example 44

A compound represented by Formula (DF) was obtained by the following method.

A 100 mL eggplant flask was charged with 12 g of a compound represented by HOCH₂CF₂CF₂O(CF₂CF₂CF₂O)ⱼCF₂CF₂CH₂OH (representing the average degree of polymerization in the formula represents 4.5) (number-average molecular weight: 1,000, molecular weight distribution: 1.1), 1.81 g of the compound represented by Formula (6-24), and 20 mL of N,N-dimethylformamide in a nitrogen gas atmosphere, and the mixture was stirred at room temperature until the mixture was uniform. 5.51 g of cesium carbonate was added to the mixture, and the mixture was stirred at 50° C. for 16 hours to carry out a reaction.

The compound represented by Formula (6-24) was synthesized by protecting a 1,2-diol moiety of 1,2,6-hexanetriol with acetone and then reacting a hydroxy group at the 6-position with p-toluenesulfonyl chloride.

The reaction product obtained after the reaction was cooled to 25° C., transferred to a separatory funnel charged with 100 mL of water, and extracted three times with 100 mL of ethyl acetate. The organic layer was washed with water and dewatered with anhydrous sodium sulfate. The drying agent was separated by filtration, and the filtrate was concentrated, and the residues were purified by silica gel column chromatography, thereby obtaining 6.04 g of a compound represented by Formula (10-9) as an intermediate compound 1.

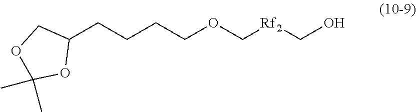

(Rf₂ in Formula (10-9) is a PFPE chain represented by Formula (5-2). In Rf₂, j representing the average degree of polymerization represents 4.5.)

Next, a 100 mL eggplant flask was charged with 6.04 g of the compound represented by Formula (10-9), which was an intermediate compound 1 obtained above, 0.45 g the compound (second raw material compound) represented by formula (8-12), and 10 mL of t-butanol in a nitrogen gas atmosphere, and the mixture was stirred at room temperature until the mixture was uniform. 0.72 g of potassium tert-butoxide was added to the uniform liquid, and the mixture was stirred at 70° C. for 23 hours to carry out a reaction.

The reaction solution obtained after the reaction was cooled to room temperature, 10 g of a 10% hydrogen chloride/methanol solution (hydrogen chloride-methanol reagent (5-10%) manufactured by Tokyo Chemical Industry Co., Ltd.) was added thereto, and the mixture was stirred at room temperature for 4 hours. Thereafter, the reaction solution was gradually transferred to a separatory funnel containing 25 mL of saturated aqueous sodium bicarbonate, and then extracted twice with 50 mL of ethyl acetate. The organic layer was washed with 25 mL of saline, 25 mL of saturated aqueous sodium bicarbonate, and 25 mL of saline in this order, and then dewatered with anhydrous sodium sulfate. The drying agent was separated by filtration, and the filtrate was concentrated, and the residues were purified by silica gel column chromatography, thereby obtaining 3.31 g of a compound (DF) (two Rf₂'s in Formula (DF) are PFPE chains represented by Formula (5-2), and in two Rf₂'s, j representing the average degree of polymerization represents 4.5).

The structure of the obtained compound (DF) was identified by performing ¹H-NMR measurement and ¹⁹F-NMR measurement based on the following results.

¹H-NMR (acetone-D₆): δ [ppm]=1.65-1.85 (18H), 3.40-3.85 (26H), 3.85-4.10 (8H)

¹⁹F-NMR (acetone-D₆): δ [ppm]=−84.0 to −83.0 (36F), −86.4 (8F), −124.3 (8F), −130.0 to −129.0 (18F)

### Example 45

A compound represented by Formula (DG) was obtained by the following method.

3.36 g of a compound (DG) (two Rf₂'s in Formula (DG) are PFPE chains represented by Formula (5-2), and in two Rf₂'s, j representing the average degree of polymerization represents 4.5) was obtained by performing the same operation as in Example 44 except that epibromohydrin represented by Formula (8-1) was used instead of the compound represented by Formula (8-12) and a compound represented by Formula (6-25) was used instead of the compound represented by Formula (6-24).

The compound represented by Formula (6-25) was synthesized by the following method. First, a hydroxy group of 5-hexene-1-ol was protected using dihydropyran, and then the alkenyl group was oxidized with m-chloroperbenzoic acid. Thereafter, the obtained compound reacted with solketal, and the secondary hydroxy group of the generated compound was protected using chloromethyl methyl ether. The obtained compound represented by Formula (6-25A) was treated with an acid to selectively deprotect the THP group, and then reacted with p-toluenesulfonyl to react with the primary hydroxy group to obtain a compound represented by Formula (6-25).

The selective deprotection of the THP group was carried out by adding 1.21 g (4.83 mmol) of p-toluenesulfonic acid pyridinium, which was an acid catalyst, to a mixture of 9.09 g (24.2 mmol) of the compound represented by Formula (6-25A) and a mixed solvent formed by mixing 40 g of 2-propanol and 40 g of acetone, and stirring the mixture in an air atmosphere at a reaction temperature of 55° C. for 7 hours.

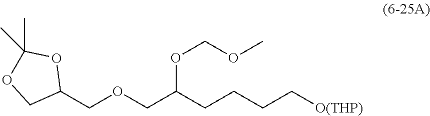

The structure of the obtained compound (DG) was identified by performing ¹H-NMR measurement and ¹⁹F-NMR measurement based on the following results.

¹H-NMR (acetone-D₆): δ [ppm]=1.65-1.85 (12H), 3.40-3.85 (32H), 3.85-4.10 (8H)

¹⁹F-NMR (acetone-D₆): δ [ppm]=−84.0 to −83.0 (36F), −86.4 (8F), −124.3 (8F), −130.0 to −129.0 (18F)

### Example 46

A compound represented by Formula (DH) was obtained by the following method.

3.21 g of a compound (DH) (three Rf₂'s in Formula (DH) are PFPE chains represented by Formula (5-2), and in the three Rf₂'s, j representing the average degree of polymerization represents 4.5) was obtained by performing the same operation as in Example 34 except that a compound represented by HOCH₂CF₂CF₂O(CF₂CF₂CF₂O)ⱼCF₂CF₂CH₂OH (j representing the average degree of polymerization in the formula represents 4.5) (number-average molecular weight: 1,000, molecular weight distribution: 1.1) was used instead of the compound represented by HOCH₂CF₂O(CF₂CF₂O)ₙ(CF₂O)ᵢCF₂CH₂OH.

The structure of the obtained compound (DH) was identified by performing ¹H-NMR measurement and ¹⁹F-NMR measurement based on the following results.

¹H-NMR (acetone-D₆): δ [ppm]=1.65-1.85 (4H), 3.40-3.85 (38H), 3.85-4.10 (12H)

¹⁹F-NMR (acetone-D₆): δ [ppm]=−84.0 to −83.0 (54F), −86.4 (12F), −124.3 (12F), −130.0 to −129.0 (27F)

The structures of x, R¹, R², R³, and R⁴ in a case where each of the compounds (AA) to (AT), (BA) to (BL), (CA) to (CF), and (DA) to (DH) of Examples 1 to 46 obtained as described above was applied to Formula (1) are listed in Tables 1 to 3.

TABLE 1

| Compound | X | R¹ | | R² | R³ | | R⁴ | |
|---|---|---|---|---|---|---|---|---|
| (AA) | 1 | (2-1) | a = 1/b = 1 | (5-1) | (4-1) | u1 = 0/u2 = 0 | (2-1) | Same as R¹ |
| (AB) | 1 | (2-1) | a = 1/b = 3 | (5-1) | (4-1) | u1 = 0/u2 = 0 | (2-1) | Same as R¹ |
| (AC) | 1 | (2-1) | a = 2/b = 1 | (5-1) | (4-1) | u1 = 0/u2 = 0 | (2-1) | Same as R¹ |
| (AD) | 1 | (2-2) | c = 1 | (5-1) | (4-1) | u1 = 0/u2 = 0 | (2-2) | Same as R¹ |
| (AE) | 1 | (2-2) | c = 3 | (5-1) | (4-1) | u1 = 0/u2 = 0 | (2-2) | Same as R¹ |
| (AF) | 1 | (2-3) | d = 2 | (5-1) | (4-1) | u1 = 0/u2 = 0 | (2-3) | Same as R¹ |
| (AG) | 1 | (2-3) | d = 4 | (5-1) | (4-1) | u1 = 0/u2 = 0 | (2-3) | Same as R¹ |
| (AH) | 1 | (2-4) | e = 1, | (5-1) | (4-1) | u1 = 0/u2 = 0 | (2-4) | Same as R¹ |
| | | | Rª = R^{b} = | | | | | |
| | | | hydrogen | | | | | |
| | | | atom | | | | | |
| (AI) | 1 | (2-4) | e = 1, | (5-1) | (4-1) | u1 = 0/u2 = 0 | (2-4) | Same as R¹ |
| | | | Rª = R^{b} = | | | | | |
| | | | methyl group | | | | | |
| (AJ) | 1 | (2-4) | e = 2, | (5-1) | (4-1) | u1 = 0/u2 = 0 | (2-4) | Same as R¹ |
| | | | Rª = R^{b} = | | | | | |
| | | | hydrogen | | | | | |
| | | | atom | | | | | |
| (AK) | 1 | (2-4) | e = 2, | (5-1) | (4-1) | u1 = 0/u2 = 0 | (2-4) | Same as R¹ |
| | | | Rª = | | | | | |
| | | | hydrogen | | | | | |
| | | | atom, | | | | | |
| | | | R^{b} = methyl | | | | | |
| | | | group | | | | | |
| (AL) | 1 | (2-5) | f = 2 | (5-1) | (4-1) | u1 = 0/u2 = 0 | (2-5) | Same as R¹ |
| (AM) | 1 | (2-6) | g = 2 | (5-1) | (4-1) | u1 = 0/u2 = 0 | (2-6) | Same as R¹ |
| (AN) | 1 | (2-1) | a = 1/b = 1 | (5-1) | (4-1) | u1 = 0/u2 = 0 | (2-2) | c = 1 |
| (AO) | 1 | (2-1) | a = 1/b = 1 | (5-1) | (4-1) | u1 = 0/u2 = 0 | (3-1) | p = 0/q = 0/r = 1/ |
| | | | | | | | | D = hydroxy |
| | | | | | | | | group |
| (AP) | 1 | (2-1) | a = 1/b = 1 | (5-1) | (4-1) | u1 = 0/u2 = 0 | (3-1) | p = 1/q = 1/r = 1/ |
| | | | | | | | | D = vinyl group |
| (AQ) | 1 | (2-1) | a = 1/b = 1 | (5-1) | (4-1) | u1 = 0/u2 = 0 | (3-1) | p = 0/q = 0/r = 1/ |
| | | | | | | | | D = acetamido |
| | | | | | | | | group |
| (AR) | 1 | (2-1) | a = 1/b = 1 | (5-1) | (4-1) | u1 = 0/u2 = 0 | (3-1) | p = 0/q = 0/r = 2/ |
| | | | | | | | | D = cyano group |
| (AS) | 1 | (2-1) | a = 1/b = 1 | (5-1) | (4-1) | u1 = 0/u2 = 0 | (3-2) | s = 0/t = 1 |
| (AT) | 1 | (2-1) | a = 1/b = 1 | (5-1) | (4-1) | u1 = 0/u2 = 0 | (3) | I = 2/m = 1/n = 1/ |
| | | | | | | | | B = hydrogen |
| | | | | | | | | atom |

TABLE 2

| Compound | X | R¹ | | R² | R³ | | R⁴ | |
|---|---|---|---|---|---|---|---|---|
| (BA) | 1 | (2-1) | a = 1/b = 1 | (5-1) | (4-1) | u1 = 0/u2 = 1 | (2-1) | Same as R¹ |
| (BB) | 1 | (2-1) | a = 1/b = 1 | (5-1) | (4-2) | v = 1 | (2-1) | Same as R¹ |
| (BC) | 1 | (2-1) | a = 1/b = 1 | (5-1) | (4-3) | w = 0 | (2-1) | Same as R¹ |
| (BD) | 1 | (2-1) | a = 1/b = 1 | (5-1) | (4-3) | w = 4 | (2-1) | Same as R¹ |
| (BE) | 1 | (2-1) | a = 1/b = 1 | (5-1) | (4-4) | x1 = 1/x2 = 0 | (2-1) | Same as R¹ |
| (BI) | 1 | (2-1) | a = 1/b = 1 | (5-1) | (4-5) | y1 = 1/y2 = 1 | (2-1) | Same as R¹ |
| (BG) | 1 | (2-1) | a = 1/b = 1 | (5-1) | (4-6) | z = 2, | (2-1) | Same as R¹ |
| | | | | | | R^{c} = R^{d} = | | |
| | | | | | | hydrogen atom | | |
| (BH) | 1 | (2-1) | a = 1/b = 1 | (5-1) | (4-6) | 7 = 1, | (2-1) | Same as R¹ |
| | | | | | | R^{c} = R^{d} = | | |
| | | | | | | methyl group | | |
| (BI) | 1 | (2-1) | a = 1/b = 1 | (5-1) | (4-6) | z = 2, | (2-1) | Same as R¹ |
| | | | | | | R^{c} = hydrogen | | |
| | | | | | | atom, | | |
| | | | | | | R^{d} = methyl group | | |
| (BJ) | 1 | (2-1) | a = 1/b = 1 | (5-1) | (4-6) | z = 2, | (2-1) | Same as R¹ |
| | | | | | | R^{c} = R^{d} = | | |
| | | | | | | fluorine atom | | |
| (BK) | 1 | (2-1) | a = 1/b = 1 | (5-2) | (4-1) | u1 = 0/u2 = 0 | (2-1) | Same as R¹ |
| (BL) | 1 | (2-1) | a = 1/b = 1 | (5-3) | (4-1) | u1 = 0/u2 = 0 | (2-1) | Same as R¹ |

TABLE 3

| Compound | X | R¹ | | R² | R³ | | R⁴ | |
|---|---|---|---|---|---|---|---|---|
| (CA) | 2 | (2-1) | a = 1/b = 1 | (5-1) | (4-1) | u1 = 0/u2 = 0 | (2-1) | Same as R¹ |
| (CB) | 2 | (2-2) | c = 1 | (5-1) | (4-1) | u1 = 0/u2 = 0 | (2-2) | Same as R¹ |
| (CC) | 2 | (2-3) | d = 2 | (5-1) | (4-1) | u1 = 0/u2 = 0 | (2-3) | Same as R¹ |
| (CD) | 2 | (2-4) | e = 1 | (5-1) | (4-1) | u1 = 0/u2 = 0 | (2-4) | Same as R¹ |
| | | | Rª = R^{b} = | | | | | |
| | | | hydrogen atom | | | | | |
| (CE) | 2 | (2-1) | a = 1/b = 1 | (5-1) | (4-2) | v = 1 | (2-1) | Same as R¹ |
| (CF) | 2 | (2-1) | a = 1/b = 1 | (5-1) | (4-3) | w = 2 | (2-1) | Same as R¹ |
| (DA) | 1 | (2-1) | a = 1/b = 1 | (5-1) | (4-1) | u1 = 0/u2 = 0 | (2-1) | a = 1/b = 1 |
| (DB) | 1 | (2-2) | c = 1 | (5-1) | (4-1) | u1 = 0/u2 = 0 | (2-2) | c = 1 |
| (DC) | 1 | (2-2) | c = 1 | (5-2) | (4-1) | u1 = 0/u2 = 0 | (2-2) | c = 1 |
| (DD) | 1 | (2-2) | c = 1 | (5-2) | (4-4) | x1 = 1/x2 = 0 | (2-2) | c = 1 |
| (DE) | 1 | (2-2) | c = 1 | (5-2) | (4-4) | x1 = 3/x2 = 0 | (2-2) | c = 1 |
| (DF) | 1 | (2-6) | g = 3 | (5-2) | (4-4) | x1 = 3/x2 = 0 | (2-6) | g = 3 |
| (DG) | 1 | (2-7) | g2 = 3 | (5-2) | (4-1) | u1 = 0/u2 = 0 | (2-7) | g2 = 3 |
| (DH) | 2 | (2-2) | c = 1 | (5-2) | (4-1) | u1 = 0/u2 = 0 | (2-2) | c = 1 |

### Comparative Example 1

A compound represented by Formula (ZA) was synthesized by the method described in Patent Document 1.

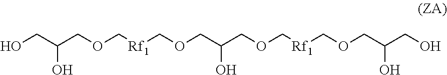

(Two Rf₁'s in Formula (ZA) are PFPE chains represented by Formula (5-1). In the two Rf₁'s, h representing the average degree of polymerization represented 7.0, and i representing the average degree of polymerization represented 0.)

### Comparative Example 2

A compound represented by Formula (ZB) was synthesized by the method described in Patent Document 2.

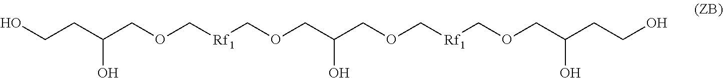

(Two Rf₁'s in Formula (ZB) are PFPE chains represented by Formula (5-1). In the two Rf₁'s, h representing the average degree of polymerization represents 4.5, and i representing the average degree of polymerization represents 4.5.)

### Comparative Example 3

A compound represented by Formula (ZC) was synthesized by the method described in Patent Document 3.

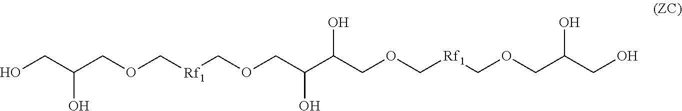

(Two Rf₁'s in Formula (ZC) are PFPE chains represented by Formula (5-1). In the two Rf₁'s, h representing the average degree of polymerization represents 4.5, and i representing the average degree of polymerization represents 4.5.)

### Comparative Example 4

A compound represented by Formula (ZD) was synthesized by the method described in Patent Document 4.

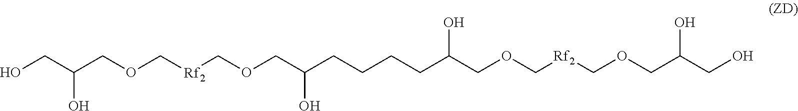

(Two Rf₂'s in Formula (ZD) are PFPE chains represented by Formula (5-2). In the two Rf₂'s, j representing the average degree of polymerization represents 4.5.)

### Comparative Example 5

A compound represented by Formula (ZE) was synthesized by the method described in Patent Document 5.

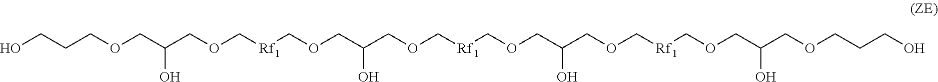

(Three Rf₁'s in Formula (ZE) are PFPE chains represented by Formula (5-1). In the three Rf₁'s, h representing the average degree of polymerization represents 4.5, and i representing the average degree of polymerization represents 4.5.)

The number-average molecular weights (Mn) of the compounds of Examples 1 to 46 and Comparative Examples 1 to 5, which were obtained as described above, were measured by the above-described method. The results are listed in Tables 4 to 6.

Next, a lubricating layer forming solution was prepared using the compounds obtained in Examples 1 to 46 and Comparative Examples 1 to 5 by the following method. Next, a lubricating layer of a magnetic recording medium was formed by the following method using the obtained lubricating layer forming solution, thereby obtaining magnetic recording media of Examples 1 to 46 and Comparative Examples 1 to 5.

### “Solution for Forming Lubricating Layer”

The fluorine-containing ether compounds obtained in Examples 1 to 46 and Comparative Examples 1 to 5 were each dissolved in VERTREL (registered trademark) XF (trade name, manufactured by DuPont Mitsui Fluorochemicals Co., Ltd.), which is a fluorine-based solvent, and diluted with VERTREL XF such that the film thickness in a case where the solution was applied onto the protective layer reached 9.0 Å to 9.5 Å, thereby preparing a lubricating layer forming solution.

### “Magnetic Recording Medium”

A magnetic recording medium in which an adhesive layer, a soft magnetic layer, a first underlayer, a second underlayer, a magnetic layer, and a protective layer were sequentially provided on a substrate having a diameter of 65 mm was prepared. The protective layer was formed of carbon.

The protective layer of the magnetic recording medium on which each layer up to the protective layer was formed was coated with each lubricating layer forming solution of Examples 1 to 46 and Comparative Examples 1 to 5 by a dipping method. Further, the dipping method was carried out under the conditions of an immersion speed of 10 mm/sec, an immersion time of 30 sec, and a pulling-up speed of 1.2 mm/sec.

Thereafter, the magnetic recording medium coated with the lubricating layer forming solution was placed in a constant temperature tank, and a thermal treatment was performed at 120° C. for 10 minutes to remove the solvent in the lubricating layer forming solution and to improve the adhesion between the protective layer and the lubricating layer so that a lubricating layer was formed on the protective layer, thereby obtaining a magnetic recording medium.

### (Measurement of Film Thickness)

The film thicknesses of the lubricating layers of the magnetic recording media of Examples 1 to 46 and Comparative Examples 1 to 5 obtained above were measured using a Fourier transform infrared spectrophotometer (FT-IR, trade name: Nicolet iS50, manufactured by Thermo Fisher Scientific). The results are listed in Tables 4 to 6.

Next, the magnetic recording media of Examples 1 to 46 and Comparative Examples 1 to 5 were subjected to the corrosion resistance test and the pickup characteristic test described below. The results are listed in Tables 4 to 6.

### [Corrosion Resistance Test]

The magnetic recording medium was exposed to an environment of a temperature of 85° C. and a relative humidity of 90% for 48 hours. Thereafter, the number of the corrosion spots having a diameter of 5 m or greater generated on the surface of the magnetic recording medium was counted using an optical surface analyzer (Candela 7140, manufactured by KLA-Tencor Corporation), and evaluated according to the following evaluation criteria.

### “Evaluation Criteria of Corrosion Resistance”

A+: The number of corrosion spots was less than 100.
A: The number of corrosion spots was 100 or more and less than 300.
B: The number of corrosion spots was 300 or more and less than 500.
C: The number of corrosion spots was 500 or more and less than 1,000.
D: The number of corrosion spots was 1,000 or more.

### [Pickup Characteristic Test]

The magnetic recording medium and the magnetic head were mounted on a spin stand, and the spin stand rotated at room temperature under reduced pressure (about 250 torr) to allow the magnetic head to float at a fixed point for 10 minutes. Thereafter, the surface (surface of the lubricating layer) of the magnetic head facing the magnetic recording medium was analyzed using an analyzer of electron spectroscopy for chemical analysis (ESCA). The amount of the lubricant adhering to the magnetic head was evaluated from the intensity (signal intensity (a.u.)) of the fluorine-derived peak obtained by the analysis using the ESCA analyzer according to the following evaluation criteria.

### “Evaluation Criteria of Pickup Characteristics”

A+: The signal intensity was less than 120 (almost no adhesion)
A: The signal intensity was 120 or greater and less than 180 (the adhesion amount was extremely small).
B: The signal intensity was 180 or greater and less than 300 (the adhesion amount was small).
C: The signal intensity was 300 or greater and less than 1000 (the adhesion amount was large).
D: The signal intensity was 1,000 or greater (the adhesion amount was extremely large).

### [Comprehensive Evaluation]

Based on the results of the corrosion resistance test and the pickup characteristic test, the comprehensive evaluation was performed according to the following criteria.

### “Comprehensive Evaluation Criteria”

A: Both the evaluation of the corrosion resistance test and the evaluation of the pickup characteristic test were A+ or A.
B: One of the evaluation of the corrosion resistance test or the evaluation of the pickup characteristic test was B, and the other was A+, A, or B.
C: One of the evaluation of the corrosion resistance test or the evaluation of the pickup characteristic test was C, and the other was A+, A, B, or C.
D: At least one of the evaluation of the corrosion resistance test or the evaluation of the pickup characteristic test was D.

TABLE 4

| | | | Film | Corrosion | Pickup | |
|---|---|---|---|---|---|---|
| | | Molecular | thickness | resistance | characteristic | Comprehensive |
| | Compound | weight | (Å) | test | test | evaluation |
| Example 1 | (AA) | 2371 | 9.4 | A | A+ | A |
| Example 2 | (AB) | 2425 | 9.4 | A | A+ | A |
| Example 3 | (AC) | 2493 | 9.3 | A | A+ | A |
| Example 4 | (AD) | 2368 | 9.1 | A | A+ | A |
| Example 5 | (AE) | 2432 | 9.2 | A | A+ | A |
| Example 6 | (AF) | 2327 | 9.4 | A+ | A | A |
| Example 7 | (AG) | 2364 | 9.3 | A+ | A | A |
| Example 8 | (AH) | 2451 | 9.3 | A | A+ | A |
| Example 9 | (AI) | 2502 | 9.4 | A | A+ | A |
| Example 10 | (AJ) | 2476 | 9.4 | A | A+ | A |
| Example 11 | (AK) | 2533 | 9.4 | A | A+ | A |
| Example 12 | (AL) | 2577 | 9.5 | A+ | A | A |
| Example 13 | (AM) | 2265 | 9.1 | A+ | B | B |
| Example 14 | (AN) | 2373 | 9.0 | A | A+ | A |
| Example 15 | (AO) | 2322 | 9.1 | A | A | A |
| Example 16 | (AP) | 2424 | 9.2 | A | A | A |
| Example 17 | (AQ) | 2359 | 9.0 | A | A | A |
| Example 18 | (AR) | 2343 | 9.4 | A | A | A |
| Example 19 | (AS) | 2296 | 9.1 | A | A | A |
| Example 20 | (AT) | 2351 | 9.5 | B | A | B |

TABLE 5

| | | | Film | Corrosion | Pickup | |
|---|---|---|---|---|---|---|
| | | Molecu1ar | thickness | resistance | characteristic | Comprehensive |
| | Compound | weight | (Å) | test | test | evaluation |
| Example 21 | (BA) | 2384 | 9.3 | A+ | A | A |
| Example 22 | (BB) | 2441 | 9.3 | B | A+ | B |
| Example 23 | (BC) | 2400 | 9.5 | A | A+ | A |
| Example 24 | (BD) | 2454 | 9.1 | A+ | B | B |
| Example 25 | (BE) | 2458 | 9.2 | B | A | B |
| Example 26 | (BF) | 2546 | 9.4 | A | A+ | A |
| Example 27 | (BG) | 2523 | 9.3 | A | A+ | A |
| Example 28 | (BH) | 2537 | 9.0 | A | A+ | A |
| Example 29 | (BI) | 2545 | 9.2 | A | A+ | A |
| Example 30 | (BJ) | 2582 | 9.4 | A | A+ | A |
| Example 31 | (BK) | 2373 | 9.3 | A | A+ | A |
| Example 32 | (BL) | 2366 | 9.3 | A | A+ | A |
| Example 33 | (CA) | 3450 | 9.3 | A | A+ | A |
| Example 34 | (CB) | 3445 | 9.2 | A | A+ | A |
| Example 35 | (CC) | 3421 | 9.4 | A+ | A | A |
| Example 36 | (CD) | 3504 | 9.3 | A | A+ | A |
| Example 37 | (CE) | 3580 | 9.4 | B | A+ | B |
| Example 38 | (CF) | 3561 | 9.1 | A+ | B | B |

TABLE 6

| | | | Film | Corrosion | Pickup | |
|---|---|---|---|---|---|---|
| | | Molecu1ar | thickness | resistance | characteristic | Comprehensive |
| | Compound | weight | (A) | test | test | evaluation |
| Example 39 | (DA) | 2365 | 9.0 | A | A+ | A |
| Example 40 | (DB) | 2378 | 9.1 | A | A+ | A |
| Example 41 | (DC) | 2381 | 9.4 | A | A+ | A |
| Example 42 | (DD) | 2463 | 9.2 | B | A | B |
| Example 43 | (DE) | 2488 | 9.3 | A | A | A |
| Example 44 | (DF) | 2543 | 9.1 | A+ | B | B |
| Example 45 | (DG) | 2602 | 9.3 | A+ | A | A |
| Example 46 | (DH) | 3461 | 9.2 | A | A+ | A |
| Comparative | (ZA) | 2206 | 9.4 | D | C | D |
| Example 1 | | | | | | |
| Comparative | (ZB) | 2230 | 9.5 | B | C | C |
| Example 2 | | | | | | |
| Comparative | (ZC) | 2231 | 9.5 | D | B | D |
| Example 3 | | | | | | |
| Comparative | (ZD) | 2383 | 9.0 | B | D | D |
| Example 4 | | | | | | |
| Comparative | (ZE) | 3442 | 9.5 | B | C | C |
| Example 5 | | | | | | |

As listed in Tables 4 to 6, in all of the magnetic recording media of Examples 1 to 46 in which the fluorine-containing ether compounds (AA) to (AT), (BA) to (BL), (CA) to (CF), and (DA) to (DH), which were represented by Formula (1) in which at least one of R¹ or R⁴ disposed at the end of the perfluoropolyether chain represents an end group represented by any of Formulae (2-1) to (2-7), were used, the evaluations of both the corrosion resistance test and the pickup characteristic test were A+, A, or B, and the comprehensive evaluation was A or B. As shown in the results, it was confirmed that the lubricating layers of the magnetic recording media of Examples 1 to 46 had satisfactory corrosion resistance and had a high pickup suppression effect.

In particular, in Examples 6, 7, 12, 13, 35, and 45 in which the compounds (AF), (AG), (AL), (AM), (CC), and (DG) in which R³ in Formula (1) represents a linking group represented by Formula (4-1), u1 and u2 represent 0, and R¹ and R⁴ represent an end group represented by any of Formulae (2-3), (2-5), (2-6), and (2-7) were used, the evaluation of the corrosion resistance test was A+, which showed a satisfactory result. As shown in the results, it was confirmed that the fluorine-containing ether compound having an end group represented by any of Formulae (2-3), (2-5), (2-6), and (2-7) could form a lubricating layer having higher corrosion resistance.

In addition, in Examples 1, 2, 3, 4, 5, 8, 9, 10, 11, 31, 32, 33, 34, 36, 39, 40, 41, and 46 in which the compounds (AA) to (AE), (AH) to (AK), (BK), (BL), (CA), (CB), (CD), (DA) to (DC), and (DH), in which R³ in Formula (1) represents a linking group represented by Formula (4-1) u1 and u2 represent 0 and R¹ and R⁴ represent an end group represented by Formulae (2-1), (2-2), and (2-4), were used, the evaluation of the pickup characteristic test was A+, which showed a satisfactory result. As shown in the results, it was confirmed that the fluorine-containing ether compound having an end group represented by any of Formulae (2-1), (2-2), and (2-4) could form a lubricating layer having more excellent pickup resistance.

In addition, in case of the compounds in which R¹ and R⁴ represent an end group represented by Formula (2-1), a represents 1, b represents 1, in Examples 21, 24, and 38 in which the compound (BA) in which R³ represents a linking group represented by Formula (4-1), u1 represents 0, and u2 represents 1, the compound (BD) in which R³ represents a linking group represented by Formula (4-3) and w represents 4, and the compound (CF) in which w represents 2 were used, the evaluation of the corrosion resistance test was A+, which showed satisfactory results.

It is presumed that the above-described results were obtained due to the formation of the lubricating layer having more excellent hydrophobicity because the linking group represented by Formulae (4-1) and (4-3) has two or less hydroxy groups, does not have an ether bond except for both ends of the linking group, and has a carbon atom that was not bonded to either the polar group or the ether oxygen atom.

Meanwhile, as listed in Table 6, in Comparative Examples 1 to 5 in which the compounds (ZA) to (ZE) were used, all the evaluations of the corrosion resistance test and the pickup characteristic test were any of B, C, or D, and the comprehensive evaluation was any of C or D.

More specifically, the compound (ZA) used in Comparative Example 1 and the compound (ZC) used in Comparative Example 3 had a 1,2-diol structure disposed at both ends, but the end group did not have a carbon atom that was not bonded to either the polar group or the ether oxygen atom. Therefore, in the lubricating layer formed of the compounds (ZA) and (ZC), it was considered that the hydrophobicity was not sufficiently obtained, water, which causes corrosion, was easily taken into the layer, and thus the result of the corrosion resistance test in Comparative Example 1 and Comparative Example 3 was D.

The compound (ZB) used in Comparative Example 2 and the compound (ZE) used in Comparative Example 5 had a carbon atom not bonded to either the polar group or the ether oxygen atom between two hydroxy groups included in each of the both end groups, but did not have a 1,2-diol structure. Therefore, in the compounds (ZB) and (ZE), all hydroxy groups were disposed by being sufficiently separated from adjacent hydroxy groups, and all hydroxy groups were considered to easily interact with the protective layer. As a result, it was considered that the intermolecular interaction of the fluorine-containing ether compound was small, the lubricating layer easily moved from the protective layer to the magnetic head in a case where the magnetic head collided with the lubricating layer, and thus the evaluation of the pickup characteristic test of Comparative Example 2 and Comparative Example 5 was C.

The compound (ZD) used in Comparative Example 4 has a 1,2-diol structure disposed at both ends, but the end group did not have a carbon atom that was not bonded to either the polar group or the ether oxygen atom. Therefore, in the compound (ZD), moderate rigidity obtained in a case where the end group had a carbon atom that was not bonded to either the polar group or the ether oxygen atom was not obtained, and two hydroxy groups constituting a 1,2-diol structure were likely to interact with each other in the molecule. As a result, in the lubricating layer formed of the compound (ZD), the hydroxy groups in the compound were unlikely to interact with each other between molecules, the lubricating layer easily moved from the protective layer to the magnetic head in a case where the magnetic head collided with the lubricating layer, and thus the evaluation result of the pickup characteristic test of Comparative Example 4 was considered to be D.

### INDUSTRIAL APPLICABILITY

By using the lubricant for a magnetic recording medium containing the fluorine-containing ether compound according to the embodiment of the present invention, it is possible to form a lubricating layer having satisfactory corrosion resistance and a high pickup suppression effect even in a case where the thickness of the layer is small.

### REFERENCE SIGNS LIST

10: magnetic recording medium
11: substrate
12: adhesive layer
13: soft magnetic layer
14: first underlayer
15: second underlayer
16: magnetic layer
17: protective layer
18: lubricating layer

1. A fluorine-containing ether compound which is represented by Formula (1), 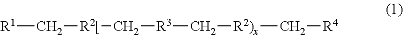
(in Formula (1), x represents an integer of 1 or 2; R² represents a perfluoropolyether chain; (x+1) pieces of R²'s may be partially or entirely the same as or different from each other; R³ represents a divalent linking group having one to four polar groups; two R³'s may be the same as or different from each other in a case where x represents 2; R¹ and R⁴ represent an end group having one to four polar groups and 1 to 50 carbon atoms; R¹ and R⁴ may be the same as or different from each other; and at least one of R¹ or R⁴ represents an end group represented by Formula (2)), 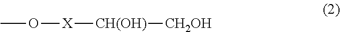
(in Formula (2), X represents a divalent organic group having 2 to 30 carbon atoms, which may have at least one of one or two polar groups or one to three ether oxygen atoms; and X has at least one carbon atom which is not bonded to either the polar groups or the ether oxygen atoms).

2. The fluorine-containing ether compound according to claim 1,
wherein the end group represented by Formula (2) is a group represented by any of Formulae (2-1) to (2-7), 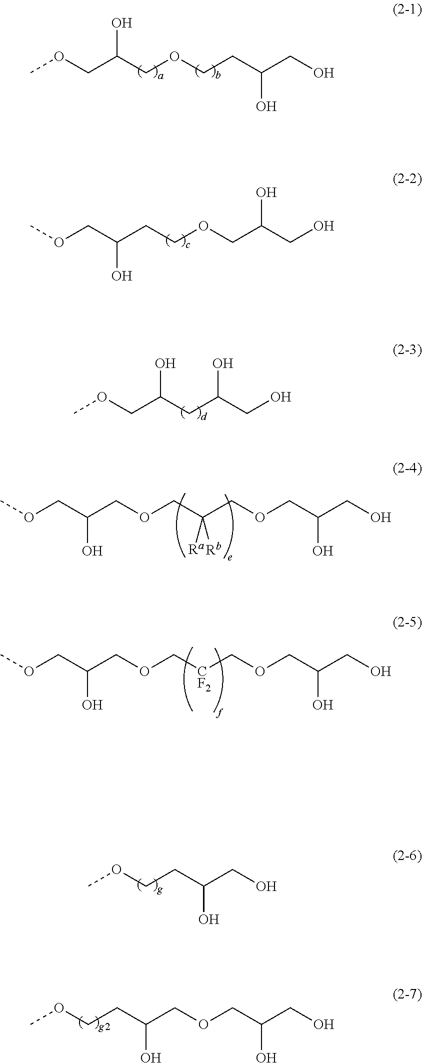
(in Formula (2-1), a represents an integer of 1 to 8; and b represents an integer of 1 to 7),
(in Formula (2-2), c represents an integer of 1 to 7),
(in Formula (2-3), d represents an integer of 1 to 6),
(in Formula (2-4), e represents an integer of 1 to 6; and e pieces of R^{a}'s and R^{b}'s each independently represent a hydrogen atom or a methyl group),
(in Formula (2-5), f represents an integer of 1 to 6),
(in Formula (2-6), g represents an integer of 1 to 6),
(in Formula (2-7), g2 represents an integer of 1 to 6).

3. The fluorine-containing ether compound according to claim 1,
wherein R¹ and R⁴ in Formula (1) each independently represent the end group represented by Formula (2).

4. The fluorine-containing ether compound according to claim 1,
wherein R¹ and R⁴ in Formula (1) are the same as each other.

5. The fluorine-containing ether compound according to claim 1,
wherein one of R¹ or R⁴ in Formula (1) represents the end group represented by Formula (2) and the other represents an end group represented by Formula (3) and not corresponding to Formula (2), 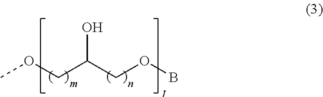
(in Formula (3), l represents an integer of 1 to 3; l pieces of m's each independently represent an integer of 1 to 6; l pieces of n's each independently represent an integer of 1 to 6; in one repeating unit, at least one of m or n represents 1; and B represents an alkyl group which may have only one polar group, an organic group having one or more carbon-carbon unsaturated bonds, or a hydrogen atom).

6. The fluorine-containing ether compound according to claim 1,
wherein one of R¹ or R⁴ in Formula (1) represents the end group represented by Formula (2) and the other represents an end group represented by Formula (3-1) or (3-2), 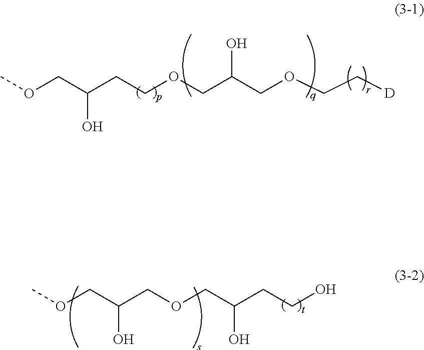
(in Formula (3-1), p represents an integer of 0 to 3; q represents an integer of 0 to 2; r represents an integer of 0 to 5; a total value of p and r is in a range of 1 to 5; and D represents a polar group, a vinyl group, an ethynyl group, or an aryl group which may have a substituent),
(in Formula (3-2), s represents an integer of 0 to 2; and t represents an integer of 1 to 5).

7. The fluorine-containing ether compound according to claim 1,
wherein the x pieces of R³'s in Formula (1) each independently represent a divalent linking group having 3 to 50 carbon atoms, which has one to three hydroxy groups and has an oxygen atom at both ends bonded to adjacent methylene groups.

8. The fluorine-containing ether compound according to claim 1,
wherein the x pieces of R³'s in Formula (1) each independently represent any one selected from the group consisting of linking groups represented by Formulae (4-1) to (4-6), 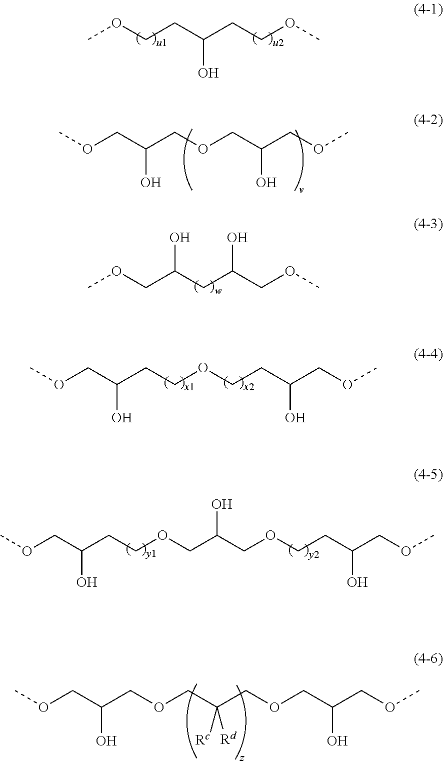
(in Formula (4-1), u1 represents an integer of 0 to 6, u2 represents an integer of 0 to 6, where at least one of u1 or u2 represents 0; and an oxygen atom at a left end of Formula (4-1) is bonded to a methylene group on an R¹ side in Formula (1), and an oxygen atom at a right end is bonded to a methylene group on an R⁴ side in Formula (1)),
(in Formula (4-2), v represents an integer of 1 or 2; and an oxygen atom at a left end of Formula (4-2) is bonded to a methylene group on an R¹ side in Formula (1), and an oxygen atom at a right end is bonded to a methylene group on an R⁴ side in Formula (1)),
(in Formula (4-3), w represents an integer of 0 to 6; and an oxygen atom at a left end of Formula (4-3) is bonded to a methylene group on an R¹ side in Formula (1), and an oxygen atom at a right end is bonded to a methylene group on an R⁴ side in Formula (1)),
(in Formula (4-4), x1 represents an integer of 0 to 5, x2 represents an integer of 0 to 5, where at least one of x1 or x2 represents an integer of 1 to 5; and an oxygen atom at a left end of Formula (4-4) is bonded to a methylene group on an R¹ side in Formula (1), and an oxygen atom at a right end is bonded to a methylene group on an R⁴ side in Formula (1)),
(in Formula (4-5), y1 represents an integer of 1 to 5, y2 represents an integer of 1 to 5; and an oxygen atom at a left end of Formula (4-5) is bonded to a methylene group on an R¹ side in Formula (1), and an oxygen atom at a right end is bonded to a methylene group on a R⁴ side in Formula (1)),
(in Formula (4-6), z represents an integer of 1 to 6; z pieces of R^{c}'s and R^{d}'s each independently represent a hydrogen atom, a fluorine atom, or a methyl group; and an oxygen atom at a left end of Formula (4-6) is bonded to a methylene group on an R¹ side in Formula (1), and an oxygen atom at a right end is bonded to a methylene group on a R⁴ side in Formula (1)).

9. The fluorine-containing ether compound according to claim 1,
wherein a total number of the polar groups of R¹, the polar groups of R⁴, and the polar groups of the x pieces of R³'s in Formula (1) is in a range of 6 to 12.

10. The fluorine-containing ether compound according to claim 1,
wherein the (x+1) pieces of R²'s in Formula (1) each independently represent a perfluoropolyether chain represented by Formula (5), 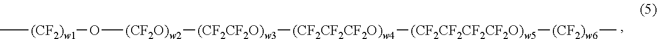
(in Formula (5), each w2, w3, w4, and w5 represents an average degree of polymerization, and each independently represent 0 to 20, where all of w2, w3, w4, and w5 do not represent 0 at the same time; each w1 and w6 represents an average value representing the number of CF₂'s, and each independently represent 1 to 3; and an arrangement order of (CF₂O), (CF₂CF₂O), (CF₂CF₂CF₂O), and (CF₂CF₂CF₂CF₂O), which are repeating units in Formula (5), is not particularly limited).

11. The fluorine-containing ether compound according to claim 1,
wherein the (x+1) pieces of R² in Formula (1) each independently represent any one selected from the group consisting of perfluoropolyether chains represented by Formulae (5-1) to (5-4), 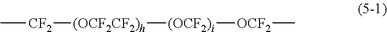
(in Formula (5-1), each h and i represents an average degree of polymerization, where h represents 1 to 20 and i represents 0 to 20), 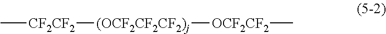
(in Formula (5-2), j represents an average degree of polymerization, and represents 1 to 15), 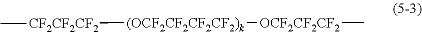
(in Formula (5-3), k represents an average degree of polymerization, and represents 1 to 10), 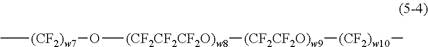
(in Formula (5-4), each w8 and w9 represents an average degree of polymerization, and each independently represents 1 to 20; and each w7 and w10 represents an average value representing the number of CF₂'s, and each independently represent 1 to 2).

12. The fluorine-containing ether compound according to claim 1,
wherein the fluorine-containing ether compound has a number-average molecular weight of 500 to 10000.

13. A lubricant for a magnetic recording medium, comprising:
the fluorine-containing ether compound according to claim 1.

14. A magnetic recording medium, which is provided with at least a magnetic layer, a protective layer, and a lubricating layer in this order on a substrate,
wherein the lubricating layer contains the fluorine-containing ether compound according to claim 1.

15. The magnetic recording medium according to claim 14,
wherein the lubricating layer has an average film thickness of 0.5 nm to 2.0 nm.
